(19) United States
(12) Patent Application Publication
(10) Pub. No.: US 2024/0226277 A1
(43) Pub. Date: Jul. 11, 2024

(54) INFLUENZA-CORONAVIRUS COMBINATION VACCINES

(71) Applicant: ModernaTX, Inc., Cambridge, MA (US)

(72) Inventors: Raffael Nachbagauer, Cambridge, MA (US); Carole Henry, Cambridge, MA (US); Guillaume Stewart-Jones, Cambridge, MA (US); Elsabeth Narayanan, Cambridge, MA (US); Humilton Bennett, Cambridge, MA (US); Andrea Carfi, Cambridge, MA (US)

(73) Assignee: ModernaTX. Inc., Cambridge, MA (US)

(21) Appl. No.: 18/555,130

(22) PCT Filed: Apr. 13, 2022
(86) PCT No.: PCT/US2022/024669

§ 371 Date: Oct. 12, 2023

Related U.S. Application Data
(60) Provisional application No. 63/242,346, filed on Sep. 9, 2021.
(60) Provisional application No. 63/175,007, filed on Apr. 14, 2021.

(51) Int. Cl. A61K 39/215; A61K 9/51; A61K 39/00; A61K 39/145; A61K 47/16; A61P 37/04

(52) U.S. Cl. CPC A61K 39/215; A61K 9/5123; A61K 39/145; A61K 47/16; A61P 37/04; A61K 2039/53; A61K 2039/70

(57) ABSTRACT

The disclosure provides combination miRNA vaccines for respiratory viruses, such as influenza and coronaviruses (e.g., SARS-CoV-2) as well as methods of using the vaccines.

### RELATED APPLICATIONS

This application claims priority under 35 U.S.C. § 119(e) to U.S. Provisional Patent Application No. 63/175,007, filed Apr. 14, 2021, and U.S. Provisional Patent Application No. 63/242,346, filed Sep. 9, 2021, which are hereby incorporated by reference in their entireties.

### REFERENCE TO SEQUENCE LISTING SUBMITTED AS TEXT FILE VIA EFS-WEB

The instant application contains a Sequence Listing which has been submitted in ASCII format via EFS-Web and is hereby incorporated by reference in its entirety. Said ASCII copy, created on Apr. 13, 2022, is named M137870180W000-JXV-SEQ and is 202,400 bytes in size.

### BACKGROUND

Respiratory disease is a medical term that encompasses pathological conditions affecting the organs and tissues that make gas exchange possible in higher organisms, and includes conditions of the upper respiratory tract, trachea, bronchi, bronchioles, alveoli, pleura and pleural cavity, and the nerves and muscles of breathing. Respiratory diseases range from mild and self-limiting, such as the common cold, to life-threatening entities like bacterial pneumonia, pulmonary embolism, acute asthma, and lung cancer. Respiratory disease is a common and significant cause of illness and death around the world. In the US, approximately 1 billion “common colds” occur each year. Respiratory conditions are among the most frequent reasons for hospital stays among children.

Seasonal influenza is an acute respiratory infection caused by influenza viruses-influenza A and influenza B viruses, which are members of the Orthomyxoviridae family—that circulate in all parts of the world. Seasonal influenza is characterized by a sudden onset of fever, cough (usually dry), headache, muscle and joint pain, severe malaise (feeling unwell), sore throat and a runny nose. In industrialized countries most deaths associated with influenza occur among people age 65 or older. Epidemics can result in high levels of worker/school absenteeism and productivity losses. Clinics and hospitals can be overwhelmed during peak illness periods. The effects of seasonal influenza epidemics in developing countries are not fully known, but research estimates that 99% of deaths in children under 5 years of age with influenza related lower respiratory tract infections are found in developing countries.

Human coronaviruses are highly contagious enveloped, positive sense single-stranded RNA viruses of the Coronaviridae family. Two sub-families of Coronaviridae are known to cause human disease. The most important being the 0-coronaviruses (betacoronaviruses). The 0-coronaviruses are common etiological agents of mild to moderate upper respiratory tract infections. Outbreaks of novel coronavirus infections such as the infections caused by a coronavirus initially identified from the Chinese city of Wuhan in December 2019; however, have been associated with a high mortality rate death toll. This recently identified coronavirus, referred to as Severe Acute Respiratory Syndrome Coronavirus 2 (SARS-CoV-2) (formerly referred to as a “2019 novel coronavirus,” or a “2019-nCoV”) has rapidly infected millions of people. The pandemic disease that the SARS-CoV-2 virus causes has been named by World Health Organization (WHO) as COVID-19 (Coronavirus Disease 2019). The first genome sequence of a SARS-CoV-2 isolate (Wuhan-Hu-1; USA-WA1/2020 isolate) was released by investigators from the Chinese CDC in Beijing on Jan. 10, 2020 at Virological, a UK-based discussion forum for analysis and interpretation of virus molecular evolution and epidemiology. The sequence was then deposited in GenBank on Jan. 12, 2020, having Genbank Accession number MN908947.1. Subsequently, a number of SARS-CoV-2 strain variants have been identified, some of which are more infectious than the SARS-CoV-2 isolate.

The continuing health problems associated with respiratory viruses, such as influenza, and coronaviruses, are of concern internationally, reinforcing the importance of developing effective and safe vaccine candidates against these viruses.

### SUMMARY

The disclosure, in some aspects, provides a combination vaccine, comprising a first messenger ribonucleic acid (mRNA) polynucleotide comprising an open reading frame (ORF) encoding a first respiratory virus antigenic polypeptide, wherein the first respiratory virus antigenic polypeptide is an influenza virus antigen; and a second mRNA polynucleotide comprising an ORF encoding a second respiratory virus antigenic polypeptide from a coronavirus; and a lipid nanoparticle.

In another aspect, the disclosure provides a combination vaccine, comprising a first messenger ribonucleic acid (mRNA) polynucleotide comprising an open reading frame (ORF) encoding a first respiratory virus antigenic polypeptide, wherein the first respiratory virus antigenic polypeptide is an influenza virus antigen; a second mRNA polynucleotide comprising an ORF encoding a second respiratory virus antigenic polypeptide from a second influenza virus; a third mRNA polynucleotide comprising an ORF encoding a third respiratory virus antigenic polypeptide from a third influenza virus; a fourth mRNA polynucleotide comprising an ORF encoding a fourth respiratory virus antigenic polypeptide from a fourth influenza virus; a fifth mRNA polynucleotide comprising an ORF encoding a fifth respiratory virus antigenic polypeptide from a first coronavirus; a sixth mRNA polynucleotide comprising an ORF encoding a sixth respiratory virus antigenic polypeptide from a second coronavirus; and a lipid nanoparticle.

In some embodiments, the first, second, third and fourth viruses are selected from influenza A viruses and influenza B viruses. In some embodiments, the second virus is a betacoronavirus. In some embodiments, the coronavirus (e.g., first coronavirus, second coronavirus, or both the first and the second coronavirus) is selected from the group consisting of MERS-CoV, SARS-CoV, SARS-CoV-2, HCoV-OC43, HCoV-229E, HCoV-NL63, HCoV-NL, HCoV-NH and HCoV-HKU1.

In some embodiments, the first respiratory virus antigenic polypeptide is from an influenza virus B. In some embodiments, the first respiratory virus antigenic polypeptide is from an influenza virus A. In some embodiments, the first respiratory virus antigenic polypeptide is hemagglutinin antigen (HA) or a neuraminidase antigen (NA).

In some embodiments, the second respiratory virus antigenic polypeptide is from a SARS-CoV. In some embodiments, the second respiratory virus antigenic polypeptide is from SARS-CoV-2. In some embodiments, the second respiratory virus antigenic polypeptide is from a non-SARS human coronavirus (HCoV).

In some embodiments, the vaccine comprises at least 2 mRNA polynucleotides comprising an ORF encoding an influenza virus antigen. In some embodiments, the vaccine comprises 2-4 mRNA polynucleotides comprising an ORF encoding an influenza virus antigen.

In some embodiments, the vaccine comprises at least 2 mRNA polynucleotides comprising an ORF encoding a respiratory virus antigenic polypeptide from a coronavirus.

In some embodiments, the vaccine comprises less than 15 mRNA polynucleotides. In some embodiments, the vaccine comprises 3-10 mRNA polynucleotides. In some embodiments, the vaccine comprises 4-10 mRNA polynucleotides. In some embodiments, the vaccine comprises 5-10 mRNA polynucleotides. In some embodiments, the vaccine comprises 8-9 mRNA polynucleotides.

In some embodiments, the vaccine comprises at least three mRNA polynucleotides encoding influenza virus antigenic polypeptides. In some embodiments, the vaccine comprises at least eight mRNA polynucleotides encoding influenza virus antigenic polypeptides. In some embodiments, the vaccine comprises at least two mRNA polynucleotides encoding coronavirus antigenic polypeptides.

In some embodiments, the first and second mRNA polynucleotides are present in the combination vaccine in a ratio of 1:1. In some embodiments, the combination vaccine comprises a ratio of mRNA polynucleotides encoding respiratory virus antigenic polypeptides of 4:1 from the influenza virus to the coronavirus. In some embodiments, the combination vaccine comprises a ratio of mRNA polynucleotides encoding respiratory virus antigenic polypeptides of 3:1 from the influenza virus to the coronavirus. In some embodiments, the combination vaccine comprises a ratio of mRNA polynucleotides encoding respiratory virus antigenic polypeptides of 2:1 from the influenza virus to the coronavirus. In some embodiments, the combination vaccine comprises a ratio of mRNA polynucleotides encoding respiratory virus antigenic polypeptides of 5:1 from the influenza virus to the coronavirus. In some embodiments, the combination vaccine comprises a ratio of mRNA polynucleotides encoding respiratory virus antigenic polypeptides of 4:2 from the influenza virus to the coronavirus. In some embodiments, the combination vaccine comprises a ratio of mRNA polynucleotides encoding respiratory virus antigenic polypeptides of 1:2 from the influenza virus to the coronavirus. In some embodiments, the combination vaccine comprises a ratio of mRNA polynucleotides encoding respiratory virus antigenic polypeptides of 8:2 from the first virus to the second virus. In some embodiments, the combination vaccine comprises a ratio of mRNA polynucleotides encoding respiratory virus antigenic polypeptides of 8:1 from the first virus to the second virus. In some embodiments, the respiratory virus antigenic polypeptides of the first virus comprise HAs and NAs, in a ratio of 4:4.

In some embodiments, each of the mRNA polynucleotides in the combination vaccine is complementary with and does not interfere with each other mRNA polynucleotide in the combination vaccine.

In some embodiments, at least one of the respiratory virus antigenic polypeptides is derived from a naturally occurring antigen. In some embodiments, at least one of the respiratory virus antigenic polypeptides is a stabilized version of a naturally occurring antigen. In some embodiments, at least one of the respiratory virus antigenic polypeptides is a non-naturally occurring antigen.

In some embodiments, the vaccine further comprises an mRNA polynucleotide encoding a structurally altered variant respiratory virus antigenic polypeptide, wherein the structurally altered variant is a structurally altered variant of any one of the first or second respiratory virus antigenic polypeptides.

In some embodiments, at least one of the first and second mRNA polynucleotides is polycistronic. In some embodiments, each of the first and second mRNA polynucleotides is polycistronic.

Another aspect of the disclosure provides a multivalent RNA composition comprising a first messenger ribonucleic acid (mRNA) polynucleotide comprising an open reading frame (ORF) encoding a first respiratory virus antigenic polypeptide, from a first virus; and a second mRNA polynucleotide comprising an ORF encoding a second respiratory virus antigenic polypeptide from a coronavirus; wherein the multivalent RNA composition comprises greater than 40% polyA-tailed RNAs and/or the first and/or second mRNA polynucleotides is different in length from one another by at least 100 nucleotides.

In some embodiments, the composition is produced by a method comprising (a) combining a linearized first DNA molecule encoding the first mRNA polynucleotide and a linearized second DNA molecule encoding the second mRNA polynucleotide into a single reaction vessel, wherein the first DNA molecule and the second DNA molecule are obtained from different sources; and (b) simultaneously in vitro transcribing the linearized first DNA molecule and the linearized second DNA molecule to obtain a multivalent RNA composition.

In some embodiments, the different sources are a first and second bacterial cell culture and wherein the first and second bacterial cell culture are not co-cultured. In some embodiments, the amounts of the first and second DNA molecules present in the reaction mixture prior to the start of the IVT have been normalized.

In some embodiments, wherein the coronavirus is selected from the group consisting of MERS-CoV, SARS-CoV, SARS-CoV-2, HCoV-OC43, HCoV-229E, HCoV-NL63, HCoV-NL, HCoV-NH and HCoV-HKU1.

Another aspect of the disclosure provides a multivalent RNA composition comprising 2-15 mRNA polynucleotides, each comprising a distinct open reading frame (ORF) encoding a respiratory virus antigenic polypeptide, wherein at least one respiratory virus antigenic polypeptide is an influenza virus and at least one respiratory virus antigenic polypeptide is a coronavirus, and wherein each mRNA polynucleotide comprises one or more non-coding sequence in an untranslated region (UTR), optionally a 5′ UTR or 3′ UTR.

In some embodiments, the non-coding sequence is positioned in a 3′ UTR of an mRNA, upstream of the polyA tail of the mRNA.

In some embodiments, the non-coding sequence is positioned in a 3′ UTR of an mRNA, downstream of the polyA tail of the mRNA.

In some embodiments, the non-coding sequence is positioned in a 3′ UTR of an mRNA between the last codon of the ORF of the mRNA and the first “A” of the polyA tail of the mRNA. In some embodiments, the non-coding sequence comprises between 1 and 10 nucleotides. In some embodiments, the non-coding sequence comprises one or more RNAse cleavage sites. In some embodiments, the RNAse cleavage site is an RNase H cleavage site.

In some embodiments, the coronavirus antigen is selected from the group consisting of MERS-CoV, SARS-CoV, SARS-CoV-2, HCoV-OC43, HCoV-229E, HCoV-NL63, HCoV-NL, HCoV-NH and HCoV-HKU1.

The disclosure, in some aspects, provides a multivalent RNA composition, comprising a first messenger ribonucleic acid (mRNA) polynucleotide comprising an open reading frame (ORF) encoding a first respiratory virus antigenic polypeptide, from an influenza virus; a second mRNA polynucleotide comprising an ORF encoding a second respiratory virus antigenic polypeptide from a coronavirus; and wherein at least one of the respiratory virus antigenic polypeptides is derived from a naturally occurring antigen or a stabilized version of a naturally occurring antigen and further comprising an mRNA polynucleotide encoding a structurally altered variant respiratory virus antigenic polypeptide, wherein the structurally altered variant is a structurally altered variant of any one of the first or second respiratory virus antigenic polypeptides.

In some embodiments, the coronavirus is selected from the group consisting of MERS-CoV, SARS-CoV, SARS-CoV-2, HCoV-OC43, HCoV-229E, HCoV-NL63, HCoV-NL, HCoV—NH and HCoV-HKU1.

In some embodiments, the structurally altered variant is a structurally altered variant of the first respiratory virus antigenic polypeptide.

In some embodiments, the structurally altered variant is a structurally altered variant of the second respiratory virus antigenic polypeptide.

Another aspect of the disclosure provides a multivalent RNA composition, comprising 5 to 15 messenger ribonucleic acid (mRNA) polynucleotides, each comprising an open reading frame (ORF) encoding a distinct respiratory virus antigenic polypeptide, wherein the respiratory virus antigenic polypeptides are derived from two different viral families, wherein the two viral families comprise influenza viruses and coronaviruses; and a lipid nanoparticle.

In some embodiments, the composition has 3-6 mRNA polynucleotides comprising an ORF encoding an influenza antigen. In some embodiments, the composition has 1-5 mRNA polynucleotides comprising an ORF encoding a coronavirus antigen.

The disclosure, in some aspects, provides a multivalent RNA composition, comprising a set of at least 6 messenger ribonucleic acid (mRNA) polynucleotides, each comprising an open reading frame (ORF) encoding a respiratory virus antigenic polypeptide from a first or second virus; wherein the first virus is an influenza virus, wherein the second virus is a coronavirus, and wherein the composition comprises a ratio of mRNA polynucleotides encoding respiratory virus antigenic polypeptides of 4:1, 4:2, or 4:3 from the first virus to the second virus.

In some embodiments, the first and second mRNA polynucleotides are present in the combination vaccine in a ratio of 1:1. In some embodiments, the multivalent RNA composition comprises a ratio of mRNA polynucleotides encoding respiratory virus antigenic polypeptides of 4:1 from the first virus to the second virus. In some embodiments, the multivalent RNA composition comprises a ratio of mRNA polynucleotides encoding respiratory virus antigenic polypeptides of 3:1 from the first virus to the second virus. In some embodiments, the multivalent RNA composition comprises a ratio of mRNA polynucleotides encoding respiratory virus antigenic polypeptides of 2:1 from the first virus to the second virus. In some embodiments, the multivalent RNA composition comprises a ratio of mRNA polynucleotides encoding respiratory virus antigenic polypeptides of 5:1 from the first virus to the second virus. In some embodiments, the multivalent RNA composition comprises a ratio of mRNA polynucleotides encoding respiratory virus antigenic polypeptides of 4:2 from the first virus to the second virus.

In some embodiments, the multivalent RNA composition comprises a ratio of mRNA polynucleotides encoding respiratory virus antigenic polypeptides of 1:2 from the first virus to the second virus. In some embodiments, the multivalent RNA composition comprises a ratio of mRNA polynucleotides encoding respiratory virus antigenic polypeptides of 8:1 or 8:2 from the first virus to the second virus.

In some embodiments, the antigenic polypeptides include a Fusion (F) protein, a spike (S) protein, and a hemagglutinin antigen (HA). In some embodiments, the multivalent RNA compositions described herein further comprise a neuraminidase (NA) antigen.

In some embodiments, the multivalent RNA compositions described herein further comprise at least one lipid nanoparticle (LNP). In some embodiments, the LNP comprises a molar ratio of 20-60% ionizable amino lipid, 5-25% non-cationic lipid, 25-55% sterol, and 0.5-15% PEG-modified lipid. In some embodiments, the ionizable amino lipid comprises the structure of Compound 1:

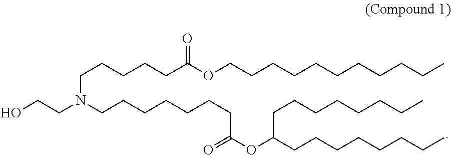 [SMILES: CCCCCCCCCCCOC(=O)CCCCCN(CCO)CCCCCCCC(=O)OC(CCCCCCCC)CCCCCCCC]

In some embodiments, the respiratory virus antigenic polypeptide comprises a cell surface glycoprotein.

The disclosure, in some aspects, provides a method for vaccinating a subject, comprising administering to the subject a combination vaccine, wherein the combination vaccine comprises a first messenger ribonucleic acid (mRNA) polynucleotide comprising an open reading frame (ORF) encoding a first respiratory virus antigenic polypeptide from an influenza virus; and a second mRNA polynucleotide comprising an ORF encoding a second respiratory virus antigenic polypeptide from a coronavirus.

In some embodiments, the subject is 65 years of age or older. In some embodiments, the subject is under 18 years of age.

In some embodiments, the method prevents a respiratory infection in the subject. In some embodiments, the method reduces the severity of a respiratory infection in the subject.

In some embodiments, the subject is seronegative for at least one of the antigenic polypeptides. In some embodiments, the subject is seronegative for all of the antigenic polypeptides. In some embodiments, the subject is seropositive for at least one of the antigenic polypeptides. In some embodiments, the subject is seropositive for all of the antigenic polypeptides.

In some embodiments, any of the methods disclosed herein further comprise administering a booster vaccine. In some embodiments, the booster vaccine is administered between 3 weeks and 1 year after the combination vaccine.

In some embodiments, the booster vaccine comprises at least one mRNA polynucleotide comprising an ORF encoding the first or second respiratory virus antigenic polypeptides. In some embodiments, the booster vaccine comprises at least one mRNA polynucleotide comprising an ORF encoding the first and second respiratory virus antigenic polypeptides. In some embodiments, the booster vaccine comprises at least one mRNA polynucleotide comprising an ORF encoding a structurally altered variant of the first or second respiratory virus antigenic polypeptides.

In some embodiments, the combination vaccine is a seasonal booster vaccine.

In some embodiments, the combination vaccine is any of the vaccines disclosed herein.

The disclosure, in some embodiments, provides a method of preventing or reducing the severity of a respiratory infection by administering the combination/multivalent vaccine described herein to a subject in an effective amount to prevent infection or reduce the severity of a respiratory infection in the subject based on a single dose or single dose with a booster.

In some embodiments, the combination vaccine is administered to the subject in a dose of 50 μg. In some embodiments, the combination vaccine is administered to the subject in a dose of 25 μg. In some embodiments, the combination vaccine is administered to the subject in a dose of 100 μg.

In some embodiments, each RNA polynucleotide of the vaccine is formulated in a separate LNP. In some embodiments, the RNA polynucleotides of the vaccine are co-formulated in an LNP.

In some embodiments, any of the compositions or vaccines described herein (e.g., for use in any of the methods described herein) comprise mRNA polynucleotides encoding four HA antigens. In some embodiments, four HA antigens are present in a 1:1:1:1 ratio.

In some embodiments, any of the compositions or vaccines described herein further comprising mRNA polynucleotides encoding four NA antigens. In some embodiments, the four NA antigens are present in a 1:1:1:1 ratio.

In some embodiments, the ratio of HA antigens to NA antigens is 1:1. In some embodiments, the ratio of HA antigens to NA antigens is 3:1.

In some embodiments, any of the compositions described herein (e.g., for the use in any of the methods described herein), the coronavirus is a betacoronavirus.

### BRIEF DESCRIPTION OF THE DRAWINGS

FIG. 1 is a series of graphs showing the hemagglutinin (HA)-reactive IgG antibody titers to each of the four HA antigens 21 days after one dose of the formulations indicated.

FIG. 2 is a series of graphs showing the NA-reactive IgG antibody titers to each of the four NA antigens 21 days after one dose of the formulations indicated.

FIG. 3 is a graph showing the SARS-CoV-2 S2P-specific IgG antibody titers 21 days after one dose of the formulations indicated.

FIG. 4 is a series of graphs showing the normalized hemagglutinin (HA)-reactive IgG antibody titers to each of the four HA antigens 21 days after one dose of the formulations indicated.

FIG. 5 is a graph showing the normalized SARS-CoV-2 S2P-specific IgG antibody titers 21 days after one dose of the formulations indicated.

FIG. 6 is a graph showing the SARS-CoV-2 S2P-specific IgG antibody titers 21 days after one dose of the formulations indicated.

FIG. 7 is a graph showing the SARS-CoV-2 B.1.351 variant-specific IgG antibody titers 21 days after one dose of the formulations indicated.

FIG. 8 is a graph showing the hemagglutinin (HA)-reactive IgG antibody titers to the H1 HA Wisconsin antigen (SEQ ID NO: 22) 21 days after one dose of the formulations indicated.

FIG. 9 is a graph showing the hemagglutinin (HA)-reactive IgG antibody titers to the H3 HA Hong Kong antigen (SEQ ID NO: 19) 21 days after one dose/36 days after two doses of the formulations indicated.

FIG. 10 is a graph showing the hemagglutinin (HA)-reactive IgG antibody titers to the B HA Phuket antigen (SEQ ID NO: 21) 21 days after one dose/36 days after two doses of the formulations indicated.

FIG. 11 is a graph showing the hemagglutinin (HA)-reactive IgG antibody titers to the B HA Washington antigen (SEQ ID NO: 20) 21 days after one dose of the formulations indicated.

### DETAILED DESCRIPTION

Respiratory viruses are the most common agents of disease in humans, having a significant impact on morbidity and mortality worldwide. Certain respiratory agents from several virus families are well-suited to efficient person-to-person transmission, leading to global circulation. Community-based studies have confirmed that these viruses are the most prevalent etiological agents of acute respiratory infections. Effective vaccines and antiviral drugs are not yet available for most of these viruses.

The present disclosure therefore provides, in some embodiments, combination vaccines that comprise RNA (e.g., mRNA) polynucleotides encoding at least two respiratory antigenic polypeptides from at least two different respiratory viruses. In some embodiments the two different viruses are from the Orthomyxoviridae and Coronaviridae (optionally, Orthocoronavirinae) families. In some embodiments, the respiratory antigenic polypeptides are from an Alphainfluenzavirus genus, a Betainfluenzavirus genus or a Betacoronavirus genus.

Combination RNA vaccines have been challenging to make as a result of synthesis, formulation, and delivery limitations. Combinations of two or more RNA polynucleotides encoding respiratory antigens in lipid nanoparticle carriers are disclosed herein. Methods for successfully generating functional combinations of RNA polynucleotides encoding antigens to produce highly effective combination vaccines are disclosed herein. One limitation of combination vaccines relates to interference between antigens such that a complete and robust immune response is not generated against all of the antigens in the vaccine. It has been demonstrated that combination vaccines encoding multiple antigens, i.e., 8-10 antigens can be generated and still produce a complete immune response. In some embodiments, each of the mRNA polynucleotides in the combination vaccine is complementary with and does not interfere with each other mRNA polynucleotide in the combination vaccine. Thus, the antigens produced from administration of the combination vaccine do not interfere with immune response of one another. As presented in the data described in the Examples, administration of combination vaccines comprising mRNA polynucleotides encoding antigens from the Orthomyxoviridae family (e.g., influenza antigens) and the Coronaviridae family (e.g., SARS-CoV-2), quite surprisingly, did not inhibit or reduce the neutralizing antibody titers for each respective antigen relative to administration of mRNA encoding each single antigen separately.

Also provided herein are methods of administering the vaccines, methods of producing the vaccines, compositions comprising the vaccines, and nucleic acids encoding the vaccines. As described herein, the vaccines described herein may be used to induce a balanced immune response, comprising both cellular and humoral immunity, without many of the risks associated with DNA vaccination. Such a vaccine, optionally referred to herein as a multivalent vaccine or combination vaccine, can be administered to seropositive or seronegative subjects. For example, a subject may be naive and not have antibodies that react with at least one of the respiratory virus antigenic polypeptides of the vaccine, or may have preexisting antibodies to at least one of respiratory virus antigens of the vaccine because they have previously had an infection with the respiratory virus or may have previously been administered a dose of a vaccine (e.g., an mRNA vaccine) that induces antibodies against the respiratory virus. In some embodiments, a subject may have preexisting antibodies to all of respiratory virus antigens of the vaccine.

### Antigens and Combination Vaccines

Antigens, as used herein, are proteins capable of inducing an immune response (e.g., causing an immune system to produce antibodies against the antigens). The vaccines of the present disclosure provide a unique advantage over traditional protein-based vaccination approaches in which protein antigens are purified or produced in vitro, e.g., recombinant protein production technologies. The vaccines of the present disclosure feature mRNA encoding the desired antigens, which when introduced into the body, i.e., administered to a mammalian subject (for example a human) in vivo, cause the cells of the body to express the desired antigens. The vaccines of the present disclosure feature mRNA encoding the desired viral surface antigens, e.g., glycoprotein antigens, which when introduced into the body, i.e., administered to a mammalian subject (for example a human) in vivo, cause the cells of the body to express the desired peptides in a native fold and, optionally with human glycosylation patterns. Thus, a combination vaccine encoding the viral surface antigen from a series of pathogenic viruses all presenting the properly folded and, optionally, glycosylated viral antigens in the same manner as if it was generated during an actual infection. Thus, mRNA vaccines thus offer the best vehicle for making vaccines to respiratory viruses one can produce short of using an attenuated virus but without the associated risks. In order to facilitate delivery of the mRNAs of the present disclosure to the cells of the body, the mRNAs are encapsulated in lipid nanoparticles (LNPs). Upon delivery and uptake by cells of the body, the mRNAs are translated in the cytosol and protein or glycoprotein antigens are folded and processed by the host cell machinery. The protein and/or glycoprotein antigens are presented and elicit an adaptive humoral and cellular immune response. Neutralizing antibodies are directed against the expressed viral receptor binding protein and glycoprotein antigens and hence these viral protein antigens are considered the most relevant target antigens for vaccine development. Simply put, neutralizing antibodies are generally directed to the viral surface proteins, e.g., glycoproteins, which are responsible for binding to the cell and when blocked by a specific antibody, the virus is neutralized. Herein, use of the term “antigen” encompasses immunogenic viral surface proteins, e.g., glycoproteins, and immunogenic fragments (an immunogenic fragment that induces (or is capable of inducing) an immune response to a (at least one) respiratory virus), unless otherwise stated. In some embodiments, the antigen is a naturally occurring antigen (e.g., the respiratory virus antigenic polypeptide encodes a naturally occurring antigen). In some embodiments, at least one respiratory virus antigenic polypeptide is a non-naturally occurring antigen or an engineered version of the protein or glycoprotein antigen for use in a combination vaccine. In some embodiments, at least one of the respiratory virus antigenic polypeptides is a stabilized version of a naturally occurring antigen (e.g., a coronavirus Spike protein stabilized by one or more amino acid substitutions, additions, or deletions, e.g., two proline substitutions). In another embodiment, other modifications are engineered into the viral surface protein, e.g., glycoprotein, such as deletion of cytoplasmic tails or mutations to facilitate protein processing or conformational stability.

It should be understood that the term “protein” encompasses glycoproteins, proteins, peptides and fragments thereof and the term “antigen” encompasses antigenic portions of such molecules that provoke an immune response. For the viral vaccines included herein, the term “antigen” includes viral surface proteins, e.g., glycoproteins, fragments of viral proteins (e.g., glycoproteins) and designed and or mutated versions of viral proteins (e.g., glycoproteins) derived from respiratory viruses.

### Orthomyxoviridae Family

The Orthomyxoviridae family is a family of negative-sense RNA viruses and incudes Alphainfluenzavirus, Betainfluenzavirus, Deltainfluenzavirus, Gammainfluenzavirus, Isavirus, Thogotovirus, and Quaranjavirus. The vaccines described herein may comprise viral antigenic polypeptides from Alphainfluenzavirus or Betainfluenzavirus. Both are associated with human influenzas.

All influenza viruses are negative-strand RNA viruses with a segmented genome. Influenza type A and B viruses have 8 genes that code for 10 Proteins, including the surface proteins hemagglutinin (HA) and neuraminidase (NA), In the case of influenza type A, viruses, further subdivision can be made into different subtypes according to differences in these, two surface proteins. To date, 16 HA subtypes and 9 NA subtypes have been identified. However, during the 20th century, the only influenza A subtypes that circulated extensively in humans were A(H1N1); A(H1N2): A(H2N2); and A(H3N2). All known subtypes Of influenza type A viruses have been isolated from birds and can affect a range of mammal species. As with humans, the number of influenza A subtypes that have been isolated from other mammalian species is limited. Almost all influenza A pandemics have been caused by descendants of the 1918 virus, including “drifted” H1N1 viruses and reassorted H2N2 and H3N2 viruses. Influenza A comprises HA and NA proteins on the surface of its viral envelope, HA allows the virus's recognizing and binding to target cells, and also to infect the cell with viral RNA. NA is critical for the subsequent release of the daughter virus particles created within the infected cell so they can spread to other cells.

Influenza type B viruses almost exclusively infect humans. Influenza B viruses are not classified into subtypes but can be broken down into lineages. Currently circulating influenza type B viruses belong to either B/Yamagata (B/Yamagata/16/88-like) or B/Victoria (B/Victoria/2/87-like) lineage. Influenza virus B mutates at a rate 2 to 3 times slower than type A; however, it significantly impacts children and young adults annually. The influenza B virus capsid is enveloped while, its virion consists of an envelope, a matrix protein, a nucleoprotein complex, a nucleocapsid, and a polymerase complex. It can be spherical or filamentous. Its 500 or so surface, projections are made of HA and NA. The influenza B virus genome is 14,548 nucleotides long and consists of eight segments of linear negative-sense, single-stranded RNA. The multipartite genome is encapsidated, each segment in a separate nucleocapsid, and the nucleocapsid are surrounded by one envelope.

The mRNA vaccines of the instant invention comprise mRNAs encoding HA, and optionally, NA antigens of the influenza viruses circulating at the time of design of the vaccines. Exemplary vaccines of the invention comprise mRNAs encoding HA antigens, and optionally 20 NA antigens of the circulating H1N1 viruses and H3N2 viruses. The vaccines of the invention can comprise mRNAs encoding the HA antigens of each circulating influenza A subtype or of each predominant influenza A subtype in combination with mRNAs encoding the HA antigens of each circulating influenza B lineage (or of each predominant influenza lineage). In exemplary embodiments, the vaccines also comprise mRNAs encoding the NA antigens corresponding to the selected HA antigens. Predominant viruses, or those predominant in circulation, are those detected in the human population at an endemic frequency or at a frequency above a certain threshold understood by the skilled artisan is requisite to evidence that those strain(s) are in circulation within a population, e.g., within populations representative of the Northern or Southern hemisphere.

The mRNA vaccines of the invention are amenable to inclusion of multiple mRNAs and, as such, can include mRNAs encoding, for example, the HA antigens, and optionally also the corresponding NA antigens, of the most prevalent A/H1N1 strain, A/H3N2 strain, B/Victoria lineage and B/Yamagata lineage, but can further include mRNAs encoding the HA antigens, and optionally also the corresponding NA antigens, of a second prevalent A/H1N1 strain, A/H3N2 strain, B/Victoria lineage and/or B/Yamagata lineage. In exemplary embodiments, an mRNA vaccine of the invention includes mRNA encoding the HA antigen of an influenza A virus strain of the A(H1N1) subtype, mRNA encoding the HA antigen of an influenza A virus strain of the A(H3N2) subtype, mRNA encoding the HA antigen of an influenza b virus strain of the B/Victoria lineage and mRNA encoding the HA antigen of an influenza B virus strain of the B/Yamagata lineage.

In some embodiments, the antigen is an influenza antigen. The influenza antigen is hemagglutinin (HA) or neuraminidase (NA). In some embodiments, the influenza antigen is be a fragment of, a derivative of, or a modified HA or NA. For example, in some embodiments, the NA is a wild-type NA (e.g., is enzymatically active). In some embodiments, the NA is a modified NA, such as an enzymatically inactive NA. As used herein, “enzymatically inactive NA” refers to a NA that has been mutated such that it possesses no or minimal catalytic activity (see, e.g., Richard et al., J Clin Virol., 2008, 41(1): 20-24; Yen et al., J Virol., 2006, 80(17): 8787-8795). For example, in some embodiments, the enzymatically inactive NA possesses less than 30%, 25%, 20%, 15%, 14%, 13%, 12%, 11%, 10%, 9%, 8%, 7%, 6%, 5%, 4%, 3%, 2%, 1%, or 0% of the catalytic activity of the wild-type NA (e.g., in an enzymatic activity assay, as is known in the art). In some embodiments, at least one of Arg118, Asp151, Arg152, Arg224, Glu276, Arg292, Arg371 and Tyr406 is mutated. In some embodiments, 1, 2, 3, 4, 5, 6, 7, or all 8 amino acids are mutated. In some embodiments, the mutation is R118K, D151G, is E227D.

In some embodiments, the mRNA vaccines of the present disclosure may comprise a combination of mRNAs encoding HA, optionally in combination with mRNAs encoding NA antigens, or fragments, derivatives, or modified versions thereof. In some embodiments, the mRNA vaccine may comprise a combination of mRNAs encoding HA and mRNAs encoding NA antigens, or fragments, derivatives, or modified versions thereof. In some embodiments, the vaccine comprises mRNAs encoding 1, 2, 3, 4, 5, 6, 7, 8, 9, or 10 HA antigens and/or mRNAs encoding 1, 2, 3, 4, 5, 6, 7, 8, 9, or 10 NA antigens, or any combination thereof (e.g., 4 HA antigens, or 4 HA antigens and 4 NA antigens). In some embodiments, the vaccine comprises mRNA encoding one HA antigen. In some embodiments, the vaccine comprises mRNAs encoding two HA antigens. In some embodiments, the vaccine comprises mRNAs encoding three HA antigens. In some embodiments, the vaccine comprises mRNAs encoding four HA antigens. In some embodiments, the vaccine comprises mRNAs encoding five HA antigens. In some embodiments, the vaccine comprises mRNAs encoding six HA antigens. In some embodiments, the vaccine comprises mRNA encoding one HA antigen and mRNA encoding one NA antigen. In some embodiments, the vaccine comprises mRNAs encoding two HA antigens and mRNAs encoding two NA antigens. In some embodiments, the vaccine comprises mRNAs encoding three HA antigens and mRNAs encoding three NA antigens. In some embodiments, the vaccine comprises mRNAs encoding four HA antigens and mRNAs encoding four NA antigens. In some embodiments, the vaccine comprises mRNAs encoding five HA antigens and mRNAs encoding five NA antigens. In some embodiments, the vaccine comprises mRNAs encoding six HA antigens and mRNAs encoding six NA antigens.

By virtue of the multiple mRNA format, the vaccines of the invention can encode HA antigens, and optionally corresponding NA antigens, of circulating strains/lineages that represent multiple, distinct influenza clades and sub-clades, producing vaccines more efficacious at combatting an upcoming or forthcoming influenza season.

### Coronaviridae Family

The Coronaviridae family comprises enveloped, positive-strand RNA viruses which infect mammals, amphibians, and birds. A Coronaviridae subfamily, Orthocoronavirinae includes RNA viruses that cause disease in mammals and birds, causing respiratory tract infections ranging from the common cold to more lethal diseases (e.g., SARS, MERS, COVID-19). In some embodiments, a respiratory virus antigenic polypeptide is from a genus of Betacoronavirus, for example: MERS-CoV, SARS-CoV (SARS-CoV-1), SARS-CoV-2, HCoV-OC43, HCoV-229E, HCoV-NL63, HCoV-NL, HCoV-NH, or HCoV-HKU1.

The genome of Severe Acute Respiratory Syndrome Coronavirus 2 (SARS-CoV-2) is a single-stranded positive-sense RNA (+ssRNA) with the size of 29.8-30 kb encoding about 9860 amino acids (Chan et al. 2000, supra; Kim et al. 2020 Cell, May 14; 181(4):914-921.e10.). SARS-CoV-2 is a polycistronic mRNA with 5′-cap and 3′-poly-A tail. The SARS-CoV-2 genome is organized into specific genes encoding structural proteins and nonstructural proteins (Nsps). The order of the structural proteins in the genome is 5′-replicase (open reading frame (ORF)1/ab)-structural proteins [Spike (S)-Envelope (E)-Membrane (M)-Nucleocapsid (N)]-3′. The genome of coronaviruses includes a variable number of open reading frames that encode accessory proteins, nonstructural proteins, and structural proteins (Song et al. 2019 Viruses;11(1):p. 59). Most of the antigenic peptides are located in the structural proteins (Cui et al. 2019 Nat. Rev. Microbiol.; 17(3):181-192). Spike surface glycoprotein (S), a small envelope protein (E), matrix protein (M), and nucleocapsid protein (N) are four main structural proteins. Since S-protein contributes to cell tropism and virus entry and also it is capable to induce neutralizing antibodies (NAb) and protective immunity, it can be considered one of the most important targets in coronavirus vaccine development among all other structural proteins.

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids.

mRNAs of the invention are designed to produce SARS-CoV-2 Spike proteins (i.e., encode Spike proteins such that Spike protein is expressed when the mRNA is delivered to a cell or tissue, for example a cell or tissue in a subject), as well as structurally altered antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. In some embodiments, the Spike protein is not a stabilized Spike protein, for example, the Spike protein is stabilized by two proline substitutions (a 2P mutation).

In some embodiments, the Spike protein is from a different virus strain. A strain is a genetic variant of a microorganism (e.g., a virus). New viral strains can be created due to mutation or swapping of genetic components when two or more viruses infect the same cell in nature, for example, by antigenic drift or antigenic shift.

Antigenic drift is a kind of genetic variation in viruses, arising by the accumulation of mutations in the virus genes that code for virus-surface proteins that host antibodies recognize. This results in a new strain of virus particles that is not effectively inhibited by the antibodies that prevented infection by previous strains. This makes it easier for the changed virus to spread throughout a partially immune population.

Antigenic shift is the process by which two or more different strains of a virus, or strains of two or more different viruses, combine to form a new subtype having a mixture of the surface antigens of the two or more original strains. The term is often applied specifically to influenza, as that is the best-known example, but the process is also known to occur with other viruses. Antigenic shift is a specific case of reassortment or viral shift that confers a phenotypic change. Antigenic shift is contrasted with antigenic drift, which is the natural mutation over time of known strains of a virus which may lead to a loss of immunity, or in vaccine mismatch. Antigenic shift is often associated with a major reorganization of viral surface antigens, resulting in a reassortment change the virus's phenotype drastically.

A virus strain as used herein is a genetic variant or of a virus that is characterized by a mutation one or more surface proteins or other proteins of the virus. In the case of SARS-CoV-2, for example, a different amino acid sequence in the SARS-CoV-2 spike protein where the immune response in an individual to the new strain is less effective than to the strain used to immunize or first infect the individual. A new virus strain may arise from natural mutation or a combination of natural mutation and immune selection due to an ongoing immune response in an immunized or previously infected individual. A new virus strain can differ by one, two, three or more amino acid mutations in regions of the spike protein responsible for a viral function such as receptor binding or viral fusion with a target cell. A spike protein from a new strain may differ from the parental strain by as much as 80%, 85%, 90%, 95%, 98%, 99% identity at the amino acid level.

A natural virus strain is a variant of a given virus that is recognizable because it possesses some “unique phenotypic characteristics” that remain stable (e.g., stable and heritable biological, serological, and/or molecular characters) under natural conditions. Such “unique phenotypic characteristics” are biological properties different from the compared reference virus, such as unique antigenic properties, host range (e.g., infecting a different kind of host), symptoms of disease caused by the strain, different type of disease caused by the strain (e.g., transmitted by different means), etc. A “unique phenotypic characteristic” can be detected clinically (e.g., clinical manifestations detected in a host infected with the strain) or within a comparative animal experiment in which a researcher skilled in the art of virology can distinguish between the reference control virus-infected animal and the animal infected with the alleged new strain, without knowing which animal received which virus and without having any information about the differences between the two viruses. Importantly, a virus variant with a simple difference in genome sequence is not a separate strain if there is no recognizable distinct viral phenotype. The extent of genomic sequence variation is irrelevant for the classification of a variant as a strain since a distinct phenotype sometimes arises from few mutations.

As an example, in some embodiments, the mRNA encodes an antigen from at least one virus strain variant or comprises mutations from at least one virus strain that is not wild-type SARS-CoV-2. In some embodiments, the vaccine comprises mRNA encoding a Spike protein associated with the B.1.1.7 lineage (UK) variant (20B/501Y.V1 VOC 202012/01). The B.1.1.7 lineage variant has a mutation in the receptor binding domain (RBD) of the Spike protein at position 501, where amino acid asparagine (N) has been replaced with tyrosine (Y); an N501Y mutation. Further, the variant has a 69/70 deletion, which occurs spontaneously numerous times, leading to conformation changes in the Spike protein, a P681H mutation near the S1/S2 furin cleavage site, and a ORF8 stop codon (Q27 stop) caused by a mutation in ORF8. The 501.V2 (South Africa, SA) variant comprises multiple mutations in the Spike protein, including N501Y, and E484K, but does not have a deletion at 69/70. The E484K mutation is considered to be an “escape” mutation relative to at least one form of monoclonal antibody against SARS-CoV-2, such that it may change the antigenicity of the virus. Other mutations that have been discovered include the D614G mutation, which is thought to increase the transmission rate of the virus, and the N543Y mutation (emerged from mink farms in the Netherlands and Denmark). In some embodiments, the Spike protein comprises mutations from more than one variant (e.g., a combination of mutations found in the B.1.1.7 and 502Y.V2 variants) and is a structurally altered variant having multiple mutations.

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, specifically angiotensin-converting enzyme 2 (ACE2) receptors, whereas the S2 subunit, which is the most conserved component of the S protein, will be responsible for fusing the envelope of the virus with the host cell membrane. (See e.g., Shang et al., PLoS Pathog. 2020 March; 16(3):e1008392.). Each monomer of trimeric S protein trimer contains the two subunits, S1 and S2, mediating attachment and membrane fusion, respectively. As part of the infection process in vivo, the two subunits are separated from each other by an enzymatic cleavage process. S protein is first cleaved by furin-mediated cleavage at the S1/S2 site in infected cells, In vivo, a subsequent serine protease-mediated cleavage event occurs at the S2′ site within S1. In SARS-CoV2, the S1/S2 cleavage site is at amino acids 676-TQTNSPRRAR/SVA—688 (referencing SEQ ID NO: 49). The S2′ cleavage site is at amino acids 811—KPSKR/SFI-818 (referencing SEQ ID NO: 50).

As used herein, for example in the context of designing SARS-CoV-2 S protein antigens encoded by the nucleic acids, e.g., mRNAs, of the invention, the term “S1 subunit” (e.g., S1 subunit antigen) refers to the N-terminal subunit of the Spike protein beginning at the S protein N-terminus and ending at the S1/S2 cleavage site whereas the term “S2 subunit” (e.g., S2 subunit antigen) refers to the C-terminal subunit of the Spike protein beginning at the S1/S2 cleavage site and ending at the C-terminus of the Spike protein. As described supra, the skilled artisan will understand that, while an essentially full length or complete Spike protein S1 or S2 subunit may be necessary for receptor binding or membrane fusion, respectively, a certain amount of variation in S1 or S2 structure and/or sequence is tolerated when seeking primarily to elicit an immune response against Spike protein subunits. For example, minor truncation, e.g., of one to a few, possibly up to 4, 5, 6, 7, 8, 9 or 10 amino acids from the N- or C-terminus of the encoded subunit, e.g., encoded S1 or S2 protein antigens, may be tolerated without changing the antigenic properties of the protein. Likewise, variation (e.g., conservative substitution) of one to a few, possibly up to 4, 5, 6, 7, 8, 9 or 10 amino acids (or more) of the encoded Spike protein subunits, e.g., encoded S1 or S2 protein antigen, may be tolerated without changing the antigenic properties of the protein(s).

The S1 and S2 subunits of the SARS-CoV-2 Spike protein further include domains readily discernable by structure and function, which in turn can be featured in designing antigens to be encoded by the nucleic acid vaccines, in particular, mRNA vaccines of the invention. Within the S1 subunit, domains include the N-terminal domain (NTD) and the receptor-binding domain (RBD), said RBD domain further including a receptor-binding motif (RBM) Within the S2 subunit, domains include fusion peptide (FP), heptad repeat 1 (HR1), heptad repeat 2 (HR2), transmembrane domain (TM), and cytoplasm domain, also known as cytoplasmic tail (CT) (Lu R. et al., supra; Wan et al., J. Virol. Mar 2020, 94 (7) e00127-20). The HR1 and HR2 domains can be referred to as the “fusion core region” of SARS-CoV-2 (Xia et al., 2020 Cell Mol Immunol. Jan; 17(1):1-12.). The S1 subunit includes an N terminal domain (NTD), a linker region, a receptor binding domain (RBD), a first subdomain (SD1), and a second subdomain (SD2). The S2 subunit includes, inter alia, a first heptad repeat (HR1), a second heptad repeat (HR2), a transmembrane domain (TM), and a cytoplasmic tail. The NTD and RBD of S1 are good antigens for the vaccine design approach of the invention as these domains have been shown to be the targets of neutralizing antibodies in betacoronavirus-infected individuals.

The compositions provided herein include mRNA that may encode any one or more full-length or partial (truncated or other deletion of sequence) S protein subunit (e.g., S1 or S2 subunit), one or more domain or combination of domains of an S protein subunit (e.g., NTD, RBD, or NTD-RBD fusions, with or without an SD1 and/or SD2), or chimeras of full-length or partial and S2 protein subunits. Other S protein subunit and/or domain configurations are contemplated herein. Exemplary SARS-CoV-2 mRNA vaccines are provided in PCT/US2021/015145 and PCT/US2021/016979, each incorporated herein by reference in its entirety.

The genome of SARS-CoV (e.g., SARS-CoV-1) also includes of a single, positive-strand RNA that is approximately 29,700 nucleotides long. The overall genome organization of SARS-CoV is similar to that of other coronaviruses. The reference genome includes 13 genes, which encode at least 14 proteins. Two large overlapping reading frames (ORFs) encompass 71% of the genome. The remainder has 12 potential ORFs, including genes for structural proteins S (spike), E (small envelope), M (membrane), and N (nucleocapsid). Other potential ORFs code for unique putative SARS-CoV-specific polypeptides that lack obvious sequence similarity to known proteins. A detailed analysis of the SARS-CoV genome has been published in J Mol Biol 2003; 331: 991-1004.

In some embodiments, a vaccine of the present disclosure comprises an RNA (e.g., mRNA) polynucleotide encoding a SARS-CoV S protein. In some embodiments, a vaccine of the present disclosure comprises an RNA (e.g., mRNA) polynucleotide encoding the S1 subunit of the SARS-CoV S protein. In some embodiments, a vaccine of the present disclosure comprises an RNA (e.g., mRNA) polynucleotide encoding the S2 subunit of the SARS-CoV S protein. In some embodiments, a vaccine of the present disclosure comprises an RNA (e.g., mRNA) polynucleotide encoding a SARS-CoV E protein. In some embodiments, a vaccine of the present disclosure comprises an RNA (e.g., mRNA) polynucleotide encoding a SARS-CoV N protein. In some embodiments, a vaccine of the present disclosure comprises an RNA (e.g., mRNA) polynucleotide encoding a SARS-CoV M protein. In some embodiments, a vaccine of the present disclosure comprises an RNA (e.g., mRNA) polynucleotide encoding at least one of the following SARS-CoV proteins: S protein (S, S1 and/or S2), E protein, N protein and M protein.

MERS-CoV is a positive-sense, single-stranded RNA virus of the genus Betacoronavirus. The genomes are phylogenetically classified into two clades, clade A and clade B. The genome of MERS-CoV encodes at least four unique accessory proteins, such as 3, 4a, 4b and 5, two replicase proteins (open reading frame 1a and 1b), and four major structural proteins, including spike (S), envelope (E), nucleocapsid (N), and membrane (M) proteins (Almazan F et al. MBio 2013;4(5):e00650-13). The S protein is particularly essential in mediating virus binding to cells expressing receptor dipeptidyl peptidase-4 (DPP4) through receptor-binding domain (RBD) in the S1 subunit, whereas the S2 subunit subsequently mediates virus entry via fusion of the virus and target cell membranes (Li F. J Virol 2015; 89(4):1954-64; Raj V S et al. Nature 2013; 495(7440):251-4).

In some embodiments, a vaccine of the present disclosure comprises an RNA (e.g., mRNA) polynucleotide encoding a MERS-CoV S protein. In some embodiments, a vaccine of the present disclosure comprises an RNA (e.g., mRNA) polynucleotide encoding the S1 subunit of the MERS-CoV S protein. In some embodiments, a vaccine of the present disclosure comprises an RNA (e.g., mRNA) polynucleotide encoding the S2 subunit of the MERS-CoV S protein. In some embodiments, a vaccine of the present disclosure comprises an RNA (e.g., mRNA) polynucleotide encoding a MERS-CoV E protein. In some embodiments, a vaccine of the present disclosure comprises an RNA (e.g., mRNA) polynucleotide encoding a MERS-CoV N protein. In some embodiments, a vaccine of the present disclosure comprises an RNA (e.g., mRNA) polynucleotide encoding a MERS-CoV M protein. In some embodiments, a vaccine of the present disclosure comprises an RNA (e.g., mRNA) polynucleotide encoding at least one of the following MERS-CoV proteins: S protein (S, S1 and/or S2), E protein, N protein and M protein.

Human coronavirus OC43 is an enveloped, positive-sense, single-stranded RNA virus in the species Betacoronavirus-1 (genus Betacoronavirus, subfamily Coronavirinae, family Coronaviridae, order Nidovirales). Four HCoV-OC43 genotypes (A to D), have been identified with genotype D most likely arising from recombination. Along with HCoV-229E, a species in the Alphacoronavirus genus, HCoV-OC43 are among the known viruses that cause the common cold. Both viruses can cause severe lower respiratory tract infections, including pneumonia in infants, the elderly, and immunocompromised individuals such as those undergoing chemotherapy and those with HIV-AIDS. In some embodiments, a vaccine of the present disclosure comprises an RNA (e.g., mRNA) polynucleotide encoding an HCoV-OC43 protein.

Human coronavirus HKU1 (HCoV-HK U1) is a positive-sense, single-stranded RNA virus with the HE gene, which distinguishes it as a group 2, or betacoronavirus. The genome organization is the same as that of other group II coronaviruses, with the characteristic gene order 1a, 1b, HE, S, E, M, and N. Furthermore, accessory protein genes are present between the S and E genes (ORF4) and at the position of the N gene (ORF8). The TRS is presumably located within the AAUCUAAAC sequence, which precedes each ORF except E. In some embodiments, a vaccine of the present disclosure comprises an RNA (e.g., mRNA) polynucleotide encoding an HKU1 HE protein. In some embodiments, a vaccine of the present disclosure comprises an RNA (e.g., mRNA) polynucleotide encoding an HKU1 S protein. In some embodiments, a vaccine of the present disclosure comprises an RNA (e.g., mRNA) polynucleotide encoding an HKUI1 E protein. In some embodiments, a vaccine of the present disclosure comprises an RNA (e.g., mRNA) polynucleotide encoding an 1HIKU1 M protein. In some embodiments, a vaccine of the present disclosure comprises an RNA (e.g., mRNA) polynucleotide encoding an HKU1 N protein. In some embodiments, a vaccine of the present disclosure comprises an RNA (e.g., mRNA) polynucleotide encoding at least one of the following HKU1 proteins: HE protein, S protein, E protein, N protein and M protein.

In some embodiments, the betacoronavirus is human coronavirus NL63 (HCoV-NL63 or HCoV-NL). Human New Haven coronavirus, HCoV-NH, is a strain of human coronavirus NL63. Genes predicted to encode the S, E, M, and N proteins are found in the 3′ part of the HCoV-NL63 genome. In some embodiments, a vaccine of the present disclosure comprises an RNA (e.g., mRNA) polynucleotide encoding an NL63 S protein. In some embodiments, a vaccine of the present disclosure comprises an RNA (e.g., mRNA) polynucleotide encoding an NL63 S protein. In some embodiments, a vaccine of the present disclosure comprises an RNA (e.g., mRNA) polynucleotide encoding an 1-1 NL63 KU1 E protein. In some embodiments, a vaccine of the present disclosure comprises an RNA (e.g., mRNA) polynucleotide encoding an NL63 M protein. In some embodiments, a vaccine of the present disclosure comprises an RNA (e.g., mRNA) polynucleotide encoding an NL63 N protein. In some embodiments, a vaccine of the present disclosure comprises an RNA (e.g., mRNA) polynucleotide encoding at least one of the following NL63 proteins: S protein, E protein, N protein and M protein.

Human coronavirus 229E (HCoV-229E) is a single-stranded, positive-sense, RNA virus species in the Alphacoronavirus genus of the subfamily Coronavirinae, in the family Coronaviridae, of the order Nidovirales. Along with Human coronavirus OC43, it is responsible for the common cold. In some embodiments, a vaccine of the present disclosure comprises an RNA (e.g., mRNA) polynucleotide encoding an HCoV-229E antigenic protein.

It will be understood to those of skill in the art that viral classification evolves as additional viruses are identified and sequenced. While specific examples of respiratory viruses involved in human disease are set forth and exemplified herein, the mRNA vaccines of the invention can include other human respiratory viruses, e.g., viruses in these families or related human respiratory viruses that are not specifically set forth. To the extent that viruses are specifically identified herein as falling within a specific family or subfamily, those viruses are explicitly noted to be within those families/subfamilies even if they are later reclassified or are identified differently or inconsistently in other publications or sources. It will be understood that if a virus was in the past, is currently, or is in the future, classified under one of the families, subfamilies, or genera described or claimed herein, it is considered to fall within the scope of that viral family, subfamily or genus as those terms are defined and used herein.

Embodiments of the present disclosure provide combination vaccines (e.g., combination mRNA vaccines). A “combination vaccine” of the present disclosure refers to a vaccine comprising at least 2 polynucleotides, each comprising an open reading frame encoding at least one respiratory virus antigenic polypeptide, wherein there is at least one polynucleotide encoding an influenza antigen and at least one polynucleotide encoding a coronavirus antigen. In another embodiment, the antigenic polypeptide is derived from the viral surface receptor binding glycoproteins, or proteins of the included viruses because these lead to inducing the best neutralizing antibody responses. In some embodiments, the combination vaccine comprises 2-15 mRNA polynucleotides, for example, 2-4, 3-4, 3-5, 3-6, 3-7, 3-8, 3-9, 3-10, 3-11, 3-12, 3-13, 3-14, 3-15, 4-5, 4-6, 4-7, 4-8, 4-9, 4-10, 4-11, 4-12, 4-13, 4-14, 4-15, 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15,7-8,7-9,7-10,7-11,7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15 mRNA polynucleotides. In some embodiments, the combination vaccine comprises 2, 3, 4, 5, 6, 7, 8, 9, 10, 11, 12, 13, 14, or 15 mRNA polynucleotides. In a particular embodiment, all the RNAs encode viral surface proteins, e.g., glycoproteins, involved in receptor binding to facilitate viral entry into host cells.

In some embodiments, the vaccine comprises at least two mRNA polynucleotides encoding influenza virus antigenic polypeptides. In some embodiments, the vaccine comprises at least three mRNA polynucleotides encoding influenza virus antigenic polypeptides. In some embodiments, the vaccine comprises at least four mRNA polynucleotides encoding influenza virus antigenic polypeptides. In some embodiments, the vaccine comprises at least 5, 6, 7, 8, 9, 10, 11, or 12 mRNA polynucleotides encoding influenza virus antigenic polypeptides.

In some embodiments, the vaccine comprises at least two mRNA polynucleotides encoding coronavirus antigenic polypeptides. In some embodiments, the vaccine comprises at least 2, 3, 4, 5, or 6 mRNA polynucleotides encoding coronavirus antigenic polypeptides.

In some embodiments, the mRNAs encoding the influenza antigens are present in the formulation in an equal amount (e.g., a 1:1 ratio), for example, a 1:1 ratio of mRNAs encoding distinct HA antigens, or a 1:1 ratio of mRNAs encoding distinct HA and NA antigens. In an exemplary vaccine comprising mRNAs encoding four different HA antigens, mRNAs at a “1:1 ratio” would include the mRNAs in a ratio of 1:1:1:1 of the first, second, third and fourth mRNA.

In an exemplary vaccine comprising mRNAs encoding four different HA antigens and four different NA antigens, mRNAs at a “1:1 ratio” would include the mRNAs encoding the different HA antigens in a ratio of 1:1:1:1 of the first, second, third and fourth mRNA, and would include mRNAs encoding the different NA antigens in a ratio of 1:1:1:1 of the first, second, third and fourth mRNA.

In some embodiments, the ratio of mRNAs encoding the different HA antigens are equivalent to each other (e.g., 1:1:1:1) and the ratio of mRNAs encoding the different NA antigens are equivalent to each other (e.g., 1:1:1:1); however, the ratio of the mRNAs encoding the HA antigens to mRNAs encoding the NA antigens is not 1:1. In an exemplary vaccine comprising mRNAs encoding four different HA antigens and four different NA antigens, mRNAs at a “3:1 ratio” would include the mRNAs encoding the different HA antigens in a ratio of 3:3:3:3 of the first, second, third and fourth mRNA, and would include mRNAs encoding the different NA antigens in a ratio of 1:1:1:1 of the first, second, third and fourth mRNA. In some embodiments, the HA:NA ratio is 1:1, 1:2, 1:3, 1:4, 2:1, 3:1, or 4:1.

In some embodiments, the first and second mRNA polynucleotides are present in the combination vaccine in a ratio of 1:1 (e.g., flu mRNA polynucleotide:coronavirus mRNA polynucleotide). In some embodiments, the combination vaccine comprises a ratio of mRNA polynucleotides encoding respiratory virus antigenic polypeptides of 4:1 from the first virus (e.g., influenza) to the second virus. In some embodiments, the combination vaccine comprises a ratio of mRNA polynucleotides encoding respiratory virus antigenic polypeptides of 3:1 from the first virus (e.g., influenza) to the second virus (e.g., coronavirus). In some embodiments, the combination vaccine comprises a ratio of mRNA polynucleotides encoding respiratory virus antigenic polypeptides of 2:1 from the first virus (e.g., influenza) to the second virus (e.g., coronavirus). In some embodiments, the combination vaccine comprises a ratio of mRNA polynucleotides encoding respiratory virus antigenic polypeptides of 5:1 from the first virus (e.g., influenza) to the second virus (e.g., coronavirus). In some embodiments, the combination vaccine comprises a ratio of mRNA polynucleotides encoding respiratory virus antigenic polypeptides of 1:2 from the first virus (e.g., influenza) to the second virus (e.g., coronavirus). In some embodiments, the combination vaccine (e.g., multivalent RNA composition) comprises a ratio of mRNA polynucleotides encoding respiratory virus antigenic polypeptides of 4:1, 4:2, 4:3, 1:4, 2:4, or 3:4 from the first virus to the second virus (e.g., coronavirus).

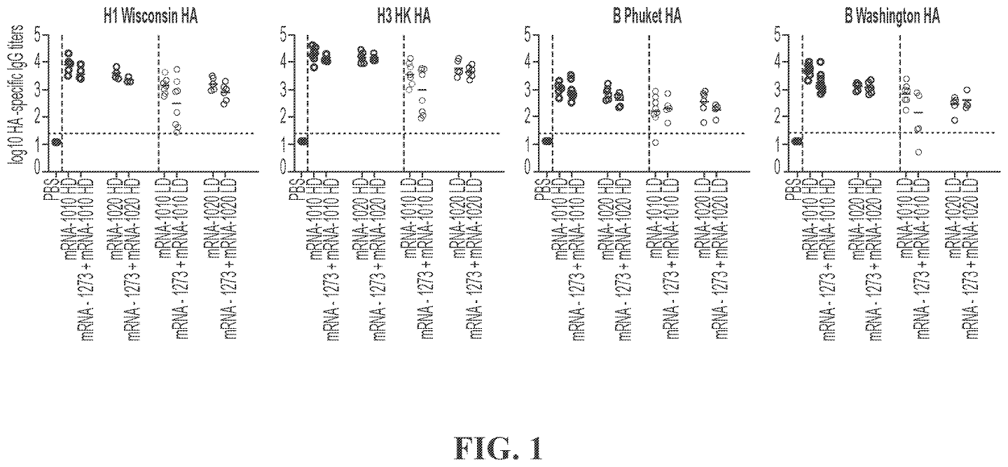

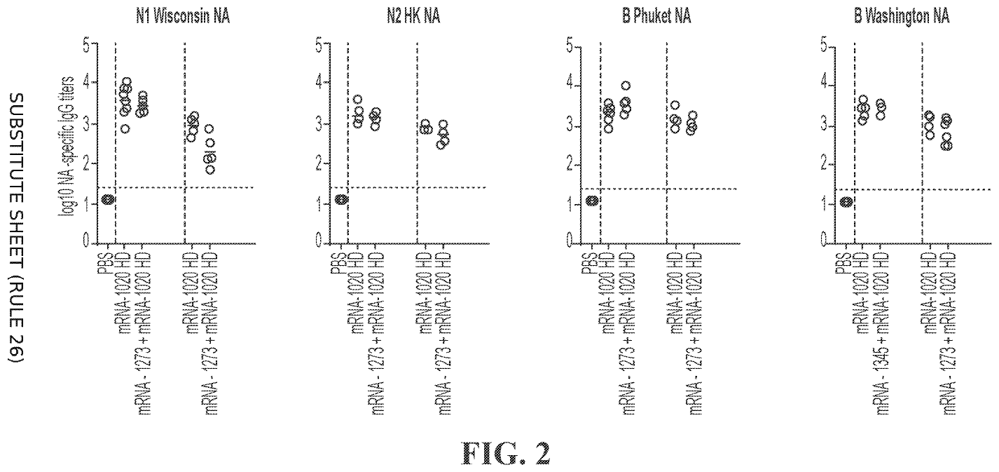

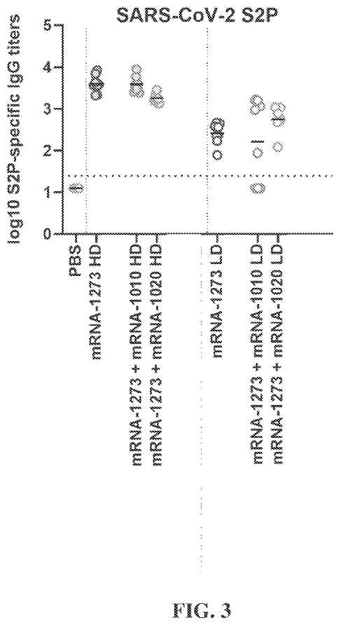

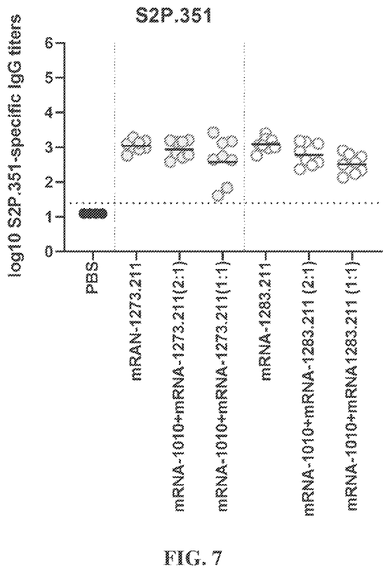

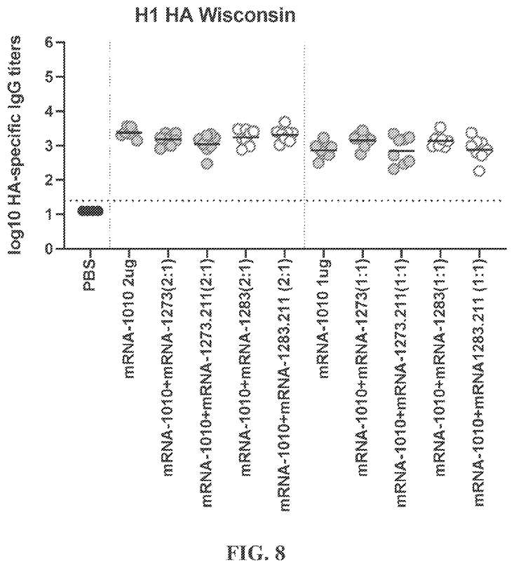

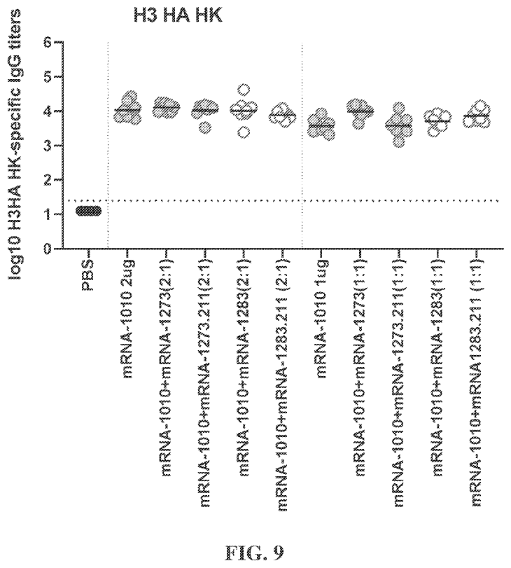

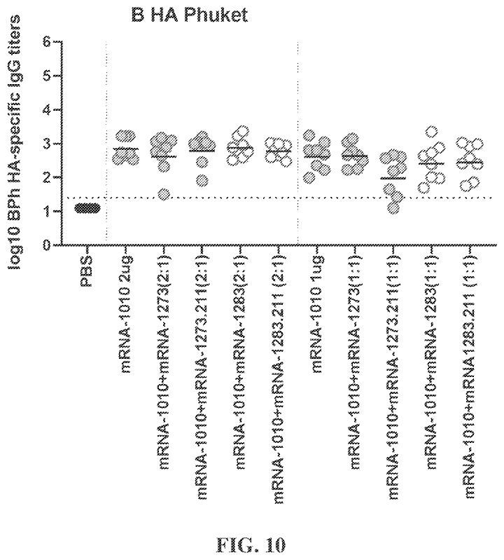

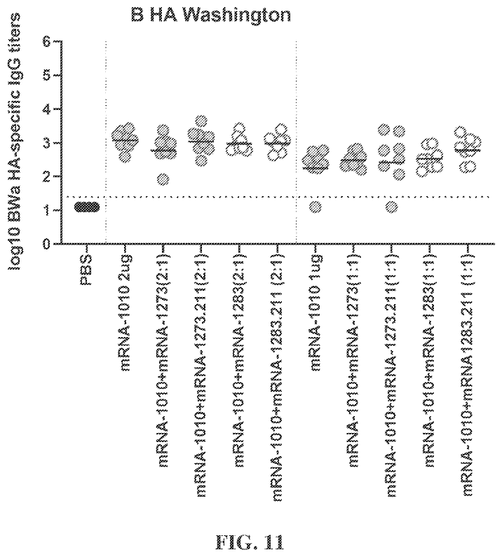

In some embodiments, each of the mRNA polynucleotides in the combination vaccine is complementary with i.e., does not interfere with each other mRNA polynucleotide in the combination vaccine. That is, an antigen produced from administration of the combination vaccine does not significantly interfere with the immune response to any other of the antigens produced in response to the vaccine in such a way that would diminish the ability of the antigens to provoke a protective immune response in a subject. In some embodiments, the combination vaccine is additive with respect to neutralizing antibodies relative to each individual antigen in a vaccine. As is shown in FIGS. 1-11, administration of combination vaccines comprising mRNA polynucleotides encoding influenza antigens and SARS-CoV-2 antigen did not inhibit or reduce the neutralizing antibody titers for each respective antigen relative to administration of mRNA encoding each single antigen separately.

In each embodiment or aspect of the invention, it is understood that the featured vaccines include the mRNAs encapsulated within LNPs. While it is possible to encapsulate each unique mRNA in its own LNP, the mRNA vaccine technology enjoys the significant technological advantage of being able to encapsulate several mRNAs in a single LNP product.

Nucleic Acids The compositions of the present disclosure comprise a (at least one) messenger RNA (mRNA) having an open reading frame (ORF) encoding an influenza virus antigen and a coronavirus antigen. In some embodiments, the mRNA further comprises a 5′ UTR, 3′ UTR, a poly(A) tail and/or a 5′ cap analog.

In some embodiments, the first, second and/or third mRNA polynucleotides in the composition differ in length from one another by at least 100 nucleotides (e.g., 100, 110, 120, 130, 140, 150, 160, 170, 180, 190, 200, or more nucleotides).

It should also be understood that the respiratory virus vaccine of the present disclosure may include any 5′ untranslated region (UTR) and/or any 3′ UTR. Exemplary UTR sequences include SEQ ID NOs: 29-32; however, other UTR sequences may be used or exchanged for any of the UTR sequences described herein. In some embodiments, a 5′ UTR of the present disclosure comprises a sequence selected from SEQ ID NO: 29 (GGGAAAUA AGAGAGAAAAGAAGAGUAAGAAGAAAUAUAAGAGCCACC) and SEQ ID NO: 30 (GGGAAAUAAGAGAGAAAAGAAGAGUAAGAAGAAAUAUAAGACCCCGGCGCCGCC ACC). In some embodiments, a 3′ UTR of the present disclosure comprises a sequence selected from SEQ ID NO: 31 (UGAUAAUAGGCUGGAGCCUCGGUGGCCAUGCUU CUUGCCCCUUGGGCCUCCCCCCAGCCCCUCCUCCCCUUCCUGCACCCGUACCCCCG UGGUCUUUGAAUAAAGUCUGAGUGGGCGGC) and SEQ ID NO: 32 (UGAUAA UAGGCUGGAGCCUCGGUGGCCUAGCUUCUUGCCCCUUGGGCCUCCCCCCAGCCCC UCCUCCCCUUCCUGCACCCGUACCCCCGUGGUCUUUGAAUAAAGUCUGAGUGGGC GGC). UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a P-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

### Variants

In some embodiments, the compositions of the present disclosure include RNA that encodes a respiratory virus antigens and structurally altered variants representing a plurality of virus antigens. Antigenic variants or structurally altered variants refers to molecules that differ in their amino acid sequence from a wild-type (naturally occurring), native, or reference protein sequence. The antigen/structurally altered variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, vary the breadth of their immunogenicity, i.e. with respect to breadth of immune response generated, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein, or comprises a nucleotide sequence at least 90%, at least 95%, at least 96%, at least 97%, at least 98%, or at least 99% identical to a nucleotide sequence of a wild-type (naturally occurring) or variant antigen.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C.D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding proteins or glycoproteins containing substitutions, insertions and/or additions, deletions, and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of respiratory virus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to a respiratory virus.

In addition to structurally altered variants that are identical to the reference protein but are truncated, in some embodiments, a structurally altered variant includes an antigen that has 1, 2, 3, 4, 5, 6, 7, 8, 9, 10, or more mutations with respect to a reference antigen. Some examples of structurally altered variants are shown in the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA. In some embodiments, the combination vaccine (e.g., multivalent RNA composition) comprises greater than 20%, 30%, 40%, 50%, or 60% polyA-tailed RNAs.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, P-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of 20 approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to a respiratory virus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than respiratory virus antigens in nature) are known in the art and can be tested for desired properties and then incorporated into a nucleic acid of the disclosure.

### Fusion Proteins

In some embodiments, a composition of the present disclosure includes an mRNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the respiratory virus antigen. Antigenic fusion proteins, in some embodiments, retain the functional property from each original protein.

### Scaffold Moieties

The mRNA vaccines as provided herein, in some embodiments, encode fusion proteins that comprise respiratory virus antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300A and 360A diameter, corresponding to 180 or 240 protomers. In some embodiments, the respiratory virus antigen is fused to HBsAG or HBcAG to facilitate self-assembly of nanoparticles displaying the respiratory virus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S.E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150A diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, an RNA of the present disclosure encodes respiratory virus antigen fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers,T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a respiratory virus antigen). In some embodiments, a codon optimized sequence shares less than 90% sequence identity to a naturally-occurring or wild-type sequence (e.g., a naturally-occurring or wild-type mRNA sequence encoding a respiratory virus antigen). In some embodiments, a codon optimized sequence shares less than 85% sequence identity to a naturally-occurring or wild-type sequence (e.g., a naturally-occurring or wild-type mRNA sequence encoding a respiratory virus antigen). In some embodiments, a codon optimized sequence shares less than 80% sequence identity to a naturally-occurring or wild-type sequence (e.g., a naturally-occurring or wild-type mRNA sequence encoding a respiratory virus antigen). In some embodiments, a codon optimized sequence shares less than 75% sequence identity to a naturally-occurring or wild-type sequence (e.g., a naturally-occurring or wild-type mRNA sequence encoding a a respiratory virus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a respiratory virus antigen). In some embodiments, a codon optimized sequence shares between 65% and 75% or about 80% sequence identity to a naturally-occurring or wild-type sequence (e.g., a naturally-occurring or wild-type mRNA sequence encoding a respiratory virus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a respiratory virus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a respiratory virus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/036773; PCT/US2015/036759; PCT/US2015/036771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (elyl), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (W) substitutions at one or more or all uridine positions of the nucleic acid.

In some embodiments, a mRNA of the disclosure comprises pseudouridine (W) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 68), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 48) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 29 and SEQ ID NO:30.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif, c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3” UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

In vitro Transcription of RNA

Aspects of the present disclosure provide methods of producing (e.g., synthesizing) a RNA transcript (e.g., mRNA transcript) comprising contacting a DNA template (e.g., a first input DNA and a second input DNA) with a RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

In some embodiments, a wild-type T7 polymerase is used in an IVT reaction. In some embodiments, a modified or mutant T7 polymerase is used in an IVT reaction. In some embodiments, a T7 RNA polymerase variant comprises an amino acid sequences that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) tail located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

Multivalent mRNA constructs are typically produced by transcribing one mRNA product at a time, purifying each mRNA product, and then mixing the purified mRNA products together prior to formulation. This type of process incurs significant time and monetary investment especially at the Good Manufacturing Practice (GMP) scale.

Aspects of the disclosure relate to methods for producing compositions comprising multivalent different RNAs (e.g., 2 or more different RNAs). In some aspects, methods of multivalent transcription disclosed herein involve selecting amounts of input DNA for IVT reactions that result in multivalent RNA compositions having higher purity than RNA compositions produced using previous methods. It was observed that certain characteristics or properties of DNA molecules being co-transcribed (e.g., transcribed simultaneously in vitro), such as differences in length between DNA molecules, polyA-tailing efficiency of DNA molecules, etc., and/or other reagents present in the co-IVT reaction mixture (e.g., RNA polymerase, nucleotide triphosphates (NTPs), etc.) may introduce compositional bias into the resulting multivalent RNA compositions. Surprisingly, methods were discovered that reduce such compositional bias. In some embodiments, modifying input DNA amounts results in production of multivalent RNA compositions having increased purity (e.g., as measured by percentage of RNAs comprising polyA tails) relative to RNA compositions produced by previous methods. It was also surprisingly discovered that co-IVT methods described herein result in high purity multivalent RNA compositions even when there is a large difference (e.g., >100 nucleotides) in the lengths of the input DNAs used in the IVT reaction.

Accordingly, in some aspects, the disclosure provides a method for producing a multivalent RNA composition, the method comprising simultaneously in vitro transcribing at least two DNA molecules in a reaction mixture comprising: a first population of DNA molecules encoding a first RNA; a second population of DNA molecules encoding a second RNA that is different than the first RNA; and obtaining a multivalent RNA composition having a pre-defined ratio of the first RNA to the second RNA produced by the IVT.

As used herein, the term “multivalent RNA composition” refers to a composition comprising more than two different mRNAs. A multivalent RNA composition may comprise 2 or more different RNAs, for example 2, 3, 4, 5, 6, 7, 8, 9, 10, or more different RNAs. In some embodiments, a multivalent RNA composition comprises more than 10 different RNAs. The term “different RNAs” refers to any RNA that is not the same as another RNA in a multivalent RNA composition. For example, two RNAs are different if they have 1) different lengths (whether or not the RNAs are identical over the entirety of the shorter of the two lengths), ii) different nucleotide sequences, iii) different chemical modification patterns, or iv) any combination of the foregoing.

In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). Methods of obtaining populations of input DNAs (e.g., plasmid DNAs) are known, for example as described by Sambrook, Joseph. Molecular Cloning: a Laboratory Manual. Cold Spring Harbor, N.Y.: Cold Spring Harbor Laboratory Press, 2001.

Some aspects comprise normalizing the amount of DNA used in the multivalent co-IVT reaction. In some embodiments, the normalization is based on the molar mass of the input DNAs. In some embodiments, the normalization is based on the degradation rate of the input DNAs. In some embodiments, the normalization is based on the degradation rate of the resultant mRNAs (e.g., measured based upon polyA variants present in the reaction mixture, or T7 polymerase abortive transcripts or truncated transcripts). In some embodiments, the normalization is based on the nucleotide content (e.g., amount of A, G, C, U, or any combination thereof) of the input DNAs. In some embodiments, the normalization is based on the purity of the input DNAs. In some embodiments the normalization is based on the polyA-tailing efficiency of the input DNAs. In some embodiments, the normalization is based on the lengths of the input DNAs.

In some embodiments, mRNA is at a pre-defined mRNA ratio, which may comprise a ratio between 2, 3, 4, 5, 6, 7, 8, 9, 10, or more different RNAs (e.g., depending on the number of different RNAs in a composition). In some embodiments, a pre-defined ratio comprises a ratio between more than 10 RNAs. As used herein, a “pre-defined mRNA ratio” refers to the desired final ratio of RNA molecules in a multivalent RNA composition. The desired final ratio of an RNA composition will depend upon the final peptide(s) or polypeptide product(s) encoded by the RNAs. For example, a multivalent RNA mixture may comprise two RNAs (e.g., a RNA encoding a first antigen and a second antigen); in this instance the desired final ratio of RNA molecules may be 1 first antigen RNA:1 second antigen RNA. In another example, a multivalent RNA composition may comprise several (e.g., 3, 4, 5, 6, 7, 8, or more) RNAs encoding different antigenic peptides (e.g., for use as a vaccine); in that instance the desired ratio may comprise between 3 and 10 RNAs (e.g., a:b:c, a:b:c:d, a:b:c:d:e, a:b:c:d:e:f, a:b:c:d:e:f:g, a:b:c:d:e:f:g:h, a:b:c:d:e:f:g:h:i, a:b:c:d:e:f:g:h:i:j, etc., where each of a-j is a number between 1 and 10).

In some embodiments, the normalization is based on the lowest level present in the input DNAs (e.g., lowest molar mass, degradation rate (e.g., of the input DNA and/or output RNA), nucleotide content, purity, and/or polyA-tailing efficiency). In some embodiments, the normalization is based on the highest level present in the input DNAs (e.g., highest molar mass, degradation rate (e.g., of the input DNA and/or output RNA), nucleotide context, purity, and/or polyA-tailing efficiency). In some embodiments, the normalization is based on the rate of RNA production of the input DNAs (e.g., the highest rate of RNA production of an input DNA or the lowest rate of RNA production of an input DNA in a reaction mixture).

In some aspects, the disclosure relates to IVT methods in which the amount of input DNA (e.g., a first DNA or second DNA) is adjusted or normalized in order to improve production of multivalent RNA compositions having a pre-defined mRNA ratio of components.

As described herein, certain factors affecting multivalent RNA composition purity, such as large differences in size between input DNAs (e.g., a difference of more than 100, 200, 500, 1000, or more nucleotides in length) and/or polyA-tailing efficiency of a given DNA during IVT, may be addressed prior to the IVT by normalizing the amount of input DNA based upon one or more of those factors.

The number of input DNAs (e.g., populations of input DNA molecules) used in an IVT reaction may vary, depending upon the number of different RNA molecules desired to be included in the multivalent RNA composition. In some embodiments, an IVT reaction mixture comprises 2 or more different input DNAs, for example 2, 3, 4, 5, 6, 7, 8, 9, 10, 11, 12, 13, 14, 15, or more different input DNAs. In some embodiments, the IVT reaction comprises more than 15 different input DNAs. The term “different input DNAs” encompasses input DNAs that encode different RNAs, e.g., that have i) different lengths (whether or not the RNAs are identical over the entirety of the shorter of the two lengths), ii) different nucleotide sequences, iii) different chemical modification patterns, or iv) any combination of the foregoing.

In some embodiments, two or more of the input DNA molecules used in an IVT reaction encode mRNA molecules that have a different length (e.g., comprises a different number of nucleotides). In some embodiments, the difference in length between two or more of the mRNA molecules encoded by different input DNA molecules in an IVT reaction mixture is greater than 70 nucleotides, 80 nucleotides, 90 nucleotides, or 100 nucleotides (e.g., two input DNAs in a composition encode mRNA molecules that are not are within 70, 80, 90, or 100 nucleotides in length of one another). In some embodiments, the difference in length between two or more of the mRNA molecules encoded by different input DNA molecules is more than 100 nucleotides, for example 500 nucleotides, 1000 nucleotides, 1500 nucleotides, 2000 nucleotides, 3000 nucleotides, 4000 nucleotides, or more.

In specific embodiments, the combination vaccine (e.g., multivalent RNA composition) is produced by combining a linearized first DNA molecule encoding the first mRNA polynucleotide, a linearized second DNA molecule encoding the second mRNA polynucleotide, and a linearized third DNA molecule encoding the third mRNA polynucleotide into a single reaction vessel, wherein the first DNA molecule, the second DNA molecule, and the third DNA molecule are obtained from different sources. In some embodiments, the different sources are a first, second, and third bacterial cell culture and wherein the first, second and third bacterial cell culture are not co-cultured. In some embodiments, the different sources are a first, second, and third bacterial cell culture and wherein the first, second and third bacterial cell culture are co-cultured. In some embodiments, the amounts of the first, second and third DNA molecules present in the reaction mixture prior to the start of the in vitro transcription have been normalized.

In some embodiments, the linearized first DNA molecule, the linearized second DNA molecule and the linearized third DNA molecule are simultaneously in vitro transcribed to obtain the multivalent RNA composition.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Non-coding Sequences

Aspects of the disclosure relate to multivalent RNA compositions which comprise mRNAs, e.g., 2-15 mRNA polynucleotides each comprising a distinct open reading frame (ORF) encoding a respiratory virus antigenic polypeptide, wherein each mRNA polynucleotide comprises one or more non-coding sequences in an untranslated region (UTR) having unique identifier sequences or non-coding sequences. As used herein, “non-coding sequence” refers to a sequence of a biological molecule (e.g., nucleic acid, protein, etc.) that when combined with the sequence another biological molecule serves to identify the other biological molecule.

Typically, a non-coding sequence is a heterologous sequence that is incorporated within or appended to a sequence of a target biological molecule and utilized as a reference in order to identify a target molecule of interest. In some embodiments, a non-coding sequence is a sequence of a nucleic acid (e.g., a heterologous or synthetic nucleic acid) that is incorporated within or appended to a target nucleic acid and utilized as a reference in order to identify the target nucleic acid. In some embodiments, a non-coding sequence is of the formula (N)n. In some embodiments, n is an integer in the range of 5 to 20, 5 to 10, 10 to 20, 7 to 20, or 7 to 30.

In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, N are each nucleotides that are independently selected from A, G, T, U, and C, or analogues thereof. Thus, some embodiments comprise nucleic acids (e.g., mRNAs) that (i) have a target sequence of interest (e.g., a coding sequence (e.g., that encodes therapeutic peptide or therapeutic protein)); and (ii) comprises a unique non-coding sequence.

In some embodiments, one or more in vitro transcribed mRNAs comprise one or more non-coding sequences in an untranslated region (UTR), such as a 5′ UTR or 3′ UTR. Inclusion of a non-coding sequence in the UTR of an mRNA prevents non-coding sequence from being translated into a peptide. In some embodiments, a non-coding sequence is positioned in a 3′ UTR of an mRNA. In some embodiments, the non-coding sequence is positioned upstream of the polyA tail of the mRNA. In some embodiments, the non-coding sequence is positioned downstream of (e.g., after) the polyA tail of the mRNA. In some embodiments, the non-coding sequence is positioned between the last codon of the ORF of the mRNA and the first “A” of the polyA tail of the mRNA. In some embodiments, a polynucleotide non-coding sequence positioned in a UTR comprises between 1 and 10 nucleotides (e.g., 1, 2, 3, 4, 5, 6, 7, 8, 9, or 10 nucleotides). In some embodiments, UTR comprising a polynucleotide non-coding sequence further comprises one or more (e.g., 1, 2, 3, or more) RNAse cleavage sites, such as RNase H cleavage sites. In some embodiments, each different RNA of a multivalent RNA composition comprises a different (e.g., unique) non-coding sequence. In some embodiments, RNAs of a multivalent RNA composition are detected and/or purified according to the polynucleotide non-coding sequences of the RNAs. In some embodiments, the mRNA non-coding sequences are used to identify the presence of mRNA or determine a relative ratio of different mRNAs in a sample (e.g., a reaction product or a drug product). In some embodiments, the mRNA non-coding sequences are detected using one or more of deep sequencing, PCR, and Sanger sequencing. Exemplary non-coding sequences include: AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; and UGACCA.

In some embodiments the multivalent RNA composition is produced by a method comprising:
(a) combining a linearized first DNA molecule encoding the first mRNA polynucleotide, a linearized second DNA molecule encoding the second mRNA polynucleotide, and a linearized third, fourth, fifth, sixth, seventh, eighth, ninth or tenth DNA molecule encoding the third, fourth, fifth, sixth, seventh, eighth, ninth or tenth mRNA polynucleotide into a single reaction vessel, wherein the first DNA molecule, the second DNA molecule, and the third, fourth, fifth, sixth, seventh, eighth, ninth or tenth DNA molecule are obtained from different sources; and
(b) simultaneously in vitro transcribing the linearized first DNA molecule, the linearized second DNA molecule and the linearized third, fourth, fifth, sixth, seventh, eighth, ninth or tenth DNA molecule to obtain a multivalent RNA composition. The different sources may be bacterial cell cultures which may not be co-cultured. In some embodiments the amounts of the first, second and third, fourth, fifth, sixth, seventh, eighth, ninth or tenth DNA molecules present in the reaction mixture prior to the start of the IVT have been normalized.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle (LNP). Lipid nanoparticles typically comprise ionizable amino lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticles. The vaccines can be made, for example, using mixing processes such as microfluidics and T-junction mixing of two fluid streams, one of which contains the mRNA and the other has the lipid components. In some embodiments, the vaccines are prepared by combining an ionizable amino lipid, a phospholipid (such as DOPE or DSPC), a PEG lipid (such as 1,2-dimyristoyl-OT-glycerol methoxypoly ethylene glycol, also known as PEG-DMG), and a structural lipid (such as cholesterol) in an alcohol (e.g., ethanol). The lipids may be combined to yield desired molar ratios and diluted with water and alcohol (e.g., ethanol) to a final lipid concentration of between about 5.5 mM and about 25 mM, for example.

Vaccines including mRNA and a lipid component may be prepared, for example, by combining a lipid solution with an mRNA solution at lipid component to mRNA wt:wt ratios of between about 5:1 and about 50:1. The lipid solution may be rapidly injected using a microfluidic based system (e.g., NanoAssemblr) at flow rates between about 10 ml/min and about 18 ml/min, for example, into the mRNA solution to produce a suspension (e.g., with a water to alcohol ratio between about 1:1 and about 4:1).

Vaccines can be processed by dialysis to remove the alcohol (e.g., ethanol) and achieve buffer exchange. Formulations may be dialyzed against phosphate buffered saline (PBS), pH 7.4, for example, at volumes greater than that of the primary product (e.g., using Slide-A-Lyzer cassettes (Thermo Fisher Scientific Inc., Rockford, IL)) with a molecular weight cutoff of 10 kD, for example. The forgoing exemplary method induces nanoprecipitation and particle formation. Alternative processes including, but not limited to, T-junction and direct injection, may be used to achieve the same nanoprecipitation.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %.

In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid. In some embodiments, the lipid nanoparticle comprises 40-50 mol % ionizable amino lipid, 5-15 mol % neutral lipid, 20-40 mol % cholesterol, and 0.5-3 mol % PEG-modified lipid. In some embodiments, the lipid nanoparticle comprises 45-50 mol % ionizable amino lipid, 9-13 mol % neutral lipid, 35-45 mol % cholesterol, and 2-3 mol % PEG-modified lipid. In some embodiments, the lipid nanoparticle comprises 48 mol % ionizable amino lipid, 11 mol % neutral lipid, 68.5 mol % cholesterol, and 2.5 mol % PEG-modified lipid.

In some embodiments, an ionizable amino lipid of the disclosure comprises a compound of Formula (I):

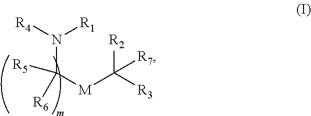

or a salt or isomer thereof, wherein:

R₁ is selected from the group consisting of C₅₋₃₀ alkyl, C₅₋₂₀ alkenyl, -R*YR″, -YR″, and —R″M′R′;

R₂ and R₃ are independently selected from the group consisting of H, C₁₋₄ alkyl, C₂₋₁₄ alkenyl, -R*YR″, -YR″, and —R*OR”, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂),N(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂),OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, -YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR”, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5—to 14-membered heteroaryl having one or more heteroatoms selected from N, O, and S, —OR, —O(CH₂),N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂),OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5—to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R⁸ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′ R′; a13395

R₂ and R₃ are independently selected from the group consisting of H, C₁₋₄ alkyl, C₂₋₁₄ alkenyl, —R*YR″, —YR″, and —R*OR”, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5—to 14-membered heterocycle having one or more heteroatoms selected from N, O, and S, —OR, —O(CH₂),N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂),OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5—to 14-membered heterocycle and (i) R₄ is —(CH₂)ₙQ in which n is 1 or 2, or (ii) R₄ is —(CH₂)·CHQR in which n is 1, or (iii) R₄ is —CHQR, and —CQ(R)₂, then Q is either a 5—to 14-membered heteroaryl or 8—to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR”, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5—to 14-membered heteroaryl having one or more heteroatoms selected from N, O, and S, —OR, —O(CH₂),N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂),OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR”, or R₂ and R₃, together with the atom to which they are attached, form a heterocycle or carbocycle;

R₄ is —(CH₂)ₙQ or —(CH₂)·CHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR”, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, a subset of compounds of Formula (I) includes those of Formula (IA):

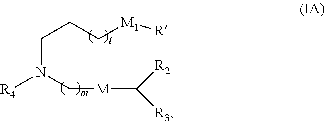

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(=CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₄ alkyl, and C₂₋₁₄ alkenyl.

In some embodiments, a subset of compounds of Formula (I) includes those of Formula (II):

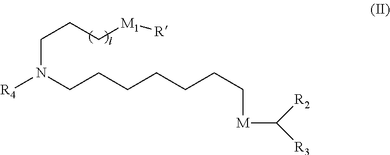

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(=CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₄ alkyl, and C₂₋₁₄ alkenyl.

In some embodiments, a subset of compounds of Formula (I) includes those of Formula (IIa), (IIb), (IIc), or (IIe):

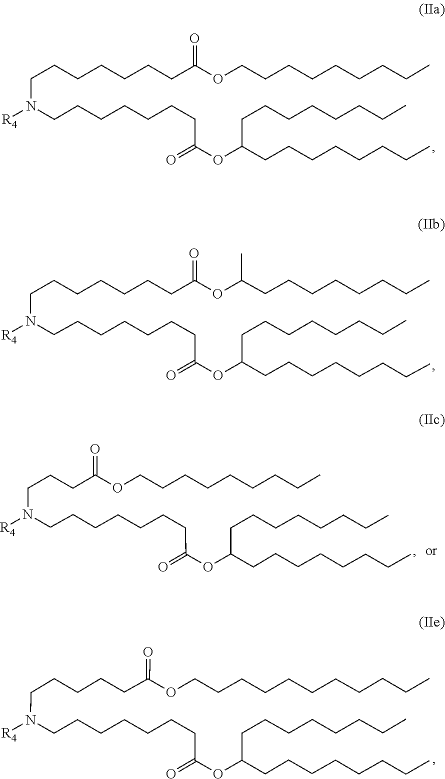

or a salt or isomer thereof, wherein R₄ is as described herein.

In some embodiments, a subset of compounds of Formula (I) includes those of Formula (IId):

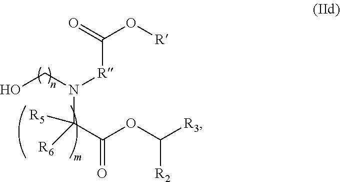

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

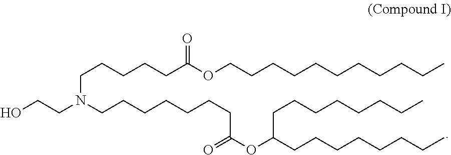 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

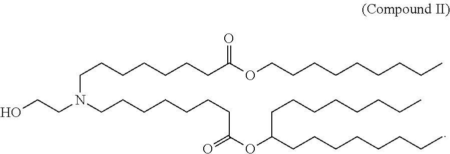 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG (e.g., PEG2000-DMG).

In some embodiments, the lipid nanoparticle comprises 45-55 mole percent (mol %) ionizable amino lipid (e.g., Compound 1). For example, lipid nanoparticle may comprise 45-47, 45-48, 45-49, 45-50, 45-52, 46-48, 46-49, 46-50, 46-52, 46-55, 47-48, 47-49, 47-50, 47-52, 47-55, 48-50, 48-52, 48-55, 49-50, 49-52, 49-55, or 50-55 mol % ionizable amino lipid (e.g., Compound 1). For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol % non-cationic (neutral) lipid (e.g., DSPC). For example, the lipid nanoparticle may comprise 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15,7-8,7-9,7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 13-14, 13-15, or 14-15 mol % non-cationic (neutral) lipid (e.g., DSPC). For example, the lipid nanoparticle may comprise 5, 6, 7, 8, 9, 10, 11, 12, 13, 14, or 15 mol % DSPC.

In some embodiments, the lipid nanoparticle comprises 35-40 mol % sterol (e.g., cholesterol). For example, the lipid nanoparticle may comprise 35-36, 35-37, 35-38, 35-39, 35-40, 36-37, 36-38, 36-39, 36-40, 37-38, 37-39, 37-40, 38-39, 38-40, or 39-40 mol % cholesterol.

For example, the lipid nanoparticle may comprise 35, 35.5, 36, 36.5, 37, 37.5, 38, 38.5, 39, 39.5, or 40 mol % cholesterol.

In some embodiments, the lipid nanoparticle comprises 1-3 mol % DMG-PEG. For example, the lipid nanoparticle may comprise 1-1.5, 1-2, 1-2.5, 1-3, 1.5-2, 1.5-2.5, 1.5-3, 2-2.5, 2-3, or 2.5-3. mol % DMG-PEG. For example, the lipid nanoparticle may comprise 1, 1.5, 2, 2.5, or 3 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG. In some embodiments, the lipid nanoparticle comprises 48 mol % ionizable amino lipid, 11 mol % DSPC, 38.5 mol % cholesterol, and 2.5 mol % PEG2000-DMG.

In some embodiments, an LNP of the disclosure comprises an N:P ratio of from about 2:1 to about 30:1.

In some embodiments, an LNP of the disclosure comprises an N:P ratio of about 6:1.

In some embodiments, an LNP of the disclosure comprises an N:P ratio of about 3:1.

In some embodiments, an LNP of the disclosure comprises a wt/wt ratio of the ionizable amino lipid component to the RNA of from about 10:1 to about 100:1.

In some embodiments, an LNP of the disclosure comprises a wt/wt ratio of the ionizable amino lipid component to the RNA of about 20:1.

In some embodiments, an LNP of the disclosure comprises a wt/wt ratio of the ionizable amino lipid component to the RNA of about 10:1.

In some embodiments, an LNP of the disclosure has a mean diameter from about 50 nm to about 150 nm.

In some embodiments, an LNP of the disclosure has a mean diameter from about 70 nm to about 120 nm.

### Multivalent Vaccines

The compositions, as provided herein, may include RNA or multiple RNAs encoding two or more antigens of the same or different species; that is, the compositions may be multivalent compositions (e.g., vaccines). In some embodiments, the composition includes an RNA or multiple RNAs encoding two or more respiratory virus antigens. In some embodiments, the RNA may encode 1, 2, 3, 4, 5, 6, 7, 8, 9, 10, 11, 12, or more respiratory virus antigens.

In some embodiments, two or more different mRNA encoding antigens may be formulated in the same lipid nanoparticle (e.g., four NA antigens and four HA antigens are formulated in a single lipid nanoparticle or an influenza antigen and a coronavirus antigen are formulated in a single lipid nanoparticle). In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Pharmaceutical Formulations

Provided herein are compositions (e.g., pharmaceutical compositions), methods, kits and reagents for prevention or treatment of respiratory viruses in humans and other mammals, for example. The compositions provided herein can be used as therapeutic or prophylactic agents. They may be used in medicine to prevent and/or treat a respiratory virus infection.

In some embodiments, the respiratory virus vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount is the amount necessary to prevent infection or reduce the severity of a respiratory infection in the subject based on a single dose of the combination vaccine or single dose of the combination vaccine with a booster dose. In some embodiments, an effective amount of the composition containing RNA polynucleotides having at least one chemical modification are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a respiratory virus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A vaccine, disclosed herein, may be administered to a subject to induce an antigen specific immune response, as a combination vaccine (i.e., where both mRNAs encoding antigens are included in the same formulation) or as separate vaccines (i.e, the mRNA encoding the influenza antigen and the mRNA encoding the coronavirus antigen are administered separately). When the vaccine is administered as a separate vaccine, the two mRNAs may be administered to the subject at the same time (i.e., within an hour of one another) or at different times (i.e., separated by more than an hour, 12 hours, 24 hours, 2 days, 7 days, 2 weeks). When the vaccine is administered as a separate vaccine the two mRNAs may be administered to the subject at the same site or a different site (i.e., as an injection in separate arms). In some embodiments the combination vaccine may be the only vaccine comprising a nucleic acid encoding an influenza or coronavirus antigen that a subject receives. Alternatively, the vaccine may be administered in various combinations, as a prime and/or boost dose.

The vaccine may be administered to seropositive or seronegative subjects. For example, a subject may be naive and not have antibodies that react with a virus having an antigen, wherein the antigen is the viral antigen or fragment thereof encoded by the mRNA of the vaccine. Such a subject is said to be seronegative with respect to that vaccine. Alternatively, the subject may have preexisting antibodies to viral antigen encoded by the mRNA of the vaccine because they have previously had an infection with virus carrying the antigen or may have previously been administered a dose of a vaccine (e.g., an mRNA vaccine) that induces antibodies against the antigen. Such a subject is said to be seropositive with respect to that vaccine. In some instances the subject may have been previously exposed to a virus but not to a specific variant or strain of the virus or a specific vaccine associated with that variant or strain. Such a subject is considered to be seronegative with respect to the specific variant or strain.

Thus, the present disclosure provides compositions (e.g., mRNA vaccines) that elicit potent neutralizing antibodies against influenza and coronavirus antigens in a subject. Such a composition can be administered to seropositive or seronegative subjects in some embodiments. A seronegative subject may be naive and not have antibodies that react with the specific virus which the subject is being immunized against. A seropositive subject may have preexisting antibodies to the specific virus because they have previously had an infection with that virus, variant or strain or may have previously been administered a dose of a vaccine (e.g., an mRNA vaccine) that induces antibodies against that virus, variant or strain.

In some embodiments, a composition includes mRNA encoding at least one (e.g., one, two, or more) coronavirus antigens, such as SARS-CoV-2 antigens from different SARS-CoV-2 mutant strains (also referred to herein as variants). In some embodiments, the mRNA vaccine comprises multiple mRNAs encoding SARS-CoV-2 antigens from different variants in a single lipid nanoparticle.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” or “booster vaccine” refers to an extra administration of the prophylactic combination (vaccine) composition. In some embodiments, the booster vaccine comprises at least one mRNA polynucleotide having an ORF encoding the first, second or third respiratory virus antigenic polypeptides. In some embodiments, the booster vaccine comprises at least one mRNA polynucleotide having an ORF encoding each of the first, second and third respiratory virus antigenic polypeptides. In some embodiments, the booster vaccine comprises at least one mRNA polynucleotide having an ORF encoding a variant of the first, second or third respiratory virus antigenic polypeptides.

A booster (or booster vaccine) may be given after an earlier administration of the prophylactic composition. In some embodiments, the combination vaccine is a seasonal booster vaccine (e.g., the combination vaccine is administered annually, such as every autumn or winter).

The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, or 6 months. In exemplary embodiments, the time of administration between the initial administration of the prophylactic composition and the booster may be, but is not limited to, 1 week, 2 weeks, 3 weeks, 1 month, 2 months, 3 months, or 6 months. In one embodiment, the booster vaccine is administered between three weeks and one year after the combination vaccine.

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (they are adjuvant free).

An RNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single—or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of at least one respiratory virus infection in humans and other mammals. Immunizing compositions can be used as therapeutic or prophylactic agents. In some embodiments, immunizing compositions are used to provide prophylactic protection from respiratory virus infections. In some embodiments, immunizing compositions are used to treat respiratory virus infections. In some embodiments, immunizing compositions are used to reduce the severity of a respiratory virus infection in a subjects. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject. In some embodiments, the subject is 60 years of age or older (e.g., 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 years of age or older). In some embodiments, the subject is under 18 years of age (e.g., under 17, 16, 15, 14, 13, 12, 11, 10, 9, 8, 7, 6, 5, 4, 3, 2, or 1 years of age).

In some embodiments, an immunizing composition (e.g., RNA a vaccine) is administered to a subject (e.g., a mammalian subject, such as a human subject) in an effective amount to induce an antigen-specific immune response. The RNA encoding the respiratory virus antigen is expressed and translated in vivo to produce the antigen, which then stimulates an immune response in the subject.

Prophylactic protection from a respiratory virus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer an immunizing composition to an infected individual to achieve a therapeutic response. Dosing may need to be adjusted accordingly.

A method of eliciting an immune response in a subject against a respiratory virus antigen (or multiple antigens) is provided in aspects of the present disclosure. In some embodiments, a method involves administering to the subject an immunizing composition comprising a mRNA having an open reading frame encoding respiratory virus antigen, thereby inducing in the subject an immune response specific to the respiratory virus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the respiratory virus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the respiratory virus or an unvaccinated subject.

A method of eliciting an immune response in a subject against a respiratory virus is provided in other aspects of the disclosure. The method involves administering to the subject an immunizing composition (e.g., an RNA vaccine) comprising a RNA polynucleotide comprising an open reading frame encoding a respiratory virus antigen, thereby inducing in the subject an immune response specific to the respiratory virus, wherein the immune response in the subject is equivalent to an immune response in a subject vaccinated with a traditional vaccine against the respiratory virus at 2 times to 100 times the dosage level relative to the immunizing composition.

In some embodiments, the immune response in the subject is equivalent to an immune response in a subject vaccinated with a traditional vaccine at twice the dosage level relative to an immunizing composition of the present disclosure. In some embodiments, the immune response in the subject is equivalent to an immune response in a subject vaccinated with a traditional vaccine at three times the dosage level relative to an immunizing composition of the present disclosure. In some embodiments, the immune response in the subject is equivalent to an immune response in a subject vaccinated with a traditional vaccine at 4 times, 5 times, 10 times, 50 times, or 100 times the dosage level relative to an immunizing composition of the present disclosure. In some embodiments, the immune response in the subject is equivalent to an immune response in a subject vaccinated with a traditional vaccine at 10 times to 1000 times the dosage level relative to an immunizing composition of the present disclosure. In some embodiments, the immune response in the subject is equivalent to an immune response in a subject vaccinated with a traditional vaccine at 100 times to 1000 times the dosage level relative to an immunizing composition of the present disclosure.

In other embodiments, the immune response is assessed by determining [protein]antibody titer in the subject. In other embodiments, the ability to promote a robust T cell response(s) is measured using art recognized techniques.

Other aspects the disclosure provide methods of eliciting an immune response in a subject against a respiratory virus by administering to the subject an immunizing composition (e.g., an RNA vaccine) comprising an RNA having an open reading frame encoding a respiratory virus antigen, thereby inducing in the subject an immune response specific to the respiratory virus antigen, wherein the immune response in the subject is induced 2 days to 10 weeks earlier relative to an immune response induced in a subject vaccinated with a prophylactically effective dose of a traditional vaccine against the respiratory virus. In some embodiments, the immune response in the subject is induced in a subject vaccinated with a prophylactically effective dose of a traditional vaccine at 2 times to 100 times the dosage level relative to an immunizing composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a respiratory virus by administering to the subject an RNA having an open reading frame encoding a first antigen, wherein the RNA does not include a stabilization element, and wherein an adjuvant is not co-formulated or co-administered with the vaccine.

An immunizing composition (e.g., an RNA vaccine) may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA, as provided herein, may be as low as 25 μg (total mRNA), administered for example as a single dose or as two 12.5 μg doses. A “dose” as used herein, represents the sum total of RNA in the composition (e.g., including all of the NA antigens and/or HA antigens in the formulation). In some embodiments, the effective amount is a total dose of 25 μg-300 μg, 50 μg-300 μg, 100 μg-300 μg, 150 μg-300 μg, 200 μg-300 μg, 250 μg-300 μg, 150 μg-200 μg, 150 μg-250 μg, 150 μg-300 μg, 200 μg-250 μg, or 250 μg-300 μg.

For example, the effective amount may be a total dose of 25 μg, 50 μg, 55 μg, 60 μg, 65 μg, 70 p g, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, or 300 μg. In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 66 μg. In some embodiments, the effective amount is a total dose of 67 μg. In some embodiments, the effective amount is a total dose of 68 μg. In some embodiments, the effective amount is a total dose of 132 μg. In some embodiments, the effective amount is a total dose of 133 μg. In some embodiments, the effective amount is a total dose of 134 μg. In some embodiments, the effective amount is a total dose of 266 μg. In some embodiments, the effective amount is a total dose of 267 μg. In some embodiments, the effective amount is a total dose of 268 μg. In some embodiments, the effective amount is a total dose of 100 μg. In some embodiments, the effective amount is a total dose of 200 μg. In some embodiments, the effective amount is a total dose of 300 μg.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the immunizing compositions (e.g., RNA vaccines), wherein the RNA is formulated in an effective amount to produce an antigen specific immune response in a subject (e.g., production of antibodies specific to respiratory virus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) respiratory virus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-respiratory virus antigen antibody titer produced in a subject administered an immunizing composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an immunizing composition (e.g., RNA vaccine).

In some embodiments, an anti-respiratory virus antigen antibody titer produced in a subject is increased by at least 1 log relative to a control. For example, anti-respiratory virus antigen antibody titer produced in a subject may be increased by at least 1.5, at least 2, at least 2.5, or at least 3 log relative to a control. In some embodiments, the anti-respiratory virus antigen antibody titer produced in the subject is increased by 1, 1.5, 2, 2.5 or 3 log relative to a control.

In some embodiments, the anti-respiratory virus antigen antibody titer produced in the subject is increased by 1-3 log relative to a control. For example, the anti-respiratory virus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-respiratory virus antigen antibody titer produced in a subject is increased at least 2 times relative to a control. For example, the anti-respiratory virus antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-respiratory virus antigen antibody titer produced in the subject is increased 2, 3, 4, 5, 6, 7, 8, 9, or 10 times relative to a control. In some embodiments, the anti-respiratory virus antigen antibody titer produced in a subject is increased 2-10 times relative to a control. For example, the anti-respiratory virus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to a respiratory virus. A geometric mean titer (GMT) is the average antibody titer for a group of subjects calculated by multiplying all values and taking the nth root of the number, where n is the number of subjects with available data.

A control, in some embodiments, is an anti-respiratory virus antigen antibody titer produced in a subject who has not been administered an immunizing composition (e.g., RNA vaccine). In some embodiments, a control is an anti-respiratory virus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an immunizing composition (e.g., RNA vaccine) to be effective is measured in a murine model. For example, an immunizing composition may be administered to a murine model and the murine model assayed for induction of neutralizing antibody titers. Viral challenge studies may also be used to assess the efficacy of a vaccine of the present disclosure. For example, an immunizing composition may be administered to a murine model, the murine model challenged with virus, and the murine model assayed for survival and/or immune response (e.g., neutralizing antibody response, T cell response (e.g., cytokine response)).

A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent a respiratory virus infection or a related condition, while following the standard of care guideline for treating or preventing a respiratory virus infection or a related condition.

In some embodiments, the anti-respiratory virus antigen antibody titer produced in a subject administered an effective amount of an immunizing composition is equivalent to an anti-respiratory virus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( A R U - A R V ) / A R U \times 100 ;$$

$$and Efficacy = ( 1 - R R ) \times 100.$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - O R ) \times 100.$$

In some embodiments, efficacy of the immunizing composition (e.g., RNA vaccine) is at least 60% relative to unvaccinated control subjects. For example, efficacy of the immunizing composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an immunizing composition of the present disclosure is sufficient to provide sterilizing immunity in the subject for at least 1 year. For example, the effective amount of an immunizing composition of the present disclosure is sufficient to provide sterilizing immunity in the subject for at least 2 years, at least 3 years, at least 4 years, or at least 5 years. In some embodiments, the effective amount of an immunizing composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an immunizing composition of the present disclosure is sufficient to produce detectable levels of respiratory virus antigen as measured in serum of the subject at 1-72 hours post administration.

Titer. An antibody titer is a measurement of the amount of antibodies within a subject, for example, antibodies that are specific to a particular antigen (e.g., an anti-respiratory virus antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an immunizing composition of the present disclosure is sufficient to produce a 1,000-10,000 neutralizing antibody titer produced by neutralizing antibody against the respiratory virus antigen as measured in serum of the subject at 1-72 hours post administration. In some embodiments, the effective amount is sufficient to produce a 1,000-5,000 neutralizing antibody titer produced by neutralizing antibody against the respiratory virus antigen as measured in serum of the subject at 1-72 hours post administration. In some embodiments, the effective amount is sufficient to produce a 5,000-10,000 neutralizing antibody titer produced by neutralizing antibody against the respiratory virus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-respiratory virus antigen antibody titer produced in the subject is increased by at least 1 log relative to a control. For example, an anti-respiratory virus antigen antibody titer produced in the subject may be increased by at least 2, 3, 4, 5, 6, 7, 8, 9 or 10 log relative to a control.

In some embodiments, an anti-respiratory virus antigen antibody titer produced in the subject is increased at least 2 times relative to a control. For example, an anti-respiratory virus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### Hemagglutination Inhibition Assay

The hemagglutination inhibition (HAI) test is a classical laboratory procedure for the classification or subtyping of hemagglutinating viruses and further determining the antigenic characteristics of influenza viral isolates provided that the reference antisera used contain antibodies to currently circulating viruses (see, e.g., Pedersen J C Methods Mol Biol. 2014; 1161:11-25). The antisera used are based on antigen preparations derived from either the wildtype strain or a high-growth reassortant made using the wild-type strain or an antigenically equivalent strain.

To perform the assay, a serial dilution of virus is prepared across the rows in a U or V-bottom shaped 96-well microtiter plate. As an example, the most concentrated sample in the first well may be diluted to be 1/5× of the stock, and subsequent wells may be two-fold dilutions (1/10, 1/20, 1/40, etc.). The final well serves as a negative control with no virus. Each row of the plate typically has a different virus and the same pattern of dilutions. After serial dilutions, a standardized concentration of red blood cells (RBCs) is added to each well and mixed gently. The plate is incubated at room temperature. Following the incubation period, the assay can be analyzed to distinguish between agglutinated and non-agglutinated wells. The relative concentration, or titer, of the virus sample is based on the well with the last agglutinated appearance, immediately before a pellet is observed.

Serological methods such as the HAI test are essential for many epidemiological and immunological studies and for evaluation of the antibody response following vaccination. Serological methods are also very useful in situations where identification of the virus is not feasible (e.g. after viral shedding has stopped). The HAI test is used to identify circulating influenza viruses that are antigenically similar to influenza viruses from a previous season's vaccine. As used herein “antigenically similar” refers to a virus having an HAI titer that differs by two dilutions or less.

In some embodiments, the HAI assay is used to measure the effectiveness of a candidate vaccine, such as those provided herein. In some embodiments, the mRNA vaccines have an HAI titer that is 2-, 3-, 4-, 5-, 6-, 7-, 8-, 9-, or 10-fold increased relative to a control (e.g., HAI titer from a subject administered a traditional seasonal flu vaccine, such as FLUBLOK®).

In some embodiments, an HA ELISA assay is performed to examine the HA antibody titers resulting from administration of a candidate vaccine (e.g., IgG antibody titers) (see, e.g., Examples 1, 2, 4, 7, and 8). In some embodiments, the mRNA vaccines have an HA IgG antibody titer that is 1-log, 2-log, 3-log, 4-log, 5-log, 6-log, 7-log, 8-log, 9-log, or 10-log increased relative to a control (e.g., PBS). In some embodiments, the control comprises the HA-reactive IgG antibody titer in a subject prior to administration of the composition (e.g., vaccine).

In some embodiments, a candidate vaccine has an HA IgG antibody titer that is 1-, 2-, 3-, 4-, 5-, 6-, 7-, 8-, 9-, or 10-fold increased relative to a control.

### Neuraminidase Inhibition Assay

The neuraminidase-inhibition (NAI) assay is a laboratory procedure for the identification of the neuraminidase (NA) glycoprotein subtype in influenza viruses or the NA subtype specificity of antibodies to influenza virus (see, e.g., Pedersen J C Methods Mol Biol. 2014; 1161:27-36). A serological procedure for subtyping the NA glycoprotein is critical for the identification and classification of avian influenza (AI) viruses.

There are two basic forms of assay for influenza virus NA based on the use of different substrate molecules, a long-standing assay based on the use of a large substrate such as fetuin (e.g., the enzyme-linked lectin assay (ELLA)) and newer assays which utilize small substrate molecules. The fetuin-based method is used to determine the potency of the viral NA and thus the standardized NA dose for use in the NA inhibition (NAI) assay. Once determined, the standardized dose is added to serial dilutions of test antisera, negative control serum and reference anti-NA serum. Any inhibitory effect of the sera on NA activity can then be determined and the NAI titer calculated. The small substrate based method may be a fluorescence assay that uses the substrate 2-(4-methylumbelliferyl)-a-D-N-acetylneuraminic acid (MUNANA). The substrate is added to serially diluted test antisera and cleavage of the MUNANA substrate by NA releases the fluorescent product methylumbelliferone. The inhibitory effect of the sera on the influenza virus NA is determined based on the concentration of the sera that is required to reduce 50% of the NA activity, given as an IC₅₀ value. The small substrate based method may, alternatively, be a chemiluminescence-based (CL) assay that uses a sialic acid 1,2-dioxetane derivative (NA-Star) substrate or a modified NA-XTD substrate. The CL assays provide an extended-glow chemiluminescent light signal and neuraminidase inhibitor IC₅₀ values are achieved over a range of virus dilutions.

In some embodiments, the mRNA vaccines have an NAI titer that is 2-, 3-, 4-, 5-, 6-, 7-, 8-, 9-, or 10-fold increased relative to a control. The control, in some embodiments, is a traditional seasonal influenza vaccine that only comprises HA antigens (e.g., does not comprise NA antigens). In some embodiments, the control is a NAI titer value for a wild-type NA. In some embodiments, the mRNA vaccine has an NAI titer that is at least 2-fold higher than a control value. In some embodiments, the vaccine's NAI value is at least 50%, 55%, 60%, 65%, 70%, 75%, 80%, 85%, 90%, 95%, 96%, 97%, 98%, or at least 99% of a control (e.g., the NAI value of a wild-type NA).

In some embodiments, an NA ELISA assay is performed to examine the NA antibody titers resulting from administration of a candidate vaccine (e.g., IgG antibody titers) (see, e.g., Examples 1, 2, 4, 7, and 8). In some embodiments, the mRNA vaccines have an NA IgG antibody titer that is 1-log, 2-log, 3-log, 4-log, 5-log, 6-log, 7-log, 8-log, 9-log, or 10-log increased relative to a control (e.g., PBS). In some embodiments, the control comprises the NA-reactive IgG antibody titer in a subject prior to administration of the composition (e.g., vaccine). In some embodiments, a candidate vaccine has an NA IgG antibody titer that is 1-, 2-, 3-, 4-, 5-, 6-, 7-, 8-, 9-, or 10-fold increased relative to a control.

### EXAMPLES

### Example 1. Immunogenicity of Combination Vaccine (Influenza, SARS-CoV-2)

In this example, different combinations of vaccines comprising mRNA encoding influenza and SARS-CoV-2 antigens were tested at high dose (HD) and low dose (LD). For this study, the antigens were formulated separately into different LNPs and mixed before administration. The experiment was carried out as shown below in Table 1. The vaccines included mRNA-1273 (mRNA encoding Spike protein with two proline substitution; SEQ ID NO: 15), mRNA-1010 (four hemagglutinin (HA) antigens combined in ratios (e.g., mass ratios) of 1:1:1:1 to evaluate interference between HAs; SEQ ID NOs: 19, 20, 21, and 22), and mRNA-1020 (four HAs combined with four neuraminidase (NA) antigens in an 8-antigen mixture (i.e., 1:1:1:1:1:1:1:1) to evaluate any interference between HAs in the presence of NAs; SEQ ID NOs: 19, 20, 21, 22, 23, 24, 25 and 26, respectively). All vaccines were tested individually (Groups 2-7) and in combinations of SARS-CoV-2 and influenza vaccine mixtures (Groups 8-11).

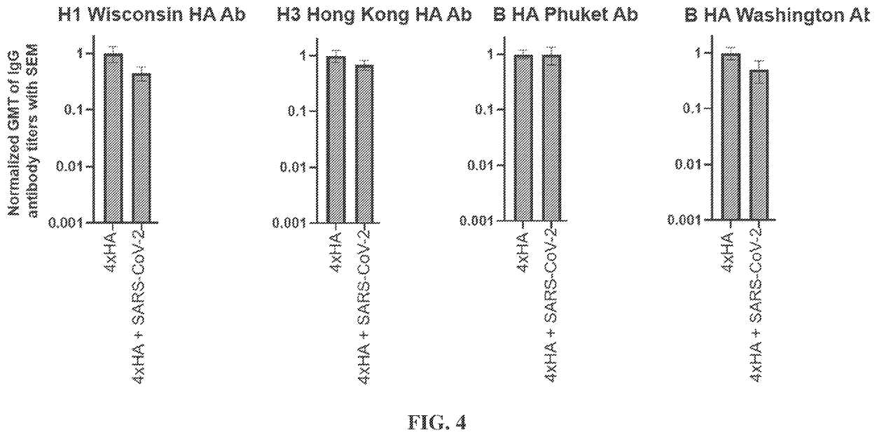

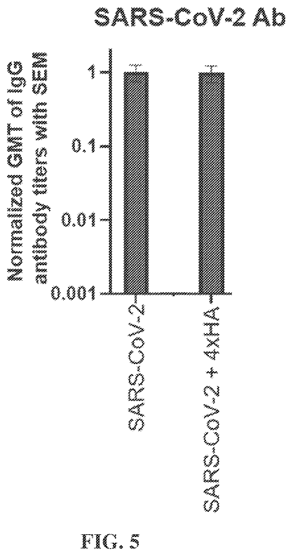

Mice were administered the dose intramuscularly on day 0 and serum samples were collected on day 21. IgG antibody titers were measured by ELISA on individual HA antigens, NA antigens, and SARS-CoV-2 Sp2 recombinant proteins. As shown in FIGS. 1-3, the presence of other antigens in the combination vaccine did not reduce the neutralizing titers against each of the individual antigens in the vaccine (e.g., similar neutralizing titers were observed between the combination vaccine and individual antigen vaccines). FIGS. 4-5 show the results using the normalized geometric mean titer of IgG antibody, and demonstrate that the SARS-CoV-2/influenza (4×HA) at the high dose (Group 8) induced robust antibody responses to all components in the vaccine as compared to individual antigen administration (at the high dose level).

TABLE 1
Study Design

| Group | (n) | Vaccine (prime and boost) | Dose (μg/mouse) |
|---|---|---|---|
| 1 | 4 | PBS | N/A |
| 2 | 8 | SARS-COV-2 (mRNA-1273) | 1.0 |
| 3 | 8 | SARS-COV-2 (mRNA-1273) | 0.2 |
| 4 | 8 | Flu (4 × HA) (mRNA-1010) | 4.0 |
| 5 | 8 | Flu (4 × HA) (mRNA-1010) | 0.8 |
| 6 | 8 | Flu (4 × HA + 4 × NA) (mRNA-1020) | 8.0 |
| 7 | 8 | Flu (4 × HA + 4 × NA) (mRNA-1020) | 1.6 |
| 8 | 8 | SARS-COV-2 + Flu (4 × HA) | 5.0 |
| | | (mRNA1273 + mRNA-1010) | |
| 9 | 8 | SARS-COV-2 + Flu (4 × HA) | 1.0 |
| | | (mRNA1273 + mRNA-1010) | |
| 10 | 8 | SARS-COV-2 + Flu (4 × HA + 4 × NA) | 9.0 |
| | | (mRNA1273 + mRNA-1020) | |
| 11 | 8 | SARS-COV-2 + Flu (4 × HA + 4 × NA) | 1.8 |
| | | (mRNA1273 + mRNA-1020) | |

### Example 2. Immunogenicity of Combination Vaccine (Influenza, SARS-CoV-2) at Different Ratios

Different combinations of vaccines comprising an mRNA encoding influenza and SARS-CoV-2 antigens were tested at a 1:1 and a 2:1 ratio, respectively. The experiment was carried out as shown below in Table 2. The SARS-CoV-2 vaccines comprised mRNA-1273 (mRNA encoding Spike protein with two proline substitutions; SEQ ID NO: 15), mRNA-1283 (mRNA encoding the SARS-CoV-2 Spike protein N-terminal domain, receptor-binding domain, and influenza hemagglutinin transmembrane domain joined by linkers; SEQ ID NO: 17), or mixtures at a 1:1 ratio of mRNA-1273 or mRNA-1283 with mRNA encoding Spike protein of the SARS-CoV-2 B.1.351 (RSA) variant (mRNA-1273.351 and mRNA-1283.351; SEQ ID NOs: 16 and 18, respectively). The influenza vaccines comprised mRNA-1010 (mRNA encoding four HA antigens at a 1:1:1:1 ratio; SEQ ID NOs: 19, 20, 21 and 22). All vaccines were tested individually (Groups 2-7) and in combinations of influenza and SARS-CoV-2 vaccine mixtures at 1:1 (Groups 9, 11, 13, and 15) and 2:1 (Groups 8, 10, 12, and 14) ratios.

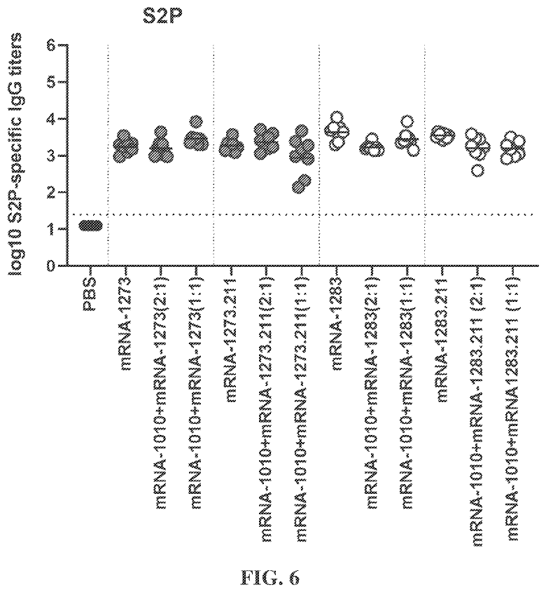

BALB/c mice were administered the dose on day 0 and serum samples were collected on day 21 and day 36. IgG antibody titers were measured by ELISA on individual HA antigens and SARS-CoV-2 SP2 recombinant proteins. Results from day 21 shown in FIGS. 6-11, demonstrate that the presence of other antigens in the combination vaccine did not reduce the neutralizing titers against each of the individual antigens in the vaccine (e.g., similar neutralizing titers were observed between the combination vaccine and individual antigen vaccines). Additionally, similar neutralizing titers against each of the individual antigens in the vaccine were observed between the 1:1 (Groups 9, 11, 13, and 15) and 2:1 (Groups 8, 10, 12, and 14) ratio of influenza:SARS-CoV-2 combination vaccine.

TABLE 2
Study Design

| Group | (n) | Vaccine (prime and boost) | | Dose (μg/mouse) |
|---|---|---|---|---|
| 1 | 4 | PBS | | n/a |
| 2 | 8 | mRNA-1273 | | 1 |
| 3 | 8 | mRNA-1273.211 | | 1 |
| 4 | 8 | mRNA-1283 | | 1 |
| 5 | 8 | mRNA-1283.211 | | 1 |
| 6 | 8 | mRNA-1010 | | 2 |
| 7 | 8 | mRNA-1010 | | 1 |
| 8 | 8 | mRNA-1010 (2 μg) | mRNA-1273 (1 μg) | 3 |
| 9 | 8 | mRNA-1010 (1 μg) | mRNA-1273 (1 μg) | 2 |
| 10 | 8 | mRNA-1010 (2 μg) | mRNA-1273.211 (1 μg) | 3 |
| 11 | 8 | mRNA-1010 (1 μg) | mRNA-1273.211 (1 μg) | 2 |
| 12 | 8 | mRNA-1010 (2 μg) | mRNA-1283 (1 μg) | 3 |
| 13 | 8 | mRNA-1010 (1 μg) | mRNA-1283 (1 μg) | 2 |
| 14 | 8 | mRNA-1010 (2 μg) | mRNA-1283.211 (1 μg) | 3 |
| 15 | 8 | mRNA-1010 (1 μg) | mRNA-1283.211 (1 μg) | 2 |

### Example 3. Immunogenicity of Neuraminidase Antigen Mutations and Ratios of HA/NA Antigens in mRNA Vaccines for Influenza

In this example, the immunogenicity of combinations of neuraminidase (NA) antigen mutations E227D and D151G with various mass ratios of hemagglutinin (HA) to NA antigens is measured as antibody titers in BALB/c mice. The immunogenicity of different vaccines, as outlined in Table 3, are evaluated for antibody titers and dose response, between the 1:1 and 3:1 ratios of HA/NA antigens administered in the mRNA vaccines and compared to the individual antigen. The vaccines include 8 different flu glycoprotein antigens containing either an E227D or D151G neuraminidase antigen mutation tested individually (Groups 2-9 or SEQ ID NOs: 62, 63, 27, 28, 64, 65, 66, 67, respectively), mRNA-1020 (four HA antigens (SEQ ID NOs: 19, 20, 21, 22); and four NA antigens at a 1:1:1:1:1:1:1:1 ratio) combined with a 2021/22 Northern Hemisphere composition containing either a D151G (SEQ ID NOs: 27, 62, 64, 66) or E227D (SEQ ID NOs: 28, 63, 65, 67) neuraminidase antigen mutation (Groups 10 and 11, respectively), or mRNA-1030 (four HA antigens (SEQ ID NOs 19, 20, 21, 22); and four NA antigens at a 3:3:3:3:1:1:1:1 ratio) combined with a 2021/22 Northern Hemisphere composition containing either a D151G (SEQ ID NOs: 27, 62, 64, 66) or E227D (SEQ ID NOs: 28, 63, 65, 67) neuraminidase antigen mutation (Groups 12 and 13, respectively).

BALB/c mice are administered mRNA vaccine or PBS (as a control) and blood samples are taken from the mice on day 21. ELISA assays are used to determine IgG antibody titers to each different influenza glycoprotein antigen.

TABLE 3
Study Design

| | | | Dose Mass | Dose | Read- |
|---|---|---|---|---|---|
| Group | N= | Antigen | (μg/mouse) | Regime | out |
| 1 | 4 | PBS | — | | |
| 2 | 8 | N1 Wisconsin D151G | 1 μg | Prime | ELISA |
| 3 | 8 | N1 Wisconsin E227D | 1 μg | only | |
| 4 | 8 | N2 Cambodia D151G | 1 μg | | |
| 5 | 8 | N2 Cambodia E226D | 1 μg | | |
| 6 | 8 | B Phuket NA D151G | 1 μg | | |
| 7 | 8 | B Phuket NA E227D | 1 μg | | |
| 8 | 8 | B Washington NA D151G | 1 μg | | |
| 9 | 8 | B Washington NA E227D | 1 μg | | |
| 10 | 8 | mRNA-1020-NH21/22 | 8 μg | | |
| | | D151G | | | |
| 11 | 8 | mRNA-1020-NH21/22 | 8 μg | | |
| | | E227D | | | |
| 12 | 8 | mRNA-1030-NH21/22 | 5.33 μg | | |
| | | D151G | | | |
| 13 | 8 | mRNA-1030-NH21/22 | 5.33 μg | | |
| | | E227D | | | |

*NH-Northern Hemisphere

### Example 4. Immunogenicity of Ratios of HA/NA Antigens in mRNA Vaccines for Influenza Viruses Circulating in North and Southern Hemisphere

In this example, the immunogenicity of various mass ratios of HA to NA antigens is measured as antibody titers in BALB/c mice. The immunogenicity of multiple influenza virus HA and NA antigens as mRNA vaccines is evaluated for antibody titers and dose response, between the 1:1 and 3:1 ratios of HA/NA antigens administered in the mRNA vaccines and compared to the individual antigen.

The antigens tested include a mixture of four HA antigens from Northern or Southern Hemisphere strains at a 1:1:1:1 ratio (Groups 2 and 3), or 4 HAs combined with 4 NAs from a Southern Hemisphere strain in an 8-antigen mixture at a 1:1:1:1:1:1:1:1 ratio (Group 5), or a mixture of 4 HAs combined with 4 NAs from a Northern or Southern Hemisphere strain in an 8-antigen mixture at a 3:3:3:3:1:1:1:1 ratio (Groups 6 and 7), or a 5 HA antigen mixture at a 1:1:1:1:1 ratio (Group 8).

BALB/c mice are administered mRNA vaccine or PBS (as a control) on day 1 and day 22 as outlined in Table 4. Blood samples are taken from the mice on day 21 and day 36 and analyzed by ELISA to determine IgG antibody titers to each different influenza glycoprotein antigen.

TABLE 4
Study Design

| | | | Dose Mass | | |
|---|---|---|---|---|---|
| Group | N= | Antigen | (μg/mouse) | Dose Regime | Read-out |
| 1 | 4 | PBS | — | | |
| 2 | 8 | 4 × HA 2021/22 NH strains | 4.8 μg | Two doses, prime on | ELISA |
| | | (Cambodia) | | Day 1 and boost on | |
| 3 | 8 | 4 × HA 2021 SH strains | 4.8 μg | Day 22 | |
| | | (HK) | | | |
| 4 | 30 | 1:1 ratio 4 × HA + 4 × NA | 9.6 μg | | |
| | | 2021/22 NH strains | | | |
| | | (Cambodia) | | | |
| 5 | 8 | 1:1 ratio 4 × HA + 4 × NA | 9.6 μg | | |
| | | 2021 SH strains (HK) | | | |
| 6 | 8 | 3:1 ratio 4 × HA + 4 × NA | 6.4 μg | | |
| | | 2021/22 NH strains | | | |
| | | (Cambodia) | | | |
| 7 | 8 | 3:1 ratio 4 × HA + 4 × NA | 6.4 μg | | |
| | | 2021 SH strains (HK) | | | |
| 8 | 8 | 5 × HA (2021 SH strains + | 6 μg | | |
| | | H3 Cambodia HA) | | | |

*NH-Northern Hemisphere,
SH-Southern Hemisphere

### Example 5. Phase I/II Clinical Trial

This is a Phase 1/2, randomized, stratified, observer-blind study to evaluate the reactogenicity and immunogenicity of a combination vaccine (mRNA-1073) comprising mRNA encoding four different HA antigens and mRNA encoding a SARS-CoV-2 Spike protein having a stabilizing double proline mutation (SEQ ID NO: 33) compared to co-administered mRNA encoding the four different HA antigens (mRNA-1010) and mRNA encoding a SARS-CoV-2 Spike protein having a stabilizing double proline mutation (mRNA-1273; SEQ ID NO: 33) and to the individual vaccines alone in healthy adults 18 to 75 years of age.

On Day 1, each participant will receive 2 injections administered intramuscularly, one in each arm, in the deltoid muscle. The vaccines to be tested include: 1) mRNA-1273, mRNA encoding for the full-length S protein of SARS-CoV-2, modified to introduce S 2P in a prefusion conformation (SEQ ID NO: 33) (50 μg); 2) mRNA-1010, mRNA-1010 encoding for the HA surface glycoproteins of the 4 strains by the WHO for the 2022 NH influenza season cell- or recombinant-based vaccines (50 μg); and 3) mRNA-1073, mRNA encoding for the respective antigens of (1) and (2). This product (mRNA-1073) will also be used for preparing the lower doses of vaccine for Groups 5 and 6. The placebo and the diluent for the vaccine will be 0.9% sodium chloride (normal commercial saline) injection, which meets the criteria of the US Pharmacopeia (USP).

The study will enroll approximately 1050 generally healthy adults 18 to 75 years of age who were previously fully vaccinated for COVID-19 primary series with a locally authorized and approved SARS-CoV-2 vaccine, and their last COVID-19 vaccine (primary series or booster) must be ≥120 days prior (or less per local guidance) to the randomization visit. Participants must not have received a licensed influenza vaccine within ≤180 days of randomization and have no known history of confirmed influenza infection within ≤180 days or SARS-CoV-2 infection within ≤90 days of Screening. The numbers of participants and groups are shown in the below table.

In the Phase 1 portion, randomization will be stratified by age (18 to 49 years old and 50 to 75 years old, balanced across the two age groups within each vaccination group), whereas in the Phase 2 portion, both age groups (18 to 49 years old and 50 to 75 years old, balanced across the 2 age groups within each vaccination group) and receipt of a COVID-19 booster (yes or no) will be stratified at randomization.

Vaccines (mRNA-1073, mRNA-1010, or mRNA-1273) and placebo will be administered as intramuscular (IM) injections, one in each arm, in the deltoid muscle. Safety and/or immunogenicity and/or biomarkers study visits will occur on Days 4, 8, 29, and 181 (end of study).

TABLE 5
Study Arms
Phase 1

| # | Group Name | Sample Size (N = 550) |
|---|---|---|
| 1 | mRNA-1273 (50 μg) + placebo | 50 |
| 2 | mRNA-1010 (50 μg) + placebo | 100 |
| 3 | mRNA-1010 (50 μg) + mRNA-1273 (50 μg) co-administration | 100 |
| 4 | mRNA-1073 (100 μg) + placebo | 100 |
| 5 | mRNA-1073 (50 μg) + placebo | 100 |
| 6 | mRNA-1073 (25 μg) + placebo | 100 |

Phase 2

| # | Group Name | Sample Size (N = 500) |
|---|---|---|
| 1 | mRNA-1010 (dose TBD) + mRNA-1273 (dose TBD) | 250 |
| | co-administration | |
| 2 | mRNA-1073 (dose TBD) + placebo | 250 |

| | |
|---|---|
| Total sample Size for Phases 1 and 2 | 1050 |

### Study Materials

mRNA-1073 is administered as a single dose and aims to elicit protection from influenza and SARS-CoV-2. mRNA-1073 contains mRNA coding for 4 HA antigens of the influenza virus strains recommended for the 2022 NH seasonal vaccines by the WHO and the mRNA for the S protein of SARS-CoV-2 virus formulated in a mixture of 4 lipids common to the Sponsor's mRNA vaccine platform: Compound 1, cholesterol, 1,2-distearoyl-sn-glycero-3-phosphocholine (DSPC), and 1-monomethoxypolyethyleneglycol-2,3-dimyristylglycerol with polyethylene glycol of average molecular weight 2000 (PEG-2000-DMG). mRNA-1073 is based on the antigens encoded for by mRNA-1010 and mRNA-1273 and is intended as a single annual dose for protection from seasonal influenza and SARS-CoV-2. Commercially available 0.9% sodium chloride, USP will be used as appropriate for dose preparation.

mRNA-1010 is administered as a single dose and aims to elicit protection from influenza A and B viruses. mRNA-1010 is a quadrivalent vaccine containing mRNAs encoding for the HAs of the 4 strains recommended by the WHO for 2022 NH cell- or recombinant-based vaccines formulated in a mixture of 4 lipids common to the Sponsor's mRNA vaccine platform: Compound 1, cholesterol, DSPC, and PEG-2000-DMG. Equal amounts of mRNAs encoding for each of the 4 different strains are used for the HA components. mRNA-1010 is administered as a single dose and aims to elicit protection from all seasonal influenza viruses covered by the vaccine.

mRNA-1273 is administered as a single dose and aims to elicit protection from SARS-CoV-2. mRNA-1273 contains mRNA CX-024414 encoding for the S-2P of Wuhan-Hu-1. mRNA-1273 consists of the mRNA formulated in a mixture of 4 lipids common to the Sponsor's mRNA vaccine platform: Compound 1, cholesterol, DSPC, and PEG-2000-DMG.

### Primary Objective

The primary objective is to evaluate the safety and reactogenicity of study vaccines. This will be determined by measuring the frequency and grade of each solicited local and systemic reactogenicity adverse reaction during a 7-day follow-up period post-vaccination, the frequency and severity of any unsolicited AEs during the 28-day follow-up period post vaccination, and the frequency of any SAEs, AESIs, MAAEs, and AEs leading to discontinuation from Day 1 to Day 181/EoS.

### Secondary Objectives

The secondary objectives include the evaluation of the humoral immunogenicity to vaccine-matched strains for influenza and SARS CoV 2 across study vaccine arms at Day 29. This will be accomplished by measuring the GMT and GMFR at Day 29 compared with Day 1 (baseline) by HAI assay for influenza and pseudovirus neutralization assay (PsVNA) (or binding antibody assay) for SARS-CoV-2. For influenza, the percentage of participants with seroconversion, defined as a Day 29 titer>1:40 if baseline is <1:10 or a 4-fold or greater rise if baseline is >1:10 in anti HA antibodies will be measured by HAI assay. For SARS-CoV-2, the percentage of participants with seroresponse, defined as a Day 29 titer>4-fold if baseline is >LLOQ or >4×LLOQ if baseline titer is <LLOQ in nAb titers is measured by PsVNA (or binding antibody assay).

An additional secondary objective is to evaluate the humoral immunogenicity to vaccine-matched strains for influenza and SARS-CoV-2 at all evaluable humoral immunogenicity time points. This will be measured by the GMT and GMFR compared with Day 1 (baseline) by HAI for influenza and PsVNA (or binding antibody assay) for SARS-CoV-2, and by the percentages of participants with seroconversion (influenza) and seroresponse (SARS-CoV-2) as defined above.

### Explorative Objectives

The exploratory objectives include the following: to evaluate the humoral immunogenicity against vaccine mismatched strains (GMT and GMFR (compared to Day 1) to vaccine mismatched strains); to evaluate the humoral immunogenicity against vaccine matched and mismatched strains using alternative methods (GMT and GMFR (compared to Day 1) to vaccine matched and mismatched strains assayed by alternative methods (e.g., microneutralization assay for influenza or ligand-binding assay for SARS CoV 2)); to evaluate cellular immunogenicity in a subset of participants (frequency, magnitude, and phenotype of virus specific T-cell and B-cell responses measured by flow cytometry or other methods, and to perform targeted repertoire analysis of B cells and T cells after vaccination); to further characterize the immune response across study vaccines (frequency, specificities, or other endpoints to be determined for the further characterization of immune responses), and to assess the occurrence of clinical influenza and COVID-19 in study participants and characterize their immune response to infection and viral isolates (frequency of laboratory-confirmed clinical influenza and COVID-19 and assessment of immune responses to infection and viral isolates).

### Immunogenicity Assessments

Blood samples for immunogenicity assessments will be collected per the schedule of events. The following analytes will be measured: serum antibody level as measured by the hemagglutination inhibition assay HAI assay (influenza), serum neutralization antibody level as measured by microneutralization assay (influenza), serum neutralization antibody titers as measured by pseudovirus neutralization assays and/or serum binding antibody titers with ELISAs or multiplex assays (SARS-CoV-2), cellular immunogenicity (in a subset of participants).

### Assessment for Respiratory Viral Infection

During the study, participants might experience symptoms consistent with influenza or SARS-CoV-2 infection. All participants will provide nasopharyngeal (NP) swab samples before the injection on Day 1 for assessment of infection with respiratory pathogens, including influenza viruses and SARS-CoV 2, as influenza or COVID-19 symptoms may confound reactogenicity assessments. Additionally, clinical information will be carefully collected to evaluate the severity of the clinical case.

### Efficacy Assessments:

While the study will not be powered for efficacy assessments, symptoms of infection with respiratory pathogens will be tracked as an exploratory objective in this study.

### Sample Size:

The sample size for this study is not driven by statistical assumptions for formal hypothesis testing. The number of proposed participants is considered sufficient to provide a descriptive summary of the safety and immunogenicity of different study groups. The study will enroll approximately 1050 generally healthy adults 18 to 75 years of age who were previously fully vaccinated for COVID-19 primary series with a locally authorized and approved SARS-CoV-2 vaccine, and their last COVID-19 vaccine must be ≥120 days prior to the randomization visit (or less per local guidance). Participants must not have received a licensed influenza vaccine within ≤180 days of randomization and have not had known history of confirmed influenza infection within ≤180 days or SARS-CoV-2 infection within ≤90 days of screening.

Approximately 550 participants will be enrolled in Phase 1 at a 1:2:2:2:2:2 ratio. Another 500 participants will be enrolled into the Groups 1 and 2 for Phase 2 at a 1:1 ratio.

### Immunogenicity Analyses

The analyses of immunogenicity will be based on the per protocol (PP) set. If the number of participants in the full analysis set (FAS) and PP set differs (defined as the difference divided by the total number of participants in the PP set) by more than 10%, supportive analyses of immunogenicity may be conducted using the FAS.

For the immunogenicity endpoints, the geometric mean of specific antibody titers with corresponding 95% confidence interval (CI) at each time point and the geometric mean fold rise (GMFR) of specific antibody titers with the corresponding 95% CI at each post-baseline time point over pre-injection baseline at Day 1 will be provided by treatment arm, with adjustment for baseline antibody titer and other potential covariates, including age group and primary vaccine type. Descriptive summary statistics, including median, minimum, and maximum, will also be provided.

For summarizations of geometric mean titer, antibody titers reported as below the lower limit of quantification (LLOQ) will be replaced by 0.5×LLOQ. Values that are greater than the upper limit of quantification (ULOQ) will be converted to the ULOQ.

For mRNA-1010, seroconversion rate from baseline will be provided with a 2-sided 95% CI using the Clopper-Pearson method at each post-baseline time point. Rate of seroconversion is defined as the proportion of participants with either a pre-vaccination HAI titer <1:10 and a post-vaccination HAI titer ≥1:40 or a pre-vaccination HAI titer ≥1:10 and a minimum 4-fold rise in post vaccination HAI antibody titer.

For mRNA-1273, seroresponse is defined as either participants with GMFR in neutralizing antibody (nAb) titers binding antibody (bAb) titers of >4 fold at Day 29 compared to Day 1 in those with baseline titer ≥LLOQ, or Day 29 titer ≥4×LLOQ if baseline titer is <LLOQ.

The immunogenicity of mRNA-1073 will follow the same rules as mRNA-1010 and mRNA-1273.

### Interim Analyses

Two interim analyses (IAs) and final analysis will be conducted in the study.

An IA (IA1) will be performed on the data from participants in Phase 1 (550 participants), after they have completed Day 29 visit, and will include the safety and immunogenicity data collected up to Day 29. Either nAb or bAb assay will be used for assessment of immunogenicity on all participants. The dose selection for mRNA-1073 may be supported by the totality of safety and immunogenicity data from the mRNA-1073 groups in IA1 A second IA (A2) will be performed on the data from participants in Phase 2 (500 participants), after they have completed Day 29 visit, and will include the safety data and potentially immunogenicity data collected up to Day 29. The IAs will be performed by a separate team of unblinded programmers and statisticians. The analysis will be presented by vaccination groups. The final analysis of all endpoints will be performed after all participants from Phase 1 and Phase 2 who have completed Day 181/EoS. The final report will include full analyses of all safety and immunogenicity data through Day 181/EoS. For immunogenicity analysis, either nAb or bAb assays will be used on all participants in the Phase 1 study, and potentially on all participants in the Phase 2 study.

SEQUENCE LISTING

| | | |
|---|---|---|
| Name | Sequence | SEQ ID NO: |

mRNA-1273
SEQ ID NO: 33 consists of from 5′ end to 3′ end: 5′UTR SEQ ID NO: 30, mRNA ORF
SEQ ID NO: 1, and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGUUCGUGUUCCUGGUGCUGCUGCCCCUGGUGAGCAGCCAGUG | 1 |
| mRNA | CGUGAACCUGACCACCCGGACCCAGCUGCCACCAGCCUACACCA | |
| (excluding the | ACAGCUUCACCCGGGGCGUCUACUACCCCGACAAGGUGUUCCGG | |
| stop codon) | AGCAGCGUCCUGCACAGCACCCAGGACCUGUUCCUGCCCUUCUU | |
| | CAGCAACGUGACCUGGUUCCACGCCAUCCACGUGAGCGGCACCA | |
| | ACGGCACCAAGCGGUUCGACAACCCCGUGCUGCCCUUCAACGAC | |
| | GGCGUGUACUUCGCCAGCACCGAGAAGAGCAACAUCAUCCGGGG | |
| | CUGGAUCUUCGGCACCACCCUGGACAGCAAGACCCAGAGCCUGC | |
| | UGAUCGUGAAUAACGCCACCAACGUGGUGAUCAAGGUGUGCGAG | |
| | UUCCAGUUCUGCAACGACCCCUUCCUGGGCGUGUACUACCACAA | |
| | GAACAACAAGAGCUGGAUGGAGAGCGAGUUCCGGGUGUACAGC | |
| | AGCGCCAACAACUGCACCUUCGAGUACGUGAGCCAGCCCUUCCU | |
| | GAUGGACCUGGAGGGCAAGCAGGGCAACUUCAAGAACCUGCGGG | |
| | AGUUCGUGUUCAAGAACAUCGACGGCUACUUCAAGAUCUACAGC | |
| | AAGCACACCCCAAUCAACCUGGUGCGGGAUCUGCCCCAGGGCUU | |
| | CUCAGCCCUGGAGCCCCUGGUGGACCUGCCCAUCGGCAUCAACA | |
| | UCACCCGGUUCCAGACCCUGCUGGCCCUGCACCGGAGCUACCUG | |
| | ACCCCAGGCGACAGCAGCAGCGGGUGGACAGCAGGCGCGGCUGC | |
| | UUACUACGUGGGCUACCUGCAGCCCCGGACCUUCCUGCUGAAGU | |
| | ACAACGAGAACGGCACCAUCACCGACGCCGUGGACUGCGCCCUG | |
| | GACCCUCUGAGCGAGACCAAGUGCACCCUGAAGAGCUUCACCGU | |
| | GGAGAAGGGCAUCUACCAGACCAGCAACUUCCGGGUGCAGCCCA | |
| | CCGAGAGCAUCGUGCGGUUCCCCAACAUCACCAACCUGUGCCCC | |
| | UUCGGCGAGGUGUUCAACGCCACCCGGUUCGCCAGCGUGUACGC | |
| | CUGGAACCGGAAGCGGAUCAGCAACUGCGUGGCCGACUACAGCG | |
| | UGCUGUACAACAGCGCCAGCUUCAGCACCUUCAAGUGCUACGGC | |
| | GUGAGCCCCACCAAGCUGAACGACCUGUGCUUCACCAACGUGUA | |
| | CGCCGACAGCUUCGUGAUCCGUGGCGACGAGGUGCGGCAGAUCG | |
| | CACCCGGCCAGACAGGCAAGAUCGCCGACUACAACUACAAGCUG | |
| | CCCGACGACUUCACCGGCUGCGUGAUCGCCUGGAACAGCAACAA | |
| | CCUCGACAGCAAGGUGGGCGGCAACUACAACUACCUGUACCGGC | |
| | UGUUCCGGAAGAGCAACCUGAAGCCCUUCGAGCGGGACAUCAGC | |
| | ACCGAGAUCUACCAAGCCGGCUCCACCCCUUGCAACGGCGUGGA | |
| | GGGCUUCAACUGCUACUUCCCUCUGCAGAGCUACGGCUUCCAGC | |
| | CCACCAACGGCGUGGGCUACCAGCCCUACCGGGUGGUGGUGCUG | |
| | AGCUUCGAGCUGCUGCACGCCCCAGCCACCGUGUGUGGCCCCAA | |
| | GAAGAGCACCAACCUGGUGAAGAACAAGUGCGUGAACUUCAACU | |
| | UCAACGGCCUUACCGGCACCGGCGUGCUGACCGAGAGCAACAAG | |
| | AAAUUCCUGCCCUUUCAGCAGUUCGGCCGGGACAUCGCCGACAC | |
| | CACCGACGCUGUGCGGGAUCCCCAGACCCUGGAGAUCCUGGACA | |
| | UCACCCCUUGCAGCUUCGGCGGCGUGAGCGUGAUCACCCCAGGC | |
| | ACCAACACCAGCAACCAGGUGGCCGUGCUGUACCAGGACGUGAA | |
| | CUGCACCGAGGUGCCCGUGGCCAUCCACGCCGACCAGCUGACAC | |
| | CCACCUGGCGGGUCUACAGCACCGGCAGCAACGUGUUCCAGACC | |
| | CGGGCCGGUUGCCUGAUCGGCGCCGAGCACGUGAACAACAGCUA | |
| | CGAGUGCGACAUCCCCAUCGGCGCCGGCAUCUGUGCCAGCUACC | |
| | AGACCCAGACCAAUUCACCCCGGAGGGCAAGGAGCGUGGCCAGC | |
| | CAGAGCAUCAUCGCCUACACCAUGAGCCUGGGCGCCGAGAACAG | |
| | CGUGGCCUACAGCAACAACAGCAUCGCCAUCCCCACCAACUUCA | |
| | CCAUCAGCGUGACCACCGAGAUUCUGCCCGUGAGCAUGACCAAG | |
| | ACCAGCGUGGACUGCACCAUGUACAUCUGCGGCGACAGCACCGA | |
| | GUGCAGCAACCUGCUGCUGCAGUACGGCAGCUUCUGCACCCAGC | |
| | UGAACCGGGCCCUGACCGGCAUCGCCGUGGAGCAGGACAAGAAC | |
| | ACCCAGGAGGUGUUCGCCCAGGUGAAGCAGAUCUACAAGACCCC | |
| | UCCCAUCAAGGACUUCGGCGGCUUCAACUUCAGCCAGAUCCUGC | |
| | CCGACCCCAGCAAGCCCAGCAAGCGGAGCUUCAUCGAGGACCUG | |
| | CUGUUCAACAAGGUGACCCUAGCCGACGCCGGCUUCAUCAAGCA | |
| | GUACGGCGACUGCCUCGGCGACAUAGCCGCCCGGGACCUGAUCU | |
| | GCGCCCAGAAGUUCAACGGCCUGACCGUGCUGCCUCCCCUGCUG | |
| | ACCGACGAGAUGAUCGCCCAGUACACCAGCGCCCUGUUAGCCGG | |
| | AACCAUCACCAGCGGCUGGACUUUCGGCGCUGGAGCCGCUCUGC | |
| | AGAUCCCCUUCGCCAUGCAGAUGGCCUACCGGUUCAACGGCAUC | |
| | GGCGUGACCCAGAACGUGCUGUACGAGAACCAGAAGCUGAUCGC | |
| | CAACCAGUUCAACAGCGCCAUCGGCAAGAUCCAGGACAGCCUGA | |
| | GCAGCACCGCUAGCGCCCUGGGCAAGCUGCAGGACGUGGUGAAC | |
| | CAGAACGCCCAGGCCCUGAACACCCUGGUGAAGCAGCUGAGCAG | |
| | CAACUUCGGCGCCAUCAGCAGCGUGCUGAACGACAUCCUGAGCC | |
| | GGCUGGACCCUCCCGAGGCCGAGGUGCAGAUCGACCGGCUGAUC | |
| | ACUGGCCGGCUGCAGAGCCUGCAGACCUACGUGACCCAGCAGCU | |
| | GAUCCGGGCCGCCGAGAUUCGGGCCAGCGCCAACCUGGCCGCCA | |
| | CCAAGAUGAGCGAGUGCGUGCUGGGCCAGAGCAAGCGGGUGGAC | |
| | UUCUGCGGCAAGGGCUACCACCUGAUGAGCUUUCCCCAGAGCGC | |
| | ACCCCACGGAGUGGUGUUCCUGCACGUGACCUACGUGCCCGCCC | |
| | AGGAGAAGAACUUCACCACCGCCCCAGCCAUCUGCCACGACGGC | |
| | AAGGCCCACUUUCCCCGGGAGGGCGUGUUCGUGAGCAACGGCAC | |
| | CCACUGGUUCGUGACCCAGCGGAACUUCUACGAGCCCCAGAUCA | |
| | UCACCACCGACAACACCUUCGUGAGCGGCAACUGCGACGUGGUG | |
| | AUCGGCAUCGUGAACAACACCGUGUACGAUCCCCUGCAGCCCGA | |
| | GCUGGACAGCUUCAAGGAGGAGCUGGACAAGUACUUCAAGAAUC | |
| | ACACCAGCCCCGACGUGGACCUGGGCGACAUCAGCGGCAUCAAC | |
| | GCCAGCGUGGUGAACAUCCAGAAGGAGAUCGAUCGGCUGAACGA | |
| | GGUGGCCAAGAACCUGAACGAGAGCCUGAUCGACCUGCAGGAGC | |
| | UGGGCAAGUACGAGCAGUACAUCAAGUGGCCCUGGUACAUCUGG | |
| | CUGGGCUUCAUCGCCGGCCUGAUCGCCAUCGUGAUGGUGACCAU | |
| | CAUGCUGUGCUGCAUGACCAGCUGCUGCAGCUGCCUGAAGGGCU | |
| | GUUGCAGCUGCGGCAGCUGCUGCAAGUUCGACGAGGACGACAGC | |
| | GAGCCCGUGCUGAAGGGCGUGAAGCUGCACUACACC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MFVFLVLLPLVSSQCVNLTTRTQLPPAYTNSFTRGVYYPDKVFRSSVL | 15 |
| amino acid | HSTQDLFLPFFSNVTWFHAIHVSGTNGTKRFDNPVLPFNDGVYFASTE | |
| sequence | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLRE | |
| | FVFKNIDGYFKIYSKHTPINLVRDLPQGFSALEPLVDLPIGINITRFQTL | |
| | LALHRSYLTPGDSSSGWTAGAAAYYVGYLQPRTFLLKYNENGTITDA | |
| | VDCALDPLSETKCTLKSFTVEKGIYQTSNFRVQPTESIVRFPNITNLCPF | |
| | GEVFNATRFASVYAWNRKRISNCVADYSVLYNSASFSTFKCYGVSPT | |
| | KLNDLCFTNVYADSFVIRGDEVRQIAPGQTGKIADYNYKLPDDFTGC | |
| | VIAWNSNNLDSKVGGNYNYLYRLFRKSNLKPFERDISTEIYQAGSTPC | |
| | NGVEGENCYFPLQSYGFQPTNGVGYQPYRVVVLSFELLHAPATVCGP | |
| | KKSTNLVKNKCVNFNFNGLTGTGVLTESNKKFLPFQQFGRDIADTTD | |
| | AVRDPQTLEILDITPCSFGGVSVITPGTNTSNQVAVLYQDVNCTEVPV | |
| | AIHADQLTPTWRVYSTGSNVFQTRAGCLIGAEHVNNSYECDIPIGAGI | |
| | CASYQTQTNSPRRARSVASQSIIAYTMSLGAENSVAYSNNSIAIPTNFT | |
| | ISVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLT | |
| | DEMIAQYTSALLAGTITSGWTFGAGAALQIPFAMQMAYRENGIGVTQ | |
| | NVLYENQKLIANQFNSAIGKIQDSLSSTASALGKLQDVVNQNAQALN | |
| | TLVKQLSSNFGAISSVLNDILSRLDPPEAEVQIDRLITGRLQSLQTYVT | |
| | QQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWF | |
| | VTQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGC | |
| | CSCGSCCKFDEDDSEPVLKGVKLHYT | |
| PolyA Tail | 100 nt | |

mRNA-1273.351
SEQ ID NO: 36 consists of from 5′ end to 3′ end: 5′UTR SEQ ID NO: 30, mRNA ORF SEQ ID
NO: 2, and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGUUCGUGUUCCUGGUGCUGCUGCCCCUGGUGAGCAGCCAGUG | 2 |
| mRNA | CGUGAACUUUACCACCCGGACCCAGCUGCCACCAGCCUACACCA | |
| (excluding the | ACAGCUUCACCCGGGGCGUCUACUACCCCGACAAGGUGUUCCGG | |
| stop codon) | AGCAGCGUCCUGCACAGCACCCAGGACCUGUUCCUGCCCUUCUU | |
| | CAGCAACGUGACCUGGUUCCACGCCAUCCACGUGAGCGGCACCA | |
| | ACGGCACCAAGCGGUUCGCCAACCCCGUGCUGCCCUUCAACGAC | |
| | GGCGUGUACUUCGCCAGCACCGAGAAGAGCAACAUCAUCCGGGG | |
| | CUGGAUCUUCGGCACCACCCUGGACAGCAAGACCCAGAGCCUGC | |
| | UGAUCGUGAAUAACGCCACCAACGUGGUGAUCAAGGUGUGCGAG | |
| | UUCCAGUUCUGCAACGACCCCUUCCUGGGCGUGUACUACCACAA | |
| | GAACAACAAGAGCUGGAUGGAGAGCGAGUUCCGGGUGUACAGC | |
| | AGCGCCAACAACUGCACCUUCGAGUACGUGAGCCAGCCCUUCCU | |
| | GAUGGACCUGGAGGGCAAGCAGGGCAACUUCAAGAACCUGCGGG | |
| | AGUUCGUGUUCAAGAACAUCGACGGCUACUUCAAGAUCUACAGC | |
| | AAGCACACCCCAAUCAACCUGGUGCGGGGCCUGCCCCAGGGCUU | |
| | CUCAGCCCUGGAGCCCCUGGUGGACCUGCCCAUCGGCAUCAACA | |
| | UCACCCGGUUCCAGACCCUGCACAUCAGCUACCUGACCCCAGGC | |
| | GACAGCAGCAGCGGGUGGACAGCAGGCGCGGCUGCUUACUACGU | |
| | GGGCUACCUGCAGCCCCGGACCUUCCUGCUGAAGUACAACGAGA | |
| | ACGGCACCAUCACCGACGCCGUGGACUGCGCCCUGGACCCUCUG | |
| | AGCGAGACCAAGUGCACCCUGAAGAGCUUCACCGUGGAGAAGGG | |
| | CAUCUACCAGACCAGCAACUUCCGGGUGCAGCCCACCGAGAGCA | |
| | UCGUGCGGUUCCCCAACAUCACCAACCUGUGCCCCUUCGGCGAG | |
| | GUGUUCAACGCCACCCGGUUCGCCAGCGUGUACGCCUGGAACCG | |
| | GAAGCGGAUCAGCAACUGCGUGGCCGACUACAGCGUGCUGUACA | |
| | ACAGCGCCAGCUUCAGCACCUUCAAGUGCUACGGCGUGAGCCCC | |
| | ACCAAGCUGAACGACCUGUGCUUCACCAACGUGUACGCCGACAG | |
| | CUUCGUGAUCCGUGGCGACGAGGUGCGGCAGAUCGCACCCGGCC | |
| | AGACAGGCAACAUCGCCGACUACAACUACAAGCUGCCCGACGAC | |
| | UUCACCGGCUGCGUGAUCGCCUGGAACAGCAACAACCUCGACAG | |
| | CAAGGUGGGCGGCAACUACAACUACCUGUACCGGCUGUUCCGGA | |
| | AGAGCAACCUGAAGCCCUUCGAGCGGGACAUCAGCACCGAGAUC | |
| | UACCAAGCCGGCUCCACCCCUUGCAACGGCGUGAAGGGCUUCAA | |
| | CUGCUACUUCCCUCUGCAGAGCUACGGCUUCCAGCCCACCUACG | |
| | GCGUGGGCUACCAGCCCUACCGGGUGGUGGUGCUGAGCUUCGAG | |
| | CUGCUGCACGCCCCAGCCACCGUGUGUGGCCCCAAGAAGAGCAC | |
| | CAACCUGGUGAAGAACAAGUGCGUGAACUUCAACUUCAACGGCC | |
| | UUACCGGCACCGGCGUGCUGACCGAGAGCAACAAGAAAUUCCUG | |
| | CCCUUUCAGCAGUUCGGCCGGGACAUCGCCGACACCACCGACGC | |
| | UGUGCGGGAUCCCCAGACCCUGGAGAUCCUGGACAUCACCCCUU | |
| | GCAGCUUCGGCGGCGUGAGCGUGAUCACCCCAGGCACCAACACC | |
| | AGCAACCAGGUGGCCGUGCUGUACCAGGGCGUGAACUGCACCGA | |
| | GGUGCCCGUGGCCAUCCACGCCGACCAGCUGACACCCACCUGGC | |
| | GGGUCUACAGCACCGGCAGCAACGUGUUCCAGACCCGGGCCGGU | |
| | UGCCUGAUCGGCGCCGAGCACGUGAACAACAGCUACGAGUGCGA | |
| | CAUCCCCAUCGGCGCCGGCAUCUGUGCCAGCUACCAGACCCAGA | |
| | CCAAUUCACCCCGGAGGGCAAGGAGCGUGGCCAGCCAGAGCAUC | |
| | AUCGCCUACACCAUGAGCCUGGGCGUGGAGAACAGCGUGGCCUA | |
| | CAGCAACAACAGCAUCGCCAUCCCCACCAACUUCACCAUCAGCG | |
| | UGACCACCGAGAUUCUGCCCGUGAGCAUGACCAAGACCAGCGUG | |
| | GACUGCACCAUGUACAUCUGCGGCGACAGCACCGAGUGCAGCAA | |
| | CCUGCUGCUGCAGUACGGCAGCUUCUGCACCCAGCUGAACCGGG | |
| | CCCUGACCGGCAUCGCCGUGGAGCAGGACAAGAACACCCAGGAG | |
| | GUGUUCGCCCAGGUGAAGCAGAUCUACAAGACCCCUCCCAUCAA | |
| | GGACUUCGGCGGCUUCAACUUCAGCCAGAUCCUGCCCGACCCCA | |
| | GCAAGCCCAGCAAGCGGAGCUUCAUCGAGGACCUGCUGUUCAAC | |
| | AAGGUGACCCUAGCCGACGCCGGCUUCAUCAAGCAGUACGGCGA | |
| | CUGCCUCGGCGACAUAGCCGCCCGGGACCUGAUCUGCGCCCAGA | |
| | AGUUCAACGGCCUGACCGUGCUGCCUCCCCUGCUGACCGACGAG | |
| | AUGAUCGCCCAGUACACCAGCGCCCUGUUAGCCGGAACCAUCAC | |
| | CAGCGGCUGGACUUUCGGCGCUGGAGCCGCUCUGCAGAUCCCCU | |
| | UCGCCAUGCAGAUGGCCUACCGGUUCAACGGCAUCGGCGUGACC | |
| | CAGAACGUGCUGUACGAGAACCAGAAGCUGAUCGCCAACCAGUU | |
| | CAACAGCGCCAUCGGCAAGAUCCAGGACAGCCUGAGCAGCACCG | |
| | CUAGCGCCCUGGGCAAGCUGCAGGACGUGGUGAACCAGAACGCC | |
| | CAGGCCCUGAACACCCUGGUGAAGCAGCUGAGCAGCAACUUCGG | |
| | CGCCAUCAGCAGCGUGCUGAACGACAUCCUGAGCCGGCUGGACC | |
| | CUCCCGAGGCCGAGGUGCAGAUCGACCGGCUGAUCACUGGCCGG | |
| | CUGCAGAGCCUGCAGACCUACGUGACCCAGCAGCUGAUCCGGGC | |
| | CGCCGAGAUUCGGGCCAGCGCCAACCUGGCCGCCACCAAGAUGA | |
| | GCGAGUGCGUGCUGGGCCAGAGCAAGCGGGUGGACUUCUGCGGC | |
| | AAGGGCUACCACCUGAUGAGCUUUCCCCAGAGCGCACCCCACGG | |
| | AGUGGUGUUCCUGCACGUGACCUACGUGCCCGCCCAGGAGAAGA | |
| | ACUUCACCACCGCCCCAGCCAUCUGCCACGACGGCAAGGCCCACU | |
| | UUCCCCGGGAGGGCGUGUUCGUGAGCAACGGCACCCACUGGUUC | |
| | GUGACCCAGCGGAACUUCUACGAGCCCCAGAUCAUCACCACCGA | |
| | CAACACCUUCGUGAGCGGCAACUGCGACGUGGUGAUCGGCAUCG | |
| | UGAACAACACCGUGUACGAUCCCCUGCAGCCCGAGCUGGACAGC | |
| | UUCAAGGAGGAGCUGGACAAGUACUUCAAGAAUCACACCAGCCC | |
| | CGACGUGGACCUGGGCGACAUCAGCGGCAUCAACGCCAGCGUGG | |
| | UGAACAUCCAGAAGGAGAUCGAUCGGCUGAACGAGGUGGCCAAG | |
| | AACCUGAACGAGAGCCUGAUCGACCUGCAGGAGCUGGGCAAGUA | |
| | CGAGCAGUACAUCAAGUGGCCCUGGUACAUCUGGCUGGGCUUCA | |
| | UCGCCGGCCUGAUCGCCAUCGUGAUGGUGACCAUCAUGCUGUGC | |
| | UGCAUGACCAGCUGCUGCAGCUGCCUGAAGGGCUGUUGCAGCUG | |
| | CGGCAGCUGCUGCAAGUUCGACGAGGACGACAGCGAGCCCGUGC | |
| | UGAAGGGCGUGAAGCUGCACUACACC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MFVFLVLLPLVSSQCVNFTTRTQLPPAYTNSFTRGVYYPDKVFRSSVL | 16 |
| amino acid | HSTQDLFLPFFSNVTWFHAIHVSGTNGTKRFANPVLPFNDGVYFASTE | |
| sequence | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLRE | |
| | FVFKNIDGYFKIYSKHTPINLVRGLPQGFSALEPLVDLPIGINITRFQTL | |
| | HISYLTPGDSSSGWTAGAAAYYVGYLQPRTFLLKYNENGTITDAVDC | |
| | ALDPLSETKCTLKSFTVEKGIYQTSNFRVQPTESIVRFPNITNLCPFGEV | |
| | FNATRFASVYAWNRKRISNCVADYSVLYNSASFSTFKCYGVSPTKLN | |
| | DLCFTNVYADSFVIRGDEVRQIAPGQTGNIADYNYKLPDDFTGCVIA | |
| | WNSNNLDSKVGGNYNYLYRLFRKSNLKPFERDISTEIYQAGSTPCNG | |
| | VKGFNCYFPLQSYGFQPTYGVGYQPYRVVVLSFELLHAPATVCGPKK | |
| | STNLVKNKCVNFNFNGLTGTGVLTESNKKFLPFQQFGRDIADTTDAV | |
| | RDPQTLEILDITPCSFGGVSVITPGTNTSNQVAVLYQGVNCTEVPVAIH | |
| | ADQLTPTWRVYSTGSNVFQTRAGCLIGAEHVNNSYECDIPIGAGICAS | |
| | YQTQTNSPRRARSVASQSIIAYTMSLGVENSVAYSNNSIAIPTNFTISVT | |
| | TEILPVSMTKTSVDCTMYICGDSTECSNLLLQYGSFCTQLNRALTGIA | |
| | VEQDKNTQEVFAQVKQIYKTPPIKDFGGFNFSQILPDPSKPSKRSFIED | |
| | LLFNKVTLADAGFIKQYGDCLGDIAARDLICAQKFNGLTVLPPLLTDE | |
| | MIAQYTSALLAGTITSGWTFGAGAALQIPFAMQMAYRENGIGVTQNV | |
| | LYENQKLIANQFNSAIGKIQDSLSSTASALGKLQDVVNQNAQALNTL | |
| | VKQLSSNFGAISSVLNDILSRLDPPEAEVQIDRLITGRLQSLQTYVTQQ | |
| | LIRAAEIRASANLAATKMSECVLGQSKRVDFCGKGYHLMSFPQSAPH | |
| | GVVFLHVTYVPAQEKNFTTAPAICHDGKAHFPREGVFVSNGTHWFVT | |
| | QRNFYEPQIITTDNTFVSGNCDVVIGIVNNTVYDPLQPELDSFKEELDK | |
| | YFKNHTSPDVDLGDISGINASVVNIQKEIDRLNEVAKNLNESLIDLQEL | |
| | GKYEQYIKWPWYIWLGFIAGLIAIVMVTIMLCCMTSCCSCLKGCCSC | |
| | GSCCKFDEDDSEPVLKGVKLHYT | |
| polyA Tail | 100 nt | |

mRNA-1283
SEQ ID NO: 34 consists of from 5′ end to 3′ end: 5′UTR SEQ ID NO: 30, mRNA ORF SEQ ID
NO: 3 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGUUCGUGUUCCUGGUGCUGCUGCCCCUGGUGAGCAGCCAGUG | 3 |
| mRNA | CGUGAACCUGACCACCCGGACCCAGCUGCCACCAGCCUACACCA | |
| (excluding the | ACAGCUUCACCCGGGGCGUCUACUACCCCGACAAGGUGUUCCGG | |
| stop codon) | AGCAGCGUCCUGCACAGCACCCAGGACCUGUUCCUGCCCUUCUU | |
| | CAGCAACGUGACCUGGUUCCACGCCAUCCACGUGAGCGGCACCA | |
| | ACGGCACCAAGCGGUUCGACAACCCCGUGCUGCCCUUCAACGAC | |
| | GGCGUGUACUUCGCCAGCACCGAGAAGAGCAACAUCAUCCGGGG | |
| | CUGGAUCUUCGGCACCACCCUGGACAGCAAGACCCAGAGCCUGC | |
| | UGAUCGUGAAUAACGCCACCAACGUGGUGAUCAAGGUGUGCGAG | |
| | UUCCAGUUCUGCAACGACCCCUUCCUGGGCGUGUACUACCACAA | |
| | GAACAACAAGAGCUGGAUGGAGAGCGAGUUCCGGGUGUACAGC | |
| | AGCGCCAACAACUGCACCUUCGAGUACGUGAGCCAGCCCUUCCU | |
| | GAUGGACCUGGAGGGCAAGCAGGGCAACUUCAAGAACCUGCGGG | |
| | AGUUCGUGUUCAAGAACAUCGACGGCUACUUCAAGAUCUACAGC | |
| | AAGCACACCCCAAUCAACCUGGUGCGGGAUCUGCCCCAGGGCUU | |
| | CUCAGCCCUGGAGCCCCUGGUGGACCUGCCCAUCGGCAUCAACA | |
| | UCACCCGGUUCCAGACCCUGCUGGCCCUGCACCGGAGCUACCUG | |
| | ACCCCAGGCGACAGCAGCAGCGGGUGGACAGCAGGCGCGGCUGC | |
| | UUACUACGUGGGCUACCUGCAGCCCCGGACCUUCCUGCUGAAGU | |
| | ACAACGAGAACGGCACCAUCACCGACGCCGUGGACGGAGGCGGA | |
| | UCGGGAGGCGGACCCAACAUCACCAACCUGUGCCCCUUCGGCGA | |
| | GGUGUUCAACGCCACCCGGUUCGCCAGCGUGUACGCCUGGAACC | |
| | GGAAGCGGAUCAGCAACUGCGUGGCCGACUACAGCGUGCUGUAC | |
| | AACAGCGCCAGCUUCAGCACCUUCAAGUGCUACGGCGUGAGCCC | |
| | CACCAAGCUGAACGACCUGUGCUUCACCAACGUGUACGCCGACA | |
| | GCUUCGUGAUCCGUGGCGACGAGGUGCGGCAGAUCGCACCCGGC | |
| | CAGACAGGCAAGAUCGCCGACUACAACUACAAGCUGCCCGACGA | |
| | CUUCACCGGCUGCGUGAUCGCCUGGAACAGCAACAACCUCGACA | |
| | GCAAGGUGGGGGCAACUACAACUACCUGUACCGGCUGUUCCGG | |
| | AAGAGCAACCUGAAGCCCUUCGAGCGGGACAUCAGCACCGAGAU | |
| | CUACCAAGCCGGCUCCACCCCUUGCAACGGCGUGGAGGGCUUCA | |
| | ACUGCUACUUCCCUCUGCAGAGCUACGGCUUCCAGCCCACCAAC | |
| | GGCGUGGGCUACCAGCCCUACCGGGUGGUGGUGCUGAGCUUCGA | |
| | GCUGCUGCACGCCCCAGCCACCGUGUGUGGCCCCAAGUCUGGCG | |
| | GAGGCAGCAUCCUGGCCAUCUACAGCACCGUGGCCAGCAGCCUG | |
| | GUGCUGCUGGUGAGCCUGGGCGCCAUCAGCUUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MFVFLVLLPLVSSQCVNLTTRTQLPPAYTNSFTRGVYYPDKVFRSSVL | 17 |
| amino acid | HSTQDLFLPFFSNVTWFHAIHVSGTNGTKRFDNPVLPFNDGVYFASTE | |
| sequence | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLRE | |
| | FVFKNIDGYFKIYSKHTPINLVRDLPQGFSALEPLVDLPIGINITRFQTL | |
| | LALHRSYLTPGDSSSGWTAGAAAYYVGYLQPRTFLLKYNENGTITDA | |
| | VDgggsgggPNITNLCPFGEVFNATRFASVYAWNRKRISNCVADYSVLY | |
| | NSASFSTFKCYGVSPTKLNDLCFTNVYADSFVIRGDEVRQIAPGQTGK | |
| | IADYNYKLPDDFTGCVIAWNSNNLDSKVGGNYNYLYRLFRKSNLKPF | |
| | ERDISTEIYQAGSTPCNGVEGFNCYFPLQSYGFQPTNGVGYQPYRVVV | |
| | LSFELLHAPATVCGPKsgggsilaiystvasslvllvslgaisf | |
| PolyA Tail | 100 nt | |

mRNA-1283.351
SEQ ID NO: 35 consists of from 5′ end to 3′ end: 5′UTR SEQ ID NO: 30, mRNA ORF SEQ ID
NO: 4 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGUUCGUGUUCCUGGUGCUGCUGCCCCUGGUGAGCAGCCAGUG | 4 |
| mRNA | CGUGAACUUUACCACCCGGACCCAGCUGCCACCAGCCUACACCA | |
| (excluding the | ACAGCUUCACCCGGGGCGUCUACUACCCCGACAAGGUGUUCCGG | |
| stop codon) | AGCAGCGUCCUGCACAGCACCCAGGACCUGUUCCUGCCCUUCUU | |
| | CAGCAACGUGACCUGGUUCCACGCCAUCCACGUGAGCGGCACCA | |
| | ACGGCACCAAGCGGUUCGCCAACCCCGUGCUGCCCUUCAACGAC | |
| | GGCGUGUACUUCGCCAGCACCGAGAAGAGCAACAUCAUCCGGGG | |
| | CUGGAUCUUCGGCACCACCCUGGACAGCAAGACCCAGAGCCUGC | |
| | UGAUCGUGAAUAACGCCACCAACGUGGUGAUCAAGGUGUGCGAG | |
| | UUCCAGUUCUGCAACGACCCCUUCCUGGGCGUGUACUACCACAA | |
| | GAACAACAAGAGCUGGAUGGAGAGCGAGUUCCGGGUGUACAGC | |
| | AGCGCCAACAACUGCACCUUCGAGUACGUGAGCCAGCCCUUCCU | |
| | GAUGGACCUGGAGGGCAAGCAGGGCAACUUCAAGAACCUGCGGG | |
| | AGUUCGUGUUCAAGAACAUCGACGGCUACUUCAAGAUCUACAGC | |
| | AAGCACACCCCAAUCAACCUGGUGCGGGGCCUGCCCCAGGGCUU | |
| | CUCAGCCCUGGAGCCCCUGGUGGACCUGCCCAUCGGCAUCAACA | |
| | UCACCCGGUUCCAGACCCUGCACAUCAGCUACCUGACCCCAGGC | |
| | GACAGCAGCAGCGGGUGGACAGCAGGCGCGGCUGCUUACUACGU | |
| | GGGCUACCUGCAGCCCCGGACCUUCCUGCUGAAGUACAACGAGA | |
| | ACGGCACCAUCACCGACGCCGUGGACGGAGGCGGAUCGGGAGGC | |
| | GGACCCAACAUCACCAACCUGUGCCCCUUCGGCGAGGUGUUCAA | |
| | CGCCACCCGGUUCGCCAGCGUGUACGCCUGGAACCGGAAGCGGA | |
| | UCAGCAACUGCGUGGCCGACUACAGCGUGCUGUACAACAGCGCC | |
| | AGCUUCAGCACCUUCAAGUGCUACGGCGUGAGCCCCACCAAGCU | |
| | GAACGACCUGUGCUUCACCAACGUGUACGCCGACAGCUUCGUGA | |
| | UCCGUGGCGACGAGGUGCGGCAGAUCGCACCCGGCCAGACAGGC | |
| | AACAUCGCCGACUACAACUACAAGCUGCCCGACGACUUCACCGG | |
| | CUGCGUGAUCGCCUGGAACAGCAACAACCUCGACAGCAAGGUGG | |
| | GCGGCAACUACAACUACCUGUACCGGCUGUUCCGGAAGAGCAAC | |
| | CUGAAGCCCUUCGAGCGGGACAUCAGCACCGAGAUCUACCAAGC | |
| | CGGCUCCACCCCUUGCAACGGCGUGAAGGGCUUCAACUGCUACU | |
| | UCCCUCUGCAGAGCUACGGCUUCCAGCCCACCUACGGCGUGGGC | |
| | UACCAGCCCUACCGGGUGGUGGUGCUGAGCUUCGAGCUGCUGCA | |
| | CGCCCCAGCCACCGUGUGUGGCCCCAAGUCUGGCGGAGGCAGCA | |
| | UCCUGGCCAUCUACAGCACCGUGGCCAGCAGCCUGGUGCUGCUG | |
| | GUGAGCCUGGGCGCCAUCAGCUUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MFVFLVLLPLVSSQCVNFTTRTQLPPAYTNSFTRGVYYPDKVFRSSVL | 18 |
| amino acid | HSTQDLFLPFFSNVTWFHAIHVSGTNGTKRFANPVLPFNDGVYFASTE | |
| sequence | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLRE | |
| | FVFKNIDGYFKIYSKHTPINLVRGLPQGFSALEPLVDLPIGINITRFQTL | |
| | HISYLTPGDSSSGWTAGAAAYYVGYLQPRTFLLKYNENGTITDAVDG | |
| | GGSGGGPNITNLCPFGEVFNATRFASVYAWNRKRISNCVADYSVLYN | |
| | SASFSTFKCYGVSPTKLNDLCFTNVYADSFVIRGDEVRQIAPGQTGNI | |
| | ADYNYKLPDDFTGCVIAWNSNNLDSKVGGNYNYLYRLFRKSNLKPF | |
| | ERDISTEIYQAGSTPCNGVKGFNCYFPLQSYGFQPTYGVGYQPYRVV | |
| | VLSFELLHAPATVCGPKSGGGSILAIYSTVASSLVLLVSLGAISF | |
| PolyA Tail | 100 nt | |

H3_Hongkong_2019_WT
SEQ ID NO: 37 consists of from 5′ end to 3′ end: 5′UTR SEQ ID NO: 30, mRNA ORF SEQ ID
NO: 5 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAAGACCAUCAUCGCCCUGAGCUACAUCCUGUGCCUGGGCUU | 5 |
| mRNA | CACCCAGAAGAUCCCCGGCAACGAUAACAGCACCGCCACCCUGU | |
| (excluding the | GUCUGGGACACCACGCCGUGCCCAACGGCACCAUCGUGAAGACU | |
| stop codon) | AUCACCAACGACCGGAUCGAGGUGACCAACGCCACCGAGCUGGU | |
| | GCAGAACAGCAGCAUCGGCGAGAUCUGCGACAGCCCUCACCAGA | |
| | UCCUGGACGGCGGCAACUGCACCCUGAUCGACGCACUGCUGGGC | |
| | GACCCUCAGUGCGACGGCUUUCAGAACAAGAAGUGGGACCUGUU | |
| | CGUGGAGAGAUCGCGGGCCUACAGCAACUGCUACCCCUACGACG | |
| | UCCCCGACUACGCAAGCCUGAGAAGCCUCGUGGCCUCAAGCGGC | |
| | ACCCUGGAGUUCAAGAACGAGAGCUUCAACUGGGCCGGCGUGAC | |
| | CCAGAACGGCAAGUCAUUCAGCUGCAUCCGGGGCUCCAGCAGCA | |
| | GCUUCUUCUCACGGCUGAACUGGCUGACCCACCUGAACUACACC | |
| | UACCCCGCCCUGAACGUGACCAUGCCCAACAAGGAGCAGUUCGA | |
| | CAAGCUGUACAUCUGGGGAGUGCACCAUCCCGGCACCGACAAGG | |
| | ACCAGAUUAGCCUGUACGCCCAGUCUAGCGGCCGGAUCACCGUG | |
| | AGCACCAAGCGGAGCCAGCAGGCCGUGAUCCCCAACAUCGGCUC | |
| | UCGGCCCAGAAUCCGGGACAUCCCCAGCCGGAUCAGCAUCUACU | |
| | GGACCAUUGUGAAGCCCGGCGACAUCCUGCUGAUCAACUCCACC | |
| | GGCAACCUGAUCGCCCCUCGGGGCUAUUUCAAGAUCCGGAGCGG | |
| | CAAGAGCAGCAUCAUGCGGAGCGACGCCCCUAUCGGCAAGUGCA | |
| | AGAGCGAGUGCAUCACACCCAACGGAAGCAUCCCCAACGACAAG | |
| | CCCUUCCAGAACGUGAACCGGAUAACCUACGGCGCCUGCCCUAG | |
| | AUACGUGAAGCAGAACACCCUGAAGCUGGCCACCGGCAUGCGGA | |
| | ACGUGCCCGAGAAGCAGACUCGGGGCAUCUUCGGCGCCAUCGCC | |
| | GGCUUCAUCGAGAACGGCUGGGAGGGCAUGGUGGACGGCUGGU | |
| | ACGGCUUCCGGCACCAGAACUCUGAGGGCAGAGGACAGGCCGCA | |
| | GACCUGAAGAGCACCCAGGCCGCCAUCGACCAGAUCAACGGCAA | |
| | GCUGAACCGGCUGAUCGGCAAGACCAACGAGAAGUUCCACCAGA | |
| | UCGAGAAGGAGUUCAGCGAGGUGGAGGGCAGGGUACAGGACCU | |
| | GGAGAAGUACGUGGAGGACACCAAGAUCGACCUGUGGAGCUACA | |
| | ACGCCGAGCUGCUGGUAGCCCUGGAGAACCAGCACACCAUCGAC | |
| | CUGACCGACAGCGAGAUGAACAAGCUGUUCGAGAAGACCAAGAA | |
| | GCAGCUGCGGGAGAACGCCGAGGACAUGGGCAACGGCUGCUUCA | |
| | AGAUCUACCACAAGUGCGACAACGCCUGCAUCGGCAGCAUCCGG | |
| | AACGAGACCUACGACCACAACGUGUACCGGGACGAGGCCCUGAA | |
| | CAACCGGUUCCAGAUCAAGGGCGUGGAGCUGAAGAGCGGCUACA | |
| | AGGACUGGAUCCUGUGGAUCAGCUUCGCCAUCUCCUGCUUCCUG | |
| | CUGUGCGUGGCCCUGCUGGGUUUCAUCAUGUGGGCCUGCCAGAA | |
| | GGGCAACAUCCGGUGCAACAUCUGCAUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MKTIIALSYILCLGFTQKIPGNDNSTATLCLGHHAVPNGTIVKTITNDRI | 19 |
| amino acid | EVTNATELVQNSSIGEICDSPHQILDGGNCTLIDALLGDPQCDGFQNK | |
| sequence | KWDLFVERSRAYSNCYPYDVPDYASLRSLVASSGTLEFKNESFNWAG | |
| | VTQNGKSFSCIRGSSSSFFSRLNWLTHLNYTYPALNVTMPNKEQFDKL | |
| | YIWGVHHPGTDKDQISLYAQSSGRITVSTKRSQQAVIPNIGSRPRIRDIP | |
| | SRISIYWTIVKPGDILLINSTGNLIAPRGYFKIRSGKSSIMRSDAPIGKCK | |
| | SECITPNGSIPNDKPFQNVNRITYGACPRYVKQNTLKLATGMRNVPEK | |
| | QTRGIFGAIAGFIENGWEGMVDGWYGFRHQNSEGRGQAADLKSTQA | |
| | AIDQINGKLNRLIGKTNEKFHQIEKEFSEVEGRVQDLEKYVEDTKIDL | |
| | WSYNAELLVALENQHTIDLTDSEMNKLFEKTKKQLRENAEDMGNGC | |
| | FKIYHKCDNACIGSIRNETYDHNVYRDEALNNRFQIKGVELKSGYKD | |
| | WILWISFAISCFLLCVALLGFIMWACQKGNIRCNICI | |
| PolyA Tail | 100 nt | |

B_HA_Washington_2019_WT
SEQ ID NO: 38 consists of from 5′ end to 3′ end: 5′UTR SEQ ID NO: 30, mRNA ORF SEQ ID
NO: 6 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAAGGCCAUCAUCGUGCUGUUAAUGGUGGUGACCAGCAACGC | 6 |
| mRNA | CGACCGGAUCUGCACCGGCAUCACCUCUAGCAACAGCCCUCACG | |
| (excluding the | UGGUGAAGACCGCCACACAGGGCGAGGUGAACGUGACCGGCGUG | |
| stop codon) | AUUCCCCUGACCACCACCCCUACCAAGAGCCACUUCGCCAACCUG | |
| | AAGGGAACCGAGACCCGGGGCAAGCUGUGUCCCAAGUGCCUGAA | |
| | CUGCACCGACCUGGACGUGGCCCUGGGCAGACCCAAGUGCACCG | |
| | GCAAGAUCCCCAGCGCCCGGGUGUCUAUCCUGCACGAAGUGCGG | |
| | CCCGUGACUAGCGGCUGCUUCCCCAUCAUGCACGACCGGACCAA | |
| | GAUCCGGCAGCUGCCCAACCUGCUGCGGGGCUACGAGCACGUGC | |
| | GGCUGAGCACCCACAACGUGAUCAACGCCGAAGACGCACCCGGG | |
| | AGACCAUACGAGAUCGGCACCAGCGGCUCUUGCCCCAACAUCAC | |
| | CAACGGCAACGGCUUCUUCGCUACCAUGGCCUGGGCCGUGCCAA | |
| | AGAACAAGACUGCCACCAACCCUCUGACCAUCGAGGUGCCCUAC | |
| | AUCUGCACCGAGGGCGAGGACCAGAUCACCGUGUGGGGCUUCCA | |
| | CAGCGACAGCGAGACCCAGAUGGCCAAGCUGUACGGCGACAGCA | |
| | AGCCCCAGAAGUUCACCAGCAGCGCCAACGGCGUGACCACCCAC | |
| | UACGUGAGCCAGAUCGGCGGCUUCCCCAACCAGACCGAGGACGG | |
| | CGGCUUACCCCAGAGCGGCCGGAUCGUGGUGGACUACAUGGUGC | |
| | AGAAGAGCGGCAAGACCGGCACCAUCACCUACCAGCGGGGCAUC | |
| | CUGCUGCCACAGAAGGUGUGGUGCGCCUCAGGGCGGUCAAAGGU | |
| | GAUCAAGGGCAGCCUGCCACUGAUUGGCGAGGCCGACUGCCUGC | |
| | ACGAGAAGUACGGCGGCCUGAACAAGAGCAAGCCCUACUACACC | |
| | GGCGAGCACGCCAAGGCAAUCGGCAACUGCCCCAUCUGGGUGAA | |
| | GACACCCCUGAAGCUGGCCAACGGCACCAAGUACCGGCCACCCG | |
| | CCAAACUGCUGAAGGAGCGGGGCUUCUUCGGCGCCAUUGCCGGC | |
| | UUCCUCGAAGGCGGUUGGGAGGGCAUGAUCGCCGGCUGGCACGG | |
| | CUACACUAGCCACGGCGCACACGGAGUAGCAGUGGCCGCCGACC | |
| | UGAAGAGCACCCAGGAGGCCAUCAACAAGAUCACCAAGAACCUG | |
| | AACAGCCUGAGCGAGCUGGAGGUGAAGAAUCUGCAGCGGCUGUC | |
| | UGGCGCUAUGGACGAGCUGCACAACGAGAUCCUGGAGCUGGACG | |
| | AGAAGGUGGACGACUUACGGGCCGACACCAUCAGCAGCCAGAUC | |
| | GAGCUGGCCGUGCUG8CUGAGCAACGAGGGCAUCAUCAACAGCG | |
| | AGGACGAGCACCUGCUGGCCCUGGAGGGAAGCUGAAGAAGAUGC | |
| | UGGGCCCUUCUGCCGUGGAGAUCGGUAACGGCUGCUUCGAGACC | |
| | AAGCACAAGUGCAACCAGACCUGCCUGGAUCGGAUCGCAGCCGG | |
| | CACCUUUGACGCCGGGGAGUUCAGCCUGCCCACCUUCGACAGCC | |
| | UGAACAUCACCGCCGCCAGCCUGAACGACGACGGCCUGGACAAC | |
| | CACACCAUCCUGCUGUACUACUCUACAGCCGCUAGCAGCCUGGC | |
| | CGUGACCCUGAUGAUCGCCAUCUUCGUGGUGUACAUGGUGAGCC | |
| | GGGACAACGUGAGCUGCAGCAUCUGCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MKAIIVLLMVVTSNADRICTGITSSNSPHVVKTATQGEVNVTGVIPLT | 20 |
| amino acid | TTPTKSHFANLKGTETRGKLCPKCLNCTDLDVALGRPKCTGKIPSARV | |
| sequence | SILHEVRPVTSGCFPIMHDRTKIRQLPNLLRGYEHVRLSTHNVINAEDA | |
| | PGRPYEIGTSGSCPNITNGNGFFATMAWAVPKNKTATNPLTIEVPYICT | |
| | EGEDQITVWGFHSDSETQMAKLYGDSKPQKFTSSANGVTTHYVSQIG | |
| | GFPNQTEDGGLPQSGRIVVDYMVQKSGKTGTITYQRGILLPQKVWCA | |
| | SGRSKVIKGSLPLIGEADCLHEKYGGLNKSKPYYTGEHAKAIGNCPIW | |
| | VKTPLKLANGTKYRPPAKLLKERGFFGAIAGFLEGGWEGMIAGWHG | |
| | YTSHGAHGVAVAADLKSTQEAINKITKNLNSLSELEVKNLQRLSGAM | |
| | DELHNEILELDEKVDDLRADTISSQIELAVLLSNEGIINSEDEHLLALER | |
| | KLKKMLGPSAVEIGNGCFETKHKCNQTCLDRIAAGTFDAGEFSLPTFD | |
| | SLNITAASLNDDGLDNHTILLYYSTAASSLAVTLMIAIFVVYMVSRDN | |
| | VSCSICL | |
| PolyA Tail | 100 nt | |

B_HA_Phuket_2013_WT
SEQ ID NO: 39 consists of from 5′ end to 3′ end: 5′UTR SEQ ID NO: 30, mRNA ORF SEQ ID
NO: 7 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAAGGCCAUCAUCGUGCUACUGAUGGUGGUGACCAGCAACGC | 7 |
| mRNA | CGACCGGAUCUGCACCGGCAUCACCAGCAGCAACAGCCCGCACG | |
| (excluding the | UGGUGAAGACCGCCACCCAAGGCGAGGUGAACGUGACCGGCGUG | |
| stop codon) | AUCCCACUGACCACCACUCCCACCAAGAGCUACUUCGCCAACCU | |
| | GAAGGGCACACGGACUCGGGGCAAGCUGUGCCCCGACUGCCUGA | |
| | ACUGCACCGACCUGGACGUGGCCCUGGGCAGACCCAUGUGCGUG | |
| | GGCACCACCCCUUCUGCCAAGGCCAGCAUCCUGCACGAGGUGAG | |
| | ACCCGUGACCAGCGGGUGCUUCCCCAUCAUGCACGACCGGACCA | |
| | AGAUCCGGCAGCUGCCCAACCUGCUGCGGGGCUACGAGAAGAUC | |
| | CGGCUGAGCACCCAGAACGUGAUCGACGCCGAGAAGGCCCCUGG | |
| | AGGUCCCUACCGGCUGGGCACCAGCGGAAGCUGCCCCAACGCCA | |
| | CCAGCAAGAUCGGCUUCUUCGCCACCAUGGCCUGGGCUGUGCCC | |
| | AAGGACAACUACAAGAACGCCACCAAUCCCCUGACCGUGGAGGU | |
| | GCCCUACAUCUGCACCGAGGGCGAGGACCAGAUCACCGUGUGGG | |
| | GCUUCCACAGCGACAACAAGACCCAGAUGAAGAGCCUGUACGGC | |
| | GACAGCAAUCCCCAGAAGUUCACAAGCAGCGCCAACGGCGUGAC | |
| | CACCCACUACGUGAGCCAGAUCGGCGACUUCCCCGACCAGACCG | |
| | AGGACGGAGGGCUGCCUCAGAGUGGCCGGAUCGUGGUGGACUAC | |
| | AUGAUGCAGAAGCCCGGCAAGACCGGCACCAUCGUGUACCAGCG | |
| | GGGCGUGCUGUUGCCUCAGAAAGUUUGGUGUGCCAGCGGCAGGA | |
| | GCAAGGUGAUCAAGGGCAGCCUGCCCCUGAUCGGCGAGGCAGAC | |
| | UGCCUCCACGAGGAGUACGGCGGCCUGAACAAGAGCAAGCCCUA | |
| | CUACACCGGCAAGCACGCCAAGGCCAUCGGCAACUGCCCCAUCU | |
| | GGGUGAAGACCCCUCUGAAGCUGGCCAACGGCACCAAGUACCGG | |
| | CCACCAGCCAAGCUGCUGAAGGAGCGGGGCUUCUUUGGCGCCAU | |
| | UGCCGGCUUCCUCGAGGGAGGCUGGGAGGGCAUGAUCGCCGGCU | |
| | GGCACGGCUACACAAGCCACGGCGCACACGGAGUGGCUGUGGCU | |
| | GCCGACCUGAAGAGCACCCAGGAGGCCAUCAACAAGAUCACCAA | |
| | GAACCUGAACAGCCUGAGCGAGCUGGAGGUGAAGAACCUGCAGC | |
| | GGCUGUCAGGCGCCAUGGACGAGCUGCACAACGAGAUCCUGGAG | |
| | CUGGACGAGAAGGUGGACGACCUGCGUGCCGACACCAUCAGCAG | |
| | CCAGAUCGAGCUGGCCGUGCUGCUGAGCAACGAGGGCAUCAUCA | |
| | ACAGCGAGGACGAGCACCUGCUGGCCCUGGAGCGGAAACUGAAG | |
| | AAGAUGCUGGGACCCUCUGCCGUGGACAUCGGCAACGGCUGCUU | |
| | CGAGACCAAGCACAAGUGCAACCAGACCUGCCUGGAUCGGAUCG | |
| | CCGCCGGAACCUUCAACGCCGGCGAGUUCAGCCUGCCCACCUUC | |
| | GACAGCCUGAACAUCACCGCCGCCAGCCUGAACGACGACGGCCU | |
| | GGACAACCACACCAUCCUGCUGUACUACAGCACUGCCGCCUCAA | |
| | GCCUGGCCGUGACCCUGAUGCUGGCCAUCUUCAUCGUGUACAUG | |
| | GUGAGCCGGGACAACGUGAGCUGCAGCAUCUGCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MKAIIVLLMVVTSNADRICTGITSSNSPHVVKTATQGEVNVTGVIPLT | 21 |
| amino acid | TTPTKSYFANLKGTRTRGKLCPDCLNCTDLDVALGRPMCVGTTPSAK | |
| sequence | ASILHEVRPVTSGCFPIMHDRTKIRQLPNLLRGYEKIRLSTQNVIDAEK | |
| | APGGPYRLGTSGSCPNATSKIGFFATMAWAVPKDNYKNATNPLTVEV | |
| | PYICTEGEDQITVWGFHSDNKTQMKSLYGDSNPQKFTSSANGVTTHY | |
| | VSQIGDFPDQTEDGGLPQSGRIVVDYMMQKPGKTGTIVYQRGVLLPQ | |
| | KVWCASGRSKVIKGSLPLIGEADCLHEEYGGLNKSKPYYTGKHAKAI | |
| | GNCPIWVKTPLKLANGTKYRPPAKLLKERGFFGAIAGFLEGGWEGMI | |
| | AGWHGYTSHGAHGVAVAADLKSTQEAINKITKNLNSLSELEVKNLQ | |
| | RLSGAMDELHNEILELDEKVDDLRADTISSQIELAVLLSNEGIINSEDE | |
| | HLLALERKLKKMLGPSAVDIGNGCFETKHKCNQTCLDRIAAGTENAG | |
| | EFSLPTFDSLNITAASLNDDGLDNHTILLYYSTAASSLAVTLMLAIFIV | |
| | YMVSRDNVSCSICL | |
| PolyA Tail | 100 nt | |

H1_Wisconsin_2019_WT
SEQ ID NO: 40 consists of from 5′ end to 3′ end: 5′UTR SEQ ID NO: 30, mRNA ORF SEQ ID
NO: 8 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAAGGCCAUCCUGGUCGUGAUGCUGUACACCUUCACCACCGC | 8 |
| mRNA | CAACGCCGACACCCUGUGCAUCGGCUACCACGCCAACAACAGCA | |
| (excluding the | CCGACACCGUGGACACCGUGCUGGAGAAGAACGUGACCGUGACC | |
| stop codon) | CACAGCGUGAACCUGCUGGAGGACAAGCACAACGGCAAGCUGUG | |
| | CAAGCUGAGGGGAGUGGCACCCCUGCACCUGGGCAAGUGCAACA | |
| | UCGCCGGCUGGAUCCUGGGCAACCCCGAGUGCGAGAGCCUGAGC | |
| | ACAGCCCGGAGCUGGAGCUACAUCGUGGAGACCAGCAACAGCGA | |
| | CAACGGCACCUGUUACCCCGGCGACUUCAUCAACUACGAGGAGC | |
| | UGCGGGAGCAGCUGAGCAGCGUGAGCAGCUUCGAGCGGUUCGAG | |
| | AUCUUCCCCAAGACCAGCAGCUGGCCCAACCACGACAGCGACAA | |
| | CGGCGUGACAGCAGCCUGUCCACACGCCGGAGCCAAGAGCUUCU | |
| | ACAAGAACCUGAUCUGGCUGGUGAAGAAGGGCAAGAGCUACCCC | |
| | AAGAUCAACCAGACCUACAUCAACGACAAGGGCAAGGAGGUGCU | |
| | GGUGCUGUGGGGCAUCCACCACCCACCUACCAUCGCCGACCAGC | |
| | AGAGCCUGUACCAGAACGCCGACGCCUACGUGUUCGUGGGCACC | |
| | AGCCGGUACAGCAAGAAGUUCAAGCCAGAGAUCGCCACCCGGCC | |
| | CAAGGUGAGAGACCAGGAGGGCCGGAUGAACUACUACUGGACCC | |
| | UGGUGGAGCCCGGAGACAAGAUUACCUUCGAGGCCACCGGCAAC | |
| | CUGGUGGCCCCUCGGUACGCCUUCACCAUGGAACGGGACGCUGG | |
| | CAGCGGCAUCAUCAUCAGCGACACUCCCGUGCACGACUGCAACA | |
| | CCACCUGCCAGACUCCCGAGGGCGCUAUCAACACCAGCCUGCCC | |
| | UUCCAGAACGUGCACCCCAUCACCAUCGGCAAGUGCCCCAAGUA | |
| | CGUAAAGAGCACCAAAUUGCGGCUGGCCACCGGACUCAGGAACG | |
| | UGCCCAGCAUCCAAAGCCGGGGCCUGUUUGGCGCAAUCGCCGGC | |
| | UUCAUCGAGGGCGGCUGGACUGGCAUGGUGGACGGCUGGUACGG | |
| | CUACCACCACCAGAACGAACAGGGGAGCGGCUACGCAGCUGACC | |
| | UGAAGAGCACCCAGAACGCCAUCGACAAGAUCACCAACAAGGUG | |
| | AACAGCGUGAUCGAGAAGAUGAACACCCAGUUCACCGCCGUGGG | |
| | CAAGGAGUUCAACCACCUGGAGAAGCGGAUCGAGAACCUGAACA | |
| | AGAAGGUGGACGACGGCUUCCUGGACAUCUGGACCUACAACGCC | |
| | GAGCUGCUGGUUCUGCUGGAGAACGAGCGGACCCUGGACUAUCA | |
| | CGACAGCAACGUGAAGAACCUGUACGAGAAGGUGCGGAACCAGC | |
| | UGAAGAACAACGCCAAGGAGAUCGGCAACGGCUGCUUCGAGUUC | |
| | UACCACAAGUGCGACAACACCUGCAUGGAGAGCGUGAAGAACGG | |
| | CACCUACGACUACCCCAAGUACAGCGAGGAGGCCAAGCUGAACC | |
| | GGGAGAAGAUCGACGGCGUGAAGCUGGACAGCACCCGGAUCUAC | |
| | CAGAUCCUGGCCAUCUACAGCACCGUGGCCAGCAGCCUGGUGCU | |
| | GGUGGUGAGCCUGGGCGCCAUCAGCUUCUGGAUGUGCAGCAACG | |
| | GCAGCCUGCAGUGCCGGAUCUGCAUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MKAILVVMLYTFTTANADTLCIGYHANNSTDTVDTVLEKNVTVTHS | 22 |
| amino acid | VNLLEDKHNGKLCKLRGVAPLHLGKCNIAGWILGNPECESLSTARSW | |
| sequence | SYIVETSNSDNGTCYPGDFINYEELREQLSSVSSFERFEIFPKTSSWPNH | |
| | DSDNGVTAACPHAGAKSFYKNLIWLVKKGKSYPKINQTYINDKGKE | |
| | VLVLWGIHHPPTIADQQSLYQNADAYVFVGTSRYSKKFKPEIATRPK | |
| | VRDQEGRMNYYWTLVEPGDKITFEATGNLVAPRYAFTMERDAGSGII | |
| | ISDTPVHDCNTTCQTPEGAINTSLPFQNVHPITIGKCPKYVKSTKLRLA | |
| | TGLRNVPSIQSRGLFGAIAGFIEGGWTGMVDGWYGYHHQNEQGSGY | |
| | AADLKSTQNAIDKITNKVNSVIEKMNTQFTAVGKEFNHLEKRIENLN | |
| | KKVDDGFLDIWTYNAELLVLLENERTLDYHDSNVKNLYEKVRNQLK | |
| | NNAKEIGNGCFEFYHKCDNTCMESVKNGTYDYPKYSEEAKLNREKID | |
| | GVKLDSTRIYQILAIYSTVASSLVLVVSLGAISFWMCSNGSLQCRICI | |
| PolyA Tail | 100 nt | |

N2_Hongkong_2019_WT
SEQ ID NO: 41 consists of from 5′ end to 3′ end: 5′UTR SEQ ID NO: 30, mRNA ORF SEQ ID
NO: 9 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCGAACCAGAAGAUCAUCACCAUCGGCAGCGUGAGCCU | 9 |
| mRNA | GACCAUCAGCACCAUCUGCUUCUUCAUGCAGAUCGCCAUCCUGA | |
| (excluding the | UCACCACCGUGACCCUGCACUUCAAGCAGUACGAGUUCAACAGC | |
| stop codon) | CUGCCCAACAACCAGGUGAUGCUGUGCGAGCCCACCAUCAUCGA | |
| | GCGGAACAUCACCGAGAUCGUGUACCUGACCAACACCACCAUCG | |
| | AGAAGGAGAUCUGCCCCAAGCCCGCCGAGUACCGGAACUGGAGC | |
| | AAGCCCCAGUGCGGCAUCACCGGCUUCGCCCCAUUCAGCAAGGA | |
| | CAACAGCAUCAGACUGAGUGCCGGCGGCGACAUCUGGGUGACCC | |
| | GGGAGCCCUACGUGAGCUGCGACCUGGACAAGUGCUACCAGUUC | |
| | GCCCUGGGACAGGGCACCACCCUGAACAACGUGCACAGCAACAA | |
| | CACUGUGCGGGACCGGACCCCAUACCGGACCCUGCUGAUGAACG | |
| | AGCUGGGCGUGCCCUUCCACCUGGGCACCAAGCAGGUGUGCAUC | |
| | GCCUGGAGCAGCAGCAGCUGCCACGACGGCAAGGCCUGGCUGCA | |
| | CGUGUGCAUUACCGGCGACGACAAGAACGCCACCGCCAGCUUCA | |
| | UCUACAACGGCAGGCUGGUGGACAGCGUGGUGAGCUGGAGCAAC | |
| | GACAUCCUGCGGACCCAGGAGAGCGAGUGCGUGUGCAUCAACGG | |
| | CACCUGCACCGUGGUGAUGACUGACGGCAACGCCACCGGCAAGG | |
| | CCGACACCAAGAUCCUGUUCAUCGAGGAGGGGAAGAUCGUGCAC | |
| | ACCAGCAAGCUGUCUGGCAGCGCCCAGCACGUGGAGGAGUGCAG | |
| | CUGCUACCCUCGGUACCCCGGCGUGAGGUGCGUGUGCCGGGACA | |
| | ACUGGAAGGGCAGCAACCGGCCCAUCAUCGACAUCAACAUCAAG | |
| | GACCACAGCAUAGUGAGCAGCUACGUGUGCAGCGGUCUGGUGGG | |
| | CGACACUCCCCGGAAGAGCGACAGCAGCUCCAGCAGCCACUGCC | |
| | UGAACCCCAACAACGAGGAGGGUGGUCACGGCGUGAAGGGCUGG | |
| | GCCUUCGACGACGGCAACGACGUGUGGAUGGGCCGGACCAUCAA | |
| | CGAGACCAGCAGACUGGGCUACGAGACCUUCAAGGUGGUGGAGG | |
| | GCUGGAGCAAUCCCAAGAGCAAGCUGCAGAUCAACCGGCAGGUG | |
| | AUCGUCGAUCGGGGCGAUCGGAGCGGCUACAGCGGCAUCUUCAG | |
| | CGUGGAGGGCAAGAGCUGCAUCAACCGGUGCUUCUACGUGGAGC | |
| | UGAUCCGGGGCCGGAAGGAGGAGACCGAGGUGCUGUGGACCAGC | |
| | AACAGCAUCGUGGUGUUCUGCGGCACCAGCGGCACCUACGGCAC | |
| | CGGAUCCUGGCCAGACGGCGCCGAUCUGAACCUGAUGCACAUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSVSLTISTICFFMQIAILITTVTLHFKQYEFNSLPNNQVM | 23 |
| amino acid | LCEPTIIERNITEIVYLTNTTIEKEICPKPAEYRNWSKPQCGITGFAPFSK | |
| sequence | DNSIRLSAGGDIWVTREPYVSCDLDKCYQFALGQGTTLNNVHSNNTV | |
| | RDRTPYRTLLMNELGVPFHLGTKQVCIAWSSSSCHDGKAWLHVCITG | |
| | DDKNATASFIYNGRLVDSVVSWSNDILRTQESECVCINGTCTVVMTD | |
| | GNATGKADTKILFIEEGKIVHTSKLSGSAQHVEECSCYPRYPGVRCVC | |
| | RDNWKGSNRPIIDINIKDHSIVSSYVCSGLVGDTPRKSDSSSSSHCLNP | |
| | NNEEGGHGVKGWAFDDGNDVWMGRTINETSRLGYETFKVVEGWSN | |
| | PKSKLQINRQVIVDRGDRSGYSGIFSVEGKSCINRCFYVELIRGRKEET | |
| | EVLWTSNSIVVFCGTSGTYGTGSWPDGADLNLMHI | |
| PolyA Tail | 100 nt | |

B_NA_Washington_2019_WT
SEQ ID NO: 42 consists of from 5′ end to 3′ end: 5′UTR SEQ ID NO: 30, mRNA ORF SEQ ID
NO: 10 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUUCUGACCAG | 10 |
| mRNA | CGGAGGCGUGCUGCUGAGCCUGUACGUGAGCGCCAGCCUGAGCU | |
| (excluding the | ACCUGCUGUACAGCGACAUCCUGCUGAAGUUCAGCCCCACCGAG | |
| stop codon) | AUCACCGCACCCACCAUGCCCCUGGACUGCGCCAACGCCAGCAAC | |
| | GUGCAGGCCGUGAACCGGAGCGCCACAAAGGGCGUGACCCUGCU | |
| | GCUGCCCGAGCCAGAGUGGACAUAUCCUCGGCUGAGCUGCCCUG | |
| | GCAGCACCUUCCAGAAGGCCCUGCUGAUCAGCCCACACCGGUUC | |
| | GGCGAGACCAAGGGCAACAGCGCACCCCUGAUCAUCCGGGAGCC | |
| | CUUCGUGGCCUGUGGCCCCAACGAGUGCAAGCACUUCGCCCUGA | |
| | CACACUACGCUGCUCAGCCCGGUGGCUACUACAACGGCACCCGG | |
| | GGUGAUCGGAACAAGCUGCGGCACCUGAUCAGCGUGAAGCUGGG | |
| | CAAGAUCCCCACCGUGGAGAACAGCAUCUUCCACAUGGCCGCCU | |
| | GGUCAGGAAGCGCCUGCCACGACGGCAAGGAGUGGACCUACAUC | |
| | GGCGUGGACGGCCCUGACAACAACGCCCUGCUGAAGGUGAAGUA | |
| | CGGCGAGGCCUACACCGACACCUACCACAGCUACGCCAACAACA | |
| | UCCUGCGGACCCAGGAGAGCGCCUGCAACUGCAUCGGCGGCAAC | |
| | UGCUACCUGAUGAUCACCGACGGCAGCGCUUCUGGCGUGAGCGA | |
| | GUGCCGGUUCCUGAAGAUCCGGGAGGGCCGGAUCAUCAAGGAGA | |
| | UCUUUCCCACCGGCCGGGUGAAGCACACCGAGGAGUGCACCUGC | |
| | GGCUUCGCCAGCAACAAGACCAUCGAGUGCGCCUGCCGGGACAA | |
| | UCGGUACACCGCCAAGCGGCCCUUCGUGAAGCUGAACGUGGAGA | |
| | CCGACACCGCCGAGAUCCGGCUGAUGUGCACCGACACUUAUCUG | |
| | GACACCCCUCGGCCUAACGACGGCAGCAUCACCGGCCCUUGCGA | |
| | GAGCGACGGCGACAAGGGAAGCGGCGGCAUCAAGGGCGGUUUCG | |
| | UGCACCAGCGGAUGAAGAGCAAGAUCGGCCGGUGGUACAGCCGG | |
| | ACCAUGAGCAAGACCGAGCGGAUGGGCAUGGGCCUGUACGUAAA | |
| | GUACGGAGGGGAUCCCUGGGCUGACAGCGACGCCCUGACCUUCA | |
| | GCGGCGUGAUGGUGAGCAUGAAGGAGCCCGGCUGGUACAGCUUC | |
| | GGCUUCGAGAUCAAGGACAAGAAGUGCGACGUGCCCUGCAUCGG | |
| | CAUCGAGAUGGUGCACGACGGCGGCAAGGAGACCUGGCACUCUG | |
| | CCGCCACUGCCAUCUACUGCCUGAUGGGCAGCGGCCAGCUGCUG | |
| | UGGGACACCGUGACCGGCGUGGACAUGGCCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSPTEITAPTM | 24 |
| amino acid | PLDCANASNVQAVNRSATKGVTLLLPEPEWTYPRLSCPGSTFQKALLI | |
| sequence | SPHRFGETKGNSAPLIIREPFVACGPNECKHFALTHYAAQPGGYYNGT | |
| | RGDRNKLRHLISVKLGKIPTVENSIFHMAAWSGSACHDGKEWTYIGV | |
| | DGPDNNALLKVKYGEAYTDTYHSYANNILRTQESACNCIGGNCYLMI | |
| | TDGSASGVSECRFLKIREGRIIKEIFPTGRVKHTEECTCGFASNKTIECA | |
| | CRDNRYTAKRPFVKLNVETDTAEIRLMCTDTYLDTPRPNDGSITGPCE | |
| | SDGDKGSGGIKGGFVHQRMKSKIGRWYSRTMSKTERMGMGLYVKY | |
| | GGDPWADSDALTFSGVMVSMKEPGWYSFGFEIKDKKCDVPCIGIEM | |
| | VHDGGKETWHSAATAIYCLMGSGQLLWDTVTGVDMAL | |
| PolyA Tail | 100 nt | |

N1_Wisconsin_2019_WT
SEQ ID NO: 43 consists of from 5′ end to 3′ end: 5′UTR SEQ ID NO: 30, mRNA ORF SEQ ID
NO: 11 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCCAACCAGAAGAUCAUCACCAUCGGCAGCAUCUGCAU | 11 |
| mRNA | GACCAUCGGCACCGCCAACCUGAUCCUGCAAAUCGGCAACAUCA | |
| (excluding the | UCAGCAUCUGGGUGAGCCACAGCAUCCAGAUCGGCAACCAGAGC | |
| stop codon) | CAGAUCGAGACCUGCAACAAGAGCGUGAUCACCUACGAGAACAA | |
| | CACCUGGGUGAACCAGACCUUCGUGAACAUCAGCAACACCAACA | |
| | GCGCCGCUCGGCAGUCAGUGGCCAGCGUGAAGCUGGCCGGCAAC | |
| | AGCAGCCUGUGCCCCGUUAGUGGCUGGGCCAUCUACAGCAAGGA | |
| | CAACAGCGUGCGGAUCGGCAGCAAGGGCGACGUGUUCGUGAUCC | |
| | GGGAGCCCUUCAUCAGCUGCAGCCCGCUUGAGUGCCGCACCUUC | |
| | UUCCUGACCCAGGGCGCUCUGCUGAACGACAAGCACAGCAACGG | |
| | CACCAUCAAGGACCGGAGCCCCUAUCGGACCCUGAUGAGCUGCC | |
| | CCAUUGGCGAGGUGCCCAGCCCCUACAACAGCCGGUUCGAGUCU | |
| | GUGGCCUGGAGCGCCUCUGCCUGCCACGACGGCACCAACUGGCU | |
| | GACCAUCGGGAUCAGCGGACCCGAUAGCGGAGCAGUGGCCGUGC | |
| | UGAAGUACAACGGCAUCAUCACCGACACCAUCAAGAGCUGGCGG | |
| | AACAAGAUCCUGCGGACCCAGGAGAGCGAGUGCGCCUGCGUGAA | |
| | CGGCAGCUGCUUCACCAUCAUGACCGACGGCCCUAGCGACGGAC | |
| | AGGCCAGCUACAAGAUCUUCCGGAUCGAGAAGGGCAAGAUCAUC | |
| | AAGAGCGUGGAGAUGAAGGCACCCAACUACCACUACGAGGAGUG | |
| | CAGCUGCUACCCCGACAGCAGCGAGAUCACCUGCGUGUGCCGGG | |
| | ACAACUGGCACGGGAGCAACAGGCCCUGGGUGAGCUUCAACCAG | |
| | AACCUGGAGUACCAGAUGGGCUACAUCUGCAGCGGCGUGUUCGG | |
| | CGACAACCCACGGCCCAACGACAAGACUGGCAGCUGCGGUCCGG | |
| | UGAGCAGCAACGGCGCCAACGGCGUGAAGGGCUUCAGCUUCAAG | |
| | UACGGCAACGGCGUGUGGAUCGGCCGGACCAAGAGCAUCAGCAG | |
| | CCGGAAGGGCUUCGAGAUGAUCUGGGACCCCAACGGCUGGACCG | |
| | GCACCGACAACAAGUUCAGCAAGAAGCAGGACAUCGUGGGCAUC | |
| | AACGAGUGGAGCGGCUACAGCGGCAGCUUCGUGCAGCACCCCGA | |
| | GCUGACUGGCCUGAACUGCAUCCGGCCCUGCUUCUGGGUGGAAC | |
| | UGAUACGGGGACGGCCCGAGGAGAACACCAUCUGGACCAGCGGC | |
| | AGCAGCAUCAGCUUCUGCGGCGUGGACAGCGAUAUCGUGGGCUG | |
| | GAGCUGGCCAGACGGAGCCGAGCUGCCCUUCACCAUCGACAAG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSICMTIGTANLILQIGNIISIWVSHSIQIGNQSQIETCNKS | 25 |
| amino acid | VITYENNTWVNQTFVNISNTNSAARQSVASVKLAGNSSLCPVSGWAI | |
| sequence | YSKDNSVRIGSKGDVFVIREPFISCSPLECRTFFLTQGALLNDKHSNGTI | |
| | KDRSPYRTLMSCPIGEVPSPYNSRFESVAWSASACHDGTNWLTIGISG | |
| | PDSGAVAVLKYNGIITDTIKSWRNKILRTQESECACVNGSCFTIMTDG | |
| | PSDGQASYKIFRIEKGKIIKSVEMKAPNYHYEECSCYPDSSEITCVCRD | |
| | NWHGSNRPWVSFNQNLEYQMGYICSGVFGDNPRPNDKTGSCGPVSS | |
| | NGANGVKGFSFKYGNGVWIGRTKSISSRKGFEMIWDPNGWTGTDNK | |
| | FSKKQDIVGINEWSGYSGSFVQHPELTGLNCIRPCFWVELIRGRPEENT | |
| | IWTSGSSISFCGVDSDIVGWSWPDGAELPFTIDK | |
| PolyA Tail | 100 nt | |

B_NA_Phuket_2013_WT
SEQ ID NO: 44 consists of from 5′ end to 3′ end: 5′UTR SEQ ID NO: 30, mRNA ORF SEQ ID
NO: 12 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUCCUGACCAG | 12 |
| mRNA | CGGAGGCGUGCUGCUGAGCCUGUACGUCAGCGCCAGCCUGAGCU | |
| (excluding the | ACCUGCUGUACAGCGACAUCCUGCUGAAGUUCAGCCGGACCGAG | |
| stop codon) | GUGACCGCUCCCAUCAUGCCCCUGGACUGCGCCAACGCCAGCAA | |
| | CGUGCAGGCCGUGAAUCGGAGCGCCACCAAGGGCGUGACUCCCC | |
| | UGCUGCCCGAGCCUGAGUGGACUUAUCCUCGGCUGAGCUGCCCA | |
| | GGCAGCACCUUCCAGAAGGCCCUGCUGAUCAGCCCACACCGGUU | |
| | CGGCGAGACCAAGGGCAACAGCGCUCCCCUGAUCAUCCGGGAGC | |
| | CCUUCAUCGCCUGCGGCCCCAAGGAGUGCAAGCACUUCGCCCUG | |
| | ACCCACUACGCUGCCCAACCCGGAGGCUACUACAACGGCACCAG | |
| | AGAGGACCGGAACAAGCUGCGGCACCUGAUCAGCGUGAAGCUGG | |
| | GCAAGAUCCCCACCGUGGAGAACAGCAUCUUCCACAUGGCUGCU | |
| | UGGUCUGGAAGUGCUUGUCACGACGGCCGGGAGUGGACCUACAU | |
| | CGGCGUGGACGGCCCAGACAGCAACGCCCUGCUGAAGAUCAAGU | |
| | ACGGCGAGGCCUACACCGACACCUACCACAGCUACGCCAAGAAC | |
| | AUCCUGCGGACCCAGGAGAGCGCCUGCAACUGCAUCGGCGGCGA | |
| | CUGCUACCUGAUGAUCACCGACGGCCCAGCAUCUGGCAUCAGCG | |
| | AGUGCCGGUUCCUGAAGAUCCGGGAGGGCCGGAUCAUCAAGGAG | |
| | AUCUUCCCCACCGGGAGAGUGAAGCACACCGAGGAGUGCACCUG | |
| | CGGCUUCGCCAGCAACAAGACCAUCGAGUGCGCCUGCCGGGACA | |
| | ACAGCUACACCGCCAAGCGGCCCUUCGUGAAGCUGAACGUGGAG | |
| | ACCGACACCGCCGAGAUCCGGCUGAUGUGCACCAAGACCUACCU | |
| | GGACACCCCUCGGCCCAACGACGGAAGCAUCACCGGACCCUGCG | |
| | AGAGCGACGGGGACGAAGGAAGCGGCGGAAUCAAGGGCGGCUUC | |
| | GUGCACCAGCGGAUGGCCAGCAAGAUCGGCCGGUGGUACAGCCG | |
| | GACCAUGAGCAAGACCAAGCGGAUGGGCAUGGGCCUGUACGUGA | |
| | AGUACGACGGCGACCCCUGGACAGACAGCGAAGCCCUGGCCCUG | |
| | UCUGGCGUGAUGGUGAGCAUGGAGGAGCCCGGCUGGUACAGCUU | |
| | CGGCUUCGAGAUCAAGGACAAGAAGUGCGACGUGCCCUGCAUCG | |
| | GCAUCGAGAUGGUGCACGACGGCGGCAAGACCACCUGGCAUAGC | |
| | GCCGCAACCGCGAUCUACUGCCUGAUGGGCAGCGGCCAGCUGCU | |
| | GUGGGACACCGUGACCGGCGUGAACAUGACCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSRTEVTAPIM | 26 |
| amino acid | PLDCANASNVQAVNRSATKGVTPLLPEPEWTYPRLSCPGSTFQKALLI | |
| sequence | SPHRFGETKGNSAPLIIREPFIACGPKECKHEALTHYAAQPGGYYNGT | |
| | REDRNKLRHLISVKLGKIPTVENSIFHMAAWSGSACHDGREWTYIGV | |
| | DGPDSNALLKIKYGEAYTDTYHSYAKNILRTQESACNCIGGDCYLMIT | |
| | DGPASGISECRFLKIREGRIIKEIFPTGRVKHTEECTCGFASNKTIECAC | |
| | RDNSYTAKRPFVKLNVETDTAEIRLMCTKTYLDTPRPNDGSITGPCES | |
| | DGDEGSGGIKGGFVHQRMASKIGRWYSRTMSKTKRMGMGLYVKYD | |
| | GDPWTDSEALALSGVMVSMEEPGWYSFGFEIKDKKCDVPCIGIEMVH | |
| | DGGKTTWHSAATAIYCLMGSGQLLWDTVTGVNMTL | |
| PolyA Tail | 100 nt | |

N2_A_Cambodia_2020_D151G
SEQ ID NO: 45 consists of from 5′ end to 3′ end: 5′UTR SEQ ID NO: 30, mRNA ORF SEQ ID
NO: 13 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCGAACCAGAAGAUCAUCACCAUCGGCAGCGUGAGCCU | 13 |
| mRNA | GACCAUCAGCACCAUCUGCUUCUUCAUGCAGAUCGCCAUCCUGA | |
| (excluding the | UCACCACCGUGACCCUGCACUUCAAGCAGUACGAGUUCAACAGC | |
| stop codon) | CCUCCCAACAACCAGGUGAUGCUGUGCGAGCCCACCAUCAUCGA | |
| | GCGGAACAUGACCGAGAUCGUGUACCUGACCAACACCACCAUCG | |
| | AGAAGGAGAUCUGCCCCAAGCCCGCCGAGUACCGGAACUGGAGC | |
| | AAGCCCCAGUGCGGCAUCACCGGCUUCGCCCCAUUCAGCAAGGA | |
| | CAACAGCAUCAGACUGAGUGCCGGCGGCGACAUCUGGGUGACCC | |
| | GGGAGCCCUACGUGAGCUGCGACCUGGACAAGUGCUACCAGUUC | |
| | GCCCUGGGACAGGGCACCACCCUGAACAACGUGCACAGCAACAA | |
| | CACUGUGCGGGGCCGGACCCCAUACCGGACCCUGCUGAUGAACG | |
| | AGCUGGGCGUGCCCUUCCACCUGGGCACCAAGCAGGUGUGCAUC | |
| | GCCUGGAGCAGCAGCAGCUGCCACGACGGCAAGGCCUGGCUGCA | |
| | CGUGUGCAUUACCGGCGACGACAAGAACGCCACCGCCAGCUUCA | |
| | UCUACAACGGCAGGCUGGUGGACAGCGUGGUGAGCUGGAGCAAC | |
| | GACAUCCUGCGGACCCAGGAGAGCGAGUGCGUGUGCAUCAACGG | |
| | CACCUGCACCGUGGUGAUGACUGACGGCAACGCCACCGGCAAGG | |
| | CCGACACCAAGAUCCUGUUCAUCGAGGAGGGGAAGAUCGUGCAC | |
| | ACCAGCAAGCUGUCUGGCAGCGCCCAGCACGUGGAGGAGUGCAG | |
| | CUGCUACCCUCGGUACCCCGGCGUGAGGUGCGUGUGCCGGGACA | |
| | ACUGGAAGGGCAGCAACCGGCCCAUCAUCGACAUCAACAUCAAG | |
| | GACCACAGCAUAGUGAGCAGAUACGUGUGCAGCGGUCUGGUGGG | |
| | CGACACUCCCCGGAAGAGCGACAGCAGCUCCAGCAGCCACUGCC | |
| | UGAACCCCAACAACGAGAAGGGUGACCACGGCGUGAAGGGCUGG | |
| | GCCUUCGACGACGGCAACGACGUGUGGAUGGGCCGGACCAUCAA | |
| | CGAGACCAGCAGACUGGGCUACGAGACCUUCAAGGUGGUGGAGG | |
| | GCUGGAGCAAUCCCAAGAGCAAGCUGCAGAUCAACCGGCAGGUG | |
| | AUCGUCGAUCGGGGCGAUCGGAGCGGCUACAGCGGCAUCUUCAG | |
| | CGUGGAGGGCAAGAGCUGCAUCAACCGGUGCUUCUACGUGGAGC | |
| | UGAUCCGGGGCCGGAAGGAGGAGACCGAGGUGCUGUGGACCAGC | |
| | AACAGCAUCGUGGUGUUCUGCGGCACCAGCGGCACCUACGGCAC | |
| | CGGAUCCUGGCCAGACGGCGCCAACCUGAGCCUGAUGCACAUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSVSLTISTICFFMQIAILITTVTLHFKQYEFNSPPNNQVM | 27 |
| amino acid | LCEPTIIERNMTEIVYLTNTTIEKEICPKPAEYRNWSKPQCGITGFAPFS | |
| sequence | KDNSIRLSAGGDIWVTREPYVSCDLDKCYQFALGQGTTLNNVHSNNT | |
| | VRGRTPYRTLLMNELGVPFHLGTKQVCIAWSSSSCHDGKAWLHVCIT | |
| | GDDKNATASFIYNGRLVDSVVSWSNDILRTQESECVCINGTCTVVMT | |
| | DGNATGKADTKILFIEEGKIVHTSKLSGSAQHVEECSCYPRYPGVRCV | |
| | CRDNWKGSNRPIIDINIKDHSIVSRYVCSGLVGDTPRKSDSSSSSHCLN | |
| | PNNEKGDHGVKGWAFDDGNDVWMGRTINETSRLGYETFKVVEGWS | |
| | NPKSKLQINRQVIVDRGDRSGYSGIFSVEGKSCINRCFYVELIRGRKEE | |
| | TEVLWTSNSIVVFCGTSGTYGTGSWPDGANLSLMHI | |
| PolyA Tail | 100 nt | |

N2_A_Cambodia_2020_E227D
SEQ ID NO: 46 consists of from 5′ end to 3′ end: 5′UTR SEQ ID NO: 30, mRNA ORF SEQ ID
NO: 14 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCGAACCAGAAGAUCAUCACCAUCGGCAGCGUGAGCCU | 14 |
| mRNA | GACCAUCAGCACCAUCUGCUUCUUCAUGCAGAUCGCCAUCCUGA | |
| (excluding the | UCACCACCGUGACCCUGCACUUCAAGCAGUACGAGUUCAACAGC | |
| stop codon) | CCUCCCAACAACCAGGUGAUGCUGUGCGAGCCCACCAUCAUCGA | |
| | GCGGAACAUGACCGAGAUCGUGUACCUGACCAACACCACCAUCG | |
| | AGAAGGAGAUCUGCCCCAAGCCCGCCGAGUACCGGAACUGGAGC | |
| | AAGCCCCAGUGCGGCAUCACCGGCUUCGCCCCAUUCAGCAAGGA | |
| | CAACAGCAUCAGACUGAGUGCCGGCGGCGACAUCUGGGUGACCC | |
| | GGGAGCCCUACGUGAGCUGCGACCUGGACAAGUGCUACCAGUUC | |
| | GCCCUGGGACAGGGCACCACCCUGAACAACGUGCACAGCAACAA | |
| | CACUGUGCGGGACCGGACCCCAUACCGGACCCUGCUGAUGAACG | |
| | AGCUGGGCGUGCCCUUCCACCUGGGCACCAAGCAGGUGUGCAUC | |
| | GCCUGGAGCAGCAGCAGCUGCCACGACGGCAAGGCCUGGCUGCA | |
| | CGUGUGCAUUACCGGCGACGACAAGAACGCCACCGCCAGCUUCA | |
| | UCUACAACGGCAGGCUGGUGGACAGCGUGGUGAGCUGGAGCAAC | |
| | GACAUCCUGCGGACCCAGGACAGCGAGUGCGUGUGCAUCAACGG | |
| | CACCUGCACCGUGGUGAUGACUGACGGCAACGCCACCGGCAAGG | |
| | CCGACACCAAGAUCCUGUUCAUCGAGGAGGGGAAGAUCGUGCAC | |
| | ACCAGCAAGCUGUCUGGCAGCGCCCAGCACGUGGAGGAGUGCAG | |
| | CUGCUACCCUCGGUACCCCGGCGUGAGGUGCGUGUGCCGGGACA | |
| | ACUGGAAGGGCAGCAACCGGCCCAUCAUCGACAUCAACAUCAAG | |
| | GACCACAGCAUAGUGAGCAGAUACGUGUGCAGCGGUCUGGUGGG | |
| | CGACACUCCCCGGAAGAGCGACAGCAGCUCCAGCAGCCACUGCC | |
| | UGAACCCCAACAACGAGAAGGGUGACCACGGCGUGAAGGGCUGG | |
| | GCCUUCGACGACGGCAACGACGUGUGGAUGGGCCGGACCAUCAA | |
| | CGAGACCAGCAGACUGGGCUACGAGACCUUCAAGGUGGUGGAGG | |
| | GCUGGAGCAAUCCCAAGAGCAAGCUGCAGAUCAACCGGCAGGUG | |
| | AUCGUCGAUCGGGGCGAUCGGAGCGGCUACAGCGGCAUCUUCAG | |
| | CGUGGAGGGCAAGAGCUGCAUCAACCGGUGCUUCUACGUGGAGC | |
| | UGAUCCGGGGCCGGAAGGAGGAGACCGAGGUGCUGUGGACCAGC | |
| | AACAGCAUCGUGGUGUUCUGCGGCACCAGCGGCACCUACGGCAC | |
| | CGGAUCCUGGCCAGACGGCGCCAACCUGAGCCUGAUGCACAUC | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSVSLTISTICFFMQIAILITTVTLHFKQYEFNSPPNNQVM | 28 |
| amino acid | LCEPTIIERNMTEIVYLTNTTIEKEICPKPAEYRNWSKPQCGITGFAPFS | |
| sequence | KDNSIRLSAGGDIWVTREPYVSCDLDKCYQFALGQGTTLNNVHSNNT | |
| | VRDRTPYRTLLMNELGVPFHLGTKQVCIAWSSSSCHDGKAWLHVCIT | |
| | GDDKNATASFIYNGRLVDSVVSWSNDILRTQDSECVCINGTCTVVMT | |
| | DGNATGKADTKILFIEEGKIVHTSKLSGSAQHVEECSCYPRYPGVRCV | |
| | CRDNWKGSNRPIIDINIKDHSIVSRYVCSGLVGDTPRKSDSSSSSHCLN | |
| | PNNEKGDHGVKGWAFDDGNDVWMGRTINETSRLGYETFKVVEGWS | |
| | NPKSKLQINRQVIVDRGDRSGYSGIFSVEGKSCINRCFYVELIRGRKEE | |
| | TEVLWTSNSIVVFCGTSGTYGTGSWPDGANLSLMHI | |
| PolyA Tail | 100 nt | |

N1 Wisconsin D151G
SEQ ID NO: 47 consists of from 5′ end to 3′ end: 5′UTR SEQ ID NO: 30, mRNA ORF SEQ ID
NO: 56 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCCAACCAGAAGAUCAUCACCAUCGGCAGCAUCUGCAU | 56 |
| mRNA | GACCAUCGGCACCGCCAACCUGAUCCUGCAAAUCGGCAACAUCA | |
| (excluding the | UCAGCAUCUGGGUGAGCCACAGCAUCCAGAUCGGCAACCAGAGC | |
| stop codon) | CAGAUCGAGACCUGCAACAAGAGCGUGAUCACCUACGAGAACAA | |
| | CACCUGGGUGAACCAGACCUUCGUGAACAUCAGCAACACCAACA | |
| | GCGCCGCUCGGCAGUCAGUGGCCAGCGUGAAGCUGGCCGGCAAC | |
| | AGCAGCCUGUGCCCCGUUAGUGGCUGGGCCAUCUACAGCAAGGA | |
| | CAACAGCGUGCGGAUCGGCAGCAAGGGCGACGUGUUCGUGAUCC | |
| | GGGAGCCCUUCAUCAGCUGCAGCCCGCUUGAGUGCCGCACCUUC | |
| | UUCCUGACCCAGGGCGCUCUGCUGAACGACAAGCACAGCAACGG | |
| | CACCAUCAAGGGCCGGAGCCCCUAUCGGACCCUGAUGAGCUGCC | |
| | CCAUUGGCGAGGUGCCCAGCCCCUACAACAGCCGGUUCGAGUCU | |
| | GUGGCCUGGAGCGCCUCUGCCUGCCACGACGGCACCAACUGGCU | |
| | GACCAUCGGGAUCAGCGGACCCGAUAGCGGAGCAGUGGCCGUGC | |
| | UGAAGUACAACGGCAUCAUCACCGACACCAUCAAGAGCUGGCGG | |
| | AACAAGAUCCUGCGGACCCAGGAGAGCGAGUGCGCCUGCGUGAA | |
| | CGGCAGCUGCUUCACCAUCAUGACCGACGGCCCUAGCGACGGAC | |
| | AGGCCAGCUACAAGAUCUUCCGGAUCGAGAAGGGCAAGAUCAUC | |
| | AAGAGCGUGGAGAUGAAGGCACCCAACUACCACUACGAGGAGUG | |
| | CAGCUGCUACCCCGACAGCAGCGAGAUCACCUGCGUGUGCCGGG | |
| | ACAACUGGCACGGGAGCAACAGGCCCUGGGUGAGCUUCAACCAG | |
| | AACCUGGAGUACCAGAUGGGCUACAUCUGCAGCGGCGUGUUCGG | |
| | CGACAACCCACGGCCCAACGACAAGACUGGCAGCUGCGGUCCGG | |
| | UGAGCAGCAACGGCGCCAACGGCGUGAAGGGCUUCAGCUUCAAG | |
| | UACGGCAACGGCGUGUGGAUCGGCCGGACCAAGAGCAUCAGCAG | |
| | CCGGAAGGGCUUCGAGAUGAUCUGGGACCCCAACGGCUGGACCG | |
| | GCACCGACAACAAGUUCAGCAAGAAGCAGGACAUCGUGGGCAUC | |
| | AACGAGUGGAGCGGCUACAGCGGCAGCUUCGUGCAGCACCCCGA | |
| | GCUGACUGGCCUGAACUGCAUCCGGCCCUGCUUCUGGGUGGAAC | |
| | UGAUACGGGGACGGCCCGAGGAGAACACCAUCUGGACCAGCGGC | |
| | AGCAGCAUCAGCUUCUGCGGCGUGGACAGCGAUAUCGUGGGCUG | |
| | GAGCUGGCCAGACGGAGCCGAGCUGCCCUUCACCAUCGACAAG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSICMTIGTANLILQIGNIISIWVSHSIQIGNQSQIETCNKS | 62 |
| amino acid | VITYENNTWVNQTFVNISNTNSAARQSVASVKLAGNSSLCPVSGWAI | |
| sequence | YSKDNSVRIGSKGDVFVIREPFISCSPLECRTFFLTQGALLNDKHSNGTI | |
| | KGRSPYRTLMSCPIGEVPSPYNSRFESVAWSASACHDGTNWLTIGISG | |
| | PDSGAVAVLKYNGIITDTIKSWRNKILRTQESECACVNGSCFTIMTDG | |
| | PSDGQASYKIFRIEKGKIIKSVEMKAPNYHYEECSCYPDSSEITCVCRD | |
| | NWHGSNRPWVSFNQNLEYQMGYICSGVFGDNPRPNDKTGSCGPVSS | |
| | NGANGVKGFSFKYGNGVWIGRTKSISSRKGFEMIWDPNGWTGTDNK | |
| | FSKKQDIVGINEWSGYSGSFVQHPELTGLNCIRPCFWVELIRGRPEENT | |
| | IWTSGSSISFCGVDSDIVGWSWPDGAELPFTIDK | |
| PolyA Tail | 100 nt | |

N1_Wisconsin_E227D
SEQ ID NO: 51 consists of from 5′ end to 3′ end: 5′UTR SEQ ID NO: 30, mRNA ORF SEQ ID
NO: 57 and 3′UTR SEQ ID NO: 32.

| | | |
|---|---|---|
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGAACCCCAACCAGAAGAUCAUCACCAUCGGCAGCAUCUGCAU | 57 |
| mRNA | GACCAUCGGCACCGCCAACCUGAUCCUGCAAAUCGGCAACAUCA | |
| (excluding the | UCAGCAUCUGGGUGAGCCACAGCAUCCAGAUCGGCAACCAGAGC | |
| stop codon) | CAGAUCGAGACCUGCAACAAGAGCGUGAUCACCUACGAGAACAA | |
| | CACCUGGGUGAACCAGACCUUCGUGAACAUCAGCAACACCAACA | |
| | GCGCCGCUCGGCAGUCAGUGGCCAGCGUGAAGCUGGCCGGCAAC | |
| | AGCAGCCUGUGCCCCGUUAGUGGCUGGGCCAUCUACAGCAAGGA | |
| | CAACAGCGUGCGGAUCGGCAGCAAGGGCGACGUGUUCGUGAUCC | |
| | GGGAGCCCUUCAUCAGCUGCAGCCCGCUUGAGUGCCGCACCUUC | |
| | UUCCUGACCCAGGGCGCUCUGCUGAACGACAAGCACAGCAACGG | |
| | CACCAUCAAGGACCGGAGCCCCUAUCGGACCCUGAUGAGCUGCC | |
| | CCAUUGGCGAGGUGCCCAGCCCCUACAACAGCCGGUUCGAGUCU | |
| | GUGGCCUGGAGCGCCUCUGCCUGCCACGACGGCACCAACUGGCU | |
| | GACCAUCGGGAUCAGCGGACCCGAUAGCGGAGCAGUGGCCGUGC | |
| | UGAAGUACAACGGCAUCAUCACCGACACCAUCAAGAGCUGGCGG | |
| | AACAAGAUCCUGCGGACCCAGGACAGCGAGUGCGCCUGCGUGAA | |
| | CGGCAGCUGCUUCACCAUCAUGACCGACGGCCCUAGCGACGGAC | |
| | AGGCCAGCUACAAGAUCUUCCGGAUCGAGAAGGGCAAGAUCAUC | |
| | AAGAGCGUGGAGAUGAAGGCACCCAACUACCACUACGAGGAGUG | |
| | CAGCUGCUACCCCGACAGCAGCGAGAUCACCUGCGUGUGCCGGG | |
| | ACAACUGGCACGGGAGCAACAGGCCCUGGGUGAGCUUCAACCAG | |
| | AACCUGGAGUACCAGAUGGGCUACAUCUGCAGCGGCGUGUUCGG | |
| | CGACAACCCACGGCCCAACGACAAGACUGGCAGCUGCGGUCCGG | |
| | UGAGCAGCAACGGCGCCAACGGCGUGAAGGGCUUCAGCUUCAAG | |
| | UACGGCAACGGCGUGUGGAUCGGCCGGACCAAGAGCAUCAGCAG | |
| | CCGGAAGGGCUUCGAGAUGAUCUGGGACCCCAACGGCUGGACCG | |
| | GCACCGACAACAAGUUCAGCAAGAAGCAGGACAUCGUGGGCAUC | |
| | AACGAGUGGAGCGGCUACAGCGGCAGCUUCGUGCAGCACCCCGA | |
| | GCUGACUGGCCUGAACUGCAUCCGGCCCUGCUUCUGGGUGGAAC | |
| | UGAUACGGGGACGGCCCGAGGAGAACACCAUCUGGACCAGCGGC | |
| | AGCAGCAUCAGCUUCUGCGGCGUGGACAGCGAUAUCGUGGGCUG | |
| | GAGCUGGCCAGACGGAGCCGAGCUGCCCUUCACCAUCGACAAG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MNPNQKIITIGSICMTIGTANLILQIGNIISIWVSHSIQIGNQSQIETCNKS | 63 |
| amino acid | VITYENNTWVNQTFVNISNTNSAARQSVASVKLAGNSSLCPVSGWAI | |
| sequence | YSKDNSVRIGSKGDVFVIREPFISCSPLECRTFFLTQGALLNDKHSNGTI | |
| | KDRSPYRTLMSCPIGEVPSPYNSRFESVAWSASACHDGTNWLTIGISG | |
| | PDSGAVAVLKYNGIITDTIKSWRNKILRTQDSECACVNGSCFTIMTDG | |
| | PSDGQASYKIFRIEKGKIIKSVEMKAPNYHYEECSCYPDSSEITCVCRD | |
| | NWHGSNRPWVSFNQNLEYQMGYICSGVFGDNPRPNDKTGSCGPVSS | |
| | NGANGVKGFSFKYGNGVWIGRTKSISSRKGFEMIWDPNGWTGTDNK | |
| | FSKKQDIVGINEWSGYSGSFVQHPELTGLNCIRPCFWVELIRGRPEENT | |
| | IWTSGSSISFCGVDSDIVGWSWPDGAELPFTIDK | |
| PolyA Tail | 100 nt | |

B Phuket NA D151G
SEQ ID NO: 52 consists of from 5′ end to 3′ end: 5′UTR SEQ ID NO: 30, mRNA ORF SEQ ID
NO: 58 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUCCUGACCAG | 58 |
| mRNA | CGGAGGCGUGCUGCUGAGCCUGUACGUCAGCGCCAGCCUGAGCU | |
| (excluding the | ACCUGCUGUACAGCGACAUCCUGCUGAAGUUCAGCCGGACCGAG | |
| stop codon) | GUGACCGCUCCCAUCAUGCCCCUGGACUGCGCCAACGCCAGCAA | |
| | CGUGCAGGCCGUGAAUCGGAGCGCCACCAAGGGCGUGACUCCCC | |
| | UGCUGCCCGAGCCUGAGUGGACUUAUCCUCGGCUGAGCUGCCCA | |
| | GGCAGCACCUUCCAGAAGGCCCUGCUGAUCAGCCCACACCGGUU | |
| | CGGCGAGACCAAGGGCAACAGCGCUCCCCUGAUCAUCCGGGAGC | |
| | CCUUCAUCGCCUGCGGCCCCAAGGAGUGCAAGCACUUCGCCCUG | |
| | ACCCACUACGCUGCCCAACCCGGAGGCUACUACAACGGCACCAG | |
| | AGAGGGCCGGAACAAGCUGCGGCACCUGAUCAGCGUGAAGCUGG | |
| | GCAAGAUCCCCACCGUGGAGAACAGCAUCUUCCACAUGGCUGCU | |
| | UGGUCUGGAAGUGCUUGUCACGACGGCCGGGAGUGGACCUACAU | |
| | CGGCGUGGACGGCCCAGACAGCAACGCCCUGCUGAAGAUCAAGU | |
| | ACGGCGAGGCCUACACCGACACCUACCACAGCUACGCCAAGAAC | |
| | AUCCUGCGGACCCAGGAGAGCGCCUGCAACUGCAUCGGCGGCGA | |
| | CUGCUACCUGAUGAUCACCGACGGCCCAGCAUCUGGCAUCAGCG | |
| | AGUGCCGGUUCCUGAAGAUCCGGGAGGGCCGGAUCAUCAAGGAG | |
| | AUCUUCCCCACCGGGAGAGUGAAGCACACCGAGGAGUGCACCUG | |
| | CGGCUUCGCCAGCAACAAGACCAUCGAGUGCGCCUGCCGGGACA | |
| | ACAGCUACACCGCCAAGCGGCCCUUCGUGAAGCUGAACGUGGAG | |
| | ACCGACACCGCCGAGAUCCGGCUGAUGUGCACCAAGACCUACCU | |
| | GGACACCCCUCGGCCCAACGACGGAAGCAUCACCGGACCCUGCG | |
| | AGAGCGACGGGGACGAAGGAAGCGGCGGAAUCAAGGGCGGCUUC | |
| | GUGCACCAGCGGAUGGCCAGCAAGAUCGGCCGGUGGUACAGCCG | |
| | GACCAUGAGCAAGACCAAGCGGAUGGGCAUGGGCCUGUACGUGA | |
| | AGUACGACGGCGACCCCUGGACAGACAGCGAAGCCCUGGCCCUG | |
| | UCUGGCGUGAUGGUGAGCAUGGAGGAGCCCGGCUGGUACAGCUU | |
| | CGGCUUCGAGAUCAAGGACAAGAAGUGCGACGUGCCCUGCAUCG | |
| | GCAUCGAGAUGGUGCACGACGGCGGCAAGACCACCUGGCAUAGC | |
| | GCCGCAACCGCGAUCUACUGCCUGAUGGGCAGCGGCCAGCUGCU | |
| | GUGGGACACCGUGACCGGCGUGAACAUGACCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSRTEVTAPIM | 64 |
| amino acid | PLDCANASNVQAVNRSATKGVTPLLPEPEWTYPRLSCPGSTFQKALLI | |
| sequence | SPHRFGETKGNSAPLIIREPFIACGPKECKHEALTHYAAQPGGYYNGT | |
| | REGRNKLRHLISVKLGKIPTVENSIFHMAAWSGSACHDGREWTYIGV | |
| | DGPDSNALLKIKYGEAYTDTYHSYAKNILRTQESACNCIGGDCYLMIT | |
| | DGPASGISECRFLKIREGRIIKEIFPTGRVKHTEECTCGFASNKTIECAC | |
| | RDNSYTAKRPFVKLNVETDTAEIRLMCTKTYLDTPRPNDGSITGPCES | |
| | DGDEGSGGIKGGFVHQRMASKIGRWYSRTMSKTKRMGMGLYVKYD | |
| | GDPWTDSEALALSGVMVSMEEPGWYSFGFEIKDKKCDVPCIGIEMVH | |
| | DGGKTTWHSAATAIYCLMGSGQLLWDTVTGVNMTL | |
| PolyA Tail | 100 nt | |

B_NA_Phuket_2013_E227D
SEQ ID NO: 53 consists of from 5′ end to 3′ end: 5′UTR SEQ ID NO: 30, mRNA ORF SEQ ID
NO: 59 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUCCUGACCAG | 59 |
| mRNA | CGGAGGCGUGCUGCUGAGCCUGUACGUCAGCGCCAGCCUGAGCU | |
| (excluding the | ACCUGCUGUACAGCGACAUCCUGCUGAAGUUCAGCCGGACCGAG | |
| stop codon) | GUGACCGCUCCCAUCAUGCCCCUGGACUGCGCCAACGCCAGCAA | |
| | CGUGCAGGCCGUGAAUCGGAGCGCCACCAAGGGCGUGACUCCCC | |
| | UGCUGCCCGAGCCUGAGUGGACUUAUCCUCGGCUGAGCUGCCCA | |
| | GGCAGCACCUUCCAGAAGGCCCUGCUGAUCAGCCCACACCGGUU | |
| | CGGCGAGACCAAGGGCAACAGCGCUCCCCUGAUCAUCCGGGAGC | |
| | CCUUCAUCGCCUGCGGCCCCAAGGAGUGCAAGCACUUCGCCCUG | |
| | ACCCACUACGCUGCCCAACCCGGAGGCUACUACAACGGCACCAG | |
| | AGAGGACCGGAACAAGCUGCGGCACCUGAUCAGCGUGAAGCUGG | |
| | GCAAGAUCCCCACCGUGGAGAACAGCAUCUUCCACAUGGCUGCU | |
| | UGGUCUGGAAGUGCUUGUCACGACGGCCGGGAGUGGACCUACAU | |
| | CGGCGUGGACGGCCCAGACAGCAACGCCCUGCUGAAGAUCAAGU | |
| | ACGGCGAGGCCUACACCGACACCUACCACAGCUACGCCAAGAAC | |
| | AUCCUGCGGACCCAGGACAGCGCCUGCAACUGCAUCGGCGGCGA | |
| | CUGCUACCUGAUGAUCACCGACGGCCCAGCAUCUGGCAUCAGCG | |
| | AGUGCCGGUUCCUGAAGAUCCGGGAGGGCCGGAUCAUCAAGGAG | |
| | AUCUUCCCCACCGGGAGAGUGAAGCACACCGAGGAGUGCACCUG | |
| | CGGCUUCGCCAGCAACAAGACCAUCGAGUGCGCCUGCCGGGACA | |
| | ACAGCUACACCGCCAAGCGGCCCUUCGUGAAGCUGAACGUGGAG | |
| | ACCGACACCGCCGAGAUCCGGCUGAUGUGCACCAAGACCUACCU | |
| | GGACACCCCUCGGCCCAACGACGGAAGCAUCACCGGACCCUGCG | |
| | AGAGCGACGGGGACGAAGGAAGCGGCGGAAUCAAGGGCGGCUUC | |
| | GUGCACCAGCGGAUGGCCAGCAAGAUCGGCCGGUGGUACAGCCG | |
| | GACCAUGAGCAAGACCAAGCGGAUGGGCAUGGGCCUGUACGUGA | |
| | AGUACGACGGCGACCCCUGGACAGACAGCGAAGCCCUGGCCCUG | |
| | UCUGGCGUGAUGGUGAGCAUGGAGGAGCCCGGCUGGUACAGCUU | |
| | CGGCUUCGAGAUCAAGGACAAGAAGUGCGACGUGCCCUGCAUCG | |
| | GCAUCGAGAUGGUGCACGACGGCGGCAAGACCACCUGGCAUAGC | |
| | GCCGCAACCGCGAUCUACUGCCUGAUGGGCAGCGGCCAGCUGCU | |
| | GUGGGACACCGUGACCGGCGUGAACAUGACCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSRTEVTAPIM | 65 |
| amino acid | PLDCANASNVQAVNRSATKGVTPLLPEPEWTYPRLSCPGSTFQKALLI | |
| sequence | SPHRFGETKGNSAPLIIREPFIACGPKECKHEALTHYAAQPGGYYNGT | |
| | REDRNKLRHLISVKLGKIPTVENSIFHMAAWSGSACHDGREWTYIGV | |
| | DGPDSNALLKIKYGEAYTDTYHSYAKNILRTQDSACNCIGGDCYLMI | |
| | TDGPASGISECRFLKIREGRIIKEIFPTGRVKHTEECTCGFASNKTIECA | |
| | CRDNSYTAKRPFVKLNVETDTAEIRLMCTKTYLDTPRPNDGSITGPCE | |
| | SDGDEGSGGIKGGFVHQRMASKIGRWYSRTMSKTKRMGMGLYVKY | |
| | DGDPWTDSEALALSGVMVSMEEPGWYSFGFEIKDKKCDVPCIGIEMV | |
| | HDGGKTTWHSAATAIYCLMGSGQLLWDTVTGVNMTL | |
| PolyA Tail | 100 nt | |

B Washington NA D151G
SEQ ID NO: 54 consists of from 5′ end to 3′ end: 5′UTR SEQ ID NO: 30, mRNA ORF SEQ ID
NO: 60 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUUCUGACCAG | 60 |
| mRNA | CGGAGGCGUGCUGCUGAGCCUGUACGUGAGCGCCAGCCUGAGCU | |
| (excluding the | ACCUGCUGUACAGCGACAUCCUGCUGAAGUUCAGCCCCACCGAG | |
| stop codon) | AUCACCGCACCCACCAUGCCCCUGGACUGCGCCAACGCCAGCAAC | |
| | GUGCAGGCCGUGAACCGGAGCGCCACAAAGGGCGUGACCCUGCU | |
| | GCUGCCCGAGCCAGAGUGGACAUAUCCUCGGCUGAGCUGCCCUG | |
| | GCAGCACCUUCCAGAAGGCCCUGCUGAUCAGCCCACACCGGUUC | |
| | GGCGAGACCAAGGGCAACAGCGCACCCCUGAUCAUCCGGGAGCC | |
| | CUUCGUGGCCUGUGGCCCCAACGAGUGCAAGCACUUCGCCCUGA | |
| | CACACUACGCUGCUCAGCCCGGUGGCUACUACAACGGCACCCGG | |
| | GGUGGCCGGAACAAGCUGCGGCACCUGAUCAGCGUGAAGCUGGG | |
| | CAAGAUCCCCACCGUGGAGAACAGCAUCUUCCACAUGGCCGCCU | |
| | GGUCAGGAAGCGCCUGCCACGACGGCAAGGAGUGGACCUACAUC | |
| | GGCGUGGACGGCCCUGACAACAACGCCCUGCUGAAGGUGAAGUA | |
| | CGGCGAGGCCUACACCGACACCUACCACAGCUACGCCAACAACA | |
| | UCCUGCGGACCCAGGAGAGCGCCUGCAACUGCAUCGGCGGCAAC | |
| | UGCUACCUGAUGAUCACCGACGGCAGCGCUUCUGGCGUGAGCGA | |
| | GUGCCGGUUCCUGAAGAUCCGGGAGGGCCGGAUCAUCAAGGAGA | |
| | UCUUUCCCACCGGCCGGGUGAAGCACACCGAGGAGUGCACCUGC | |
| | GGCUUCGCCAGCAACAAGACCAUCGAGUGCGCCUGCCGGGACAA | |
| | UCGGUACACCGCCAAGCGGCCCUUCGUGAAGCUGAACGUGGAGA | |
| | CCGACACCGCCGAGAUCCGGCUGAUGUGCACCGACACUUAUCUG | |
| | GACACCCCUCGGCCUAACGACGGCAGCAUCACCGGCCCUUGCGA | |
| | GAGCGACGGCGACAAGGGAAGCGGCGGCAUCAAGGGCGGUUUCG | |
| | UGCACCAGCGGAUGAAGAGCAAGAUCGGCCGGUGGUACAGCCGG | |
| | ACCAUGAGCAAGACCGAGCGGAUGGGCAUGGGCCUGUACGUAAA | |
| | GUACGGAGGGGAUCCCUGGGCUGACAGCGACGCCCUGACCUUCA | |
| | GCGGCGUGAUGGUGAGCAUGAAGGAGCCCGGCUGGUACAGCUUC | |
| | GGCUUCGAGAUCAAGGACAAGAAGUGCGACGUGCCCUGCAUCGG | |
| | CAUCGAGAUGGUGCACGACGGCGGCAAGGAGACCUGGCACUCUG | |
| | CCGCCACUGCCAUCUACUGCCUGAUGGGCAGCGGCCAGCUGCUG | |
| | UGGGACACCGUGACCGGCGUGGACAUGGCCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSPTEITAPTM | 66 |
| amino acid | PLDCANASNVQAVNRSATKGVTLLLPEPEWTYPRLSCPGSTFQKALLI | |
| sequence | SPHRFGETKGNSAPLIIREPFVACGPNECKHEALTHYAAQPGGYYNGT | |
| | RGGRNKLRHLISVKLGKIPTVENSIFHMAAWSGSACHDGKEWTYIGV | |
| | DGPDNNALLKVKYGEAYTDTYHSYANNILRTQESACNCIGGNCYLMI | |
| | TDGSASGVSECRFLKIREGRIIKEIFPTGRVKHTEECTCGFASNKTIECA | |
| | CRDNRYTAKRPFVKLNVETDTAEIRLMCTDTYLDTPRPNDGSITGPCE | |
| | SDGDKGSGGIKGGFVHQRMKSKIGRWYSRTMSKTERMGMGLYVKY | |
| | GGDPWADSDALTFSGVMVSMKEPGWYSFGFEIKDKKCDVPCIGIEM | |
| | VHDGGKETWHSAATAIYCLMGSGQLLWDTVTGVDMAL | |
| PolyA Tail | 100 nt | |

B_NA_Washington_2019_E227D
SEQ ID NO: 55 consists of from 5′ end to 3′ end: 5′UTR SEQ ID NO: 30, mRNA ORF SEQ ID
NO: 61 and 3′UTR SEQ ID NO: 32.

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACC | 30 |
| | CCGGCGCCGCCACC | |
| ORF of | AUGCUGCCCAGCACCAUCCAGACCCUGACCCUGUUUCUGACCAG | 61 |
| mRNA | CGGAGGCGUGCUGCUGAGCCUGUACGUGAGCGCCAGCCUGAGCU | |
| (excluding the | ACCUGCUGUACAGCGACAUCCUGCUGAAGUUCAGCCCCACCGAG | |
| stop codon) | AUCACCGCACCCACCAUGCCCCUGGACUGCGCCAACGCCAGCAAC | |
| | GUGCAGGCCGUGAACCGGAGCGCCACAAAGGGCGUGACCCUGCU | |
| | GCUGCCCGAGCCAGAGUGGACAUAUCCUCGGCUGAGCUGCCCUG | |
| | GCAGCACCUUCCAGAAGGCCCUGCUGAUCAGCCCACACCGGUUC | |
| | GGCGAGACCAAGGGCAACAGCGCACCCCUGAUCAUCCGGGAGCC | |
| | CUUCGUGGCCUGUGGCCCCAACGAGUGCAAGCACUUCGCCCUGA | |
| | CACACUACGCUGCUCAGCCCGGUGGCUACUACAACGGCACCCGG | |
| | GGUGAUCGGAACAAGCUGCGGCACCUGAUCAGCGUGAAGCUGGG | |
| | CAAGAUCCCCACCGUGGAGAACAGCAUCUUCCACAUGGCCGCCU | |
| | GGUCAGGAAGCGCCUGCCACGACGGCAAGGAGUGGACCUACAUC | |
| | GGCGUGGACGGCCCUGACAACAACGCCCUGCUGAAGGUGAAGUA | |
| | CGGCGAGGCCUACACCGACACCUACCACAGCUACGCCAACAACA | |
| | UCCUGCGGACCCAGGACAGCGCCUGCAACUGCAUCGGCGGCAAC | |
| | UGCUACCUGAUGAUCACCGACGGCAGCGCUUCUGGCGUGAGCGA | |
| | GUGCCGGUUCCUGAAGAUCCGGGAGGGCCGGAUCAUCAAGGAGA | |
| | UCUUUCCCACCGGCCGGGUGAAGCACACCGAGGAGUGCACCUGC | |
| | GGCUUCGCCAGCAACAAGACCAUCGAGUGCGCCUGCCGGGACAA | |
| | UCGGUACACCGCCAAGCGGCCCUUCGUGAAGCUGAACGUGGAGA | |
| | CCGACACCGCCGAGAUCCGGCUGAUGUGCACCGACACUUAUCUG | |
| | GACACCCCUCGGCCUAACGACGGCAGCAUCACCGGCCCUUGCGA | |
| | GAGCGACGGCGACAAGGGAAGCGGCGGCAUCAAGGGCGGUUUCG | |
| | UGCACCAGCGGAUGAAGAGCAAGAUCGGCCGGUGGUACAGCCGG | |
| | ACCAUGAGCAAGACCGAGCGGAUGGGCAUGGGCCUGUACGUAAA | |
| | GUACGGAGGGGAUCCCUGGGCUGACAGCGACGCCCUGACCUUCA | |
| | GCGGCGUGAUGGUGAGCAUGAAGGAGCCCGGCUGGUACAGCUUC | |
| | GGCUUCGAGAUCAAGGACAAGAAGUGCGACGUGCCCUGCAUCGG | |
| | CAUCGAGAUGGUGCACGACGGCGGCAAGGAGACCUGGCACUCUG | |
| | CCGCCACUGCCAUCUACUGCCUGAUGGGCAGCGGCCAGCUGCUG | |
| | UGGGACACCGUGACCGGCGUGGACAUGGCCCUG | |
| 3′ UTR | UGAUAAUAGGCUGGAGCCUCGGUGGCCUAGCUUCUUGCCCCUUG | 32 |
| | GGCCUCCCCCCAGCCCCUCCUCCCCUUCCUGCACCCGUACCCCCG | |
| | UGGUCUUUGAAUAAAGUCUGAGUGGGCGGC | |
| Corresponding | MLPSTIQTLTLFLTSGGVLLSLYVSASLSYLLYSDILLKFSPTEITAPTM | 67 |
| amino acid | PLDCANASNVQAVNRSATKGVTLLLPEPEWTYPRLSCPGSTFQKALLI | |
| sequence | SPHRFGETKGNSAPLIIREPFVACGPNECKHEALTHYAAQPGGYYNGT | |
| | RGDRNKLRHLISVKLGKIPTVENSIFHMAAWSGSACHDGKEWTYIGV | |
| | DGPDNNALLKVKYGEAYTDTYHSYANNILRTQDSACNCIGGNCYLM | |
| | ITDGSASGVSECRFLKIREGRIIKEIFPTGRVKHTEECTCGFASNKTIEC | |
| | ACRDNRYTAKRPFVKLNVETDTAEIRLMCTDTYLDTPRPNDGSITGPC | |
| | ESDGDKGSGGIKGGFVHQRMKSKIGRWYSRTMSKTERMGMGLYVK | |
| | YGGDPWADSDALTFSGVMVSMKEPGWYSFGFEIKDKKCDVPCIGIE | |
| | MVHDGGKETWHSAATAIYCLMGSGQLLWDTVTGVDMAL | |
| PolyA Tail | 100 nt | |

Any of the mRNA sequences described herein may include a 5′ UTR and/or a 3′ UTR. The UTR sequences may be selected from the following sequences, or other known UTR sequences may be used. It should also be understood that any of the mRNAs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 29)
GGGAAAUAAGAGAGAAAAGAAGAGUAAGAAGAAAUAUAAGAGCCACC
5′ UTR:
(SEQ ID NO: 30)
GGGAAAUAAGAGAGAAAAGAAGAGUAAGAAGAAAUAUAAGACCCCGGCG
CCGCCACC
3′ UTR:
(SEQ ID NO: 31)
UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC
3′ UTR:
(SEQ ID NO: 32)
UGAUAAUAGGCUGGAGCCUCGGUGGCCUAGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC

### Embodiments

1. A combination vaccine, comprising a first messenger ribonucleic acid (mRNA) polynucleotide having an open reading frame (ORF) encoding a first respiratory virus antigenic polypeptide from an influenza virus; and a second mRNA polynucleotide having an ORF encoding a second respiratory virus antigenic polypeptide from a coronavirus.
2. The vaccine of embodiment 1, wherein the vaccine comprises at least 2 mRNA polynucleotides having an ORF encoding a first respiratory virus antigenic polypeptide an influenza virus.
3. The vaccine of embodiment 1, wherein the vaccine comprises at least 2 mRNA polynucleotides having an ORF encoding a second respiratory virus antigenic polypeptide from a coronavirus.
4. The vaccine of any one of embodiments 1-8, wherein the vaccine comprises less than 15 mRNA polynucleotides.
5. The vaccine of embodiment 4, wherein the vaccine comprises 3-10 mRNA polynucleotides.
6. The vaccine of embodiment 4, wherein the vaccine comprises 4-10 mRNA polynucleotides.
7. The vaccine of embodiment 4, wherein the vaccine comprises 5-10 mRNA polynucleotides.
8. The vaccine of embodiment 4, wherein the vaccine comprises 8-9 mRNA polynucleotides.
9. The vaccine of any one of embodiments 1-8, wherein the first and second mRNA polynucleotides are present in the combination vaccine in a ratio of 1:1.
10. The vaccine of any one of embodiments 1-9, wherein the vaccine comprises at least two mRNA polynucleotides encoding influenza virus antigenic polypeptides.
11. The vaccine of any one of embodiments 1-10, wherein the vaccine comprises at least three mRNA polynucleotides encoding influenza virus antigenic polypeptides.
12. The vaccine of any one of embodiments 1-11, wherein the vaccine comprises at least four mRNA polynucleotides encoding influenza virus antigenic polypeptides.
13. The vaccine of any one of embodiments 1-12, wherein the vaccine comprises at least two mRNA polynucleotides encoding coronavirus antigenic polypeptides.
14. The vaccine of any one of embodiments 1-13, wherein the combination vaccine comprises a ratio of mRNA polynucleotides encoding respiratory virus antigenic polypeptides of 4:1 from the first viral family to the second viral family.
15. The vaccine of any one of embodiments 1-13, wherein the combination vaccine comprises a ratio of mRNA polynucleotides encoding respiratory virus antigenic polypeptides of 3:1 from the first viral family to the second viral family.
16. The vaccine of any one of embodiments 1-13, wherein the combination vaccine comprises a ratio of mRNA polynucleotides encoding respiratory virus antigenic polypeptides of 5:1 from the first viral family to the second viral family.
17. The vaccine of any one of embodiments 1-13, wherein the combination vaccine comprises a ratio of mRNA polynucleotides encoding respiratory virus antigenic polypeptides of 2:1 from the first viral family to the second viral family.
18. The vaccine of any one of embodiments 1-13, wherein the combination vaccine comprises a ratio of mRNA polynucleotides encoding respiratory virus antigenic polypeptides of 4:2 from the first viral family to the second viral family.
19. The vaccine of any one of embodiments 1-13, wherein the combination vaccine comprises a ratio of mRNA polynucleotides encoding respiratory virus antigenic polypeptides of 1:2 from the first viral family to the second viral family to the third viral family.
20. The vaccine of any one of embodiments 1-19, wherein the combination vaccine is a multivalent RNA composition produced by the method comprising:
   (a) combining a linearized first DNA molecule encoding the first mRNA polynucleotide and a linearized second DNA molecule encoding the second mRNA polynucleotide into a single reaction vessel, wherein the first DNA molecule and the second DNA molecule are obtained from different sources; and
   (b) simultaneously in vitro transcribing the linearized first DNA molecule and the linearized second DNA molecule to obtain a multivalent RNA composition.
21. The vaccine of embodiment 20, wherein the different sources are a first and second bacterial cell culture and wherein the first and second bacterial cell culture are not co-cultured.
22. The vaccine of embodiment 20, wherein the amounts of the first and second DNA molecules present in the reaction mixture prior to the start of the IVT have been normalized.
23. The vaccine of any one of embodiments 1-22, wherein the combination vaccine is a multivalent RNA composition, wherein the multivalent RNA composition comprises greater than 40% polyA-tailed RNAs.
24. The vaccine of any one of embodiments 1-23, wherein the combination vaccine is a multivalent RNA composition wherein each of the first and second mRNA polynucleotides is different in length from one another by at least 100 nucleotides.
25. The vaccine of any one of embodiments 1-24, wherein the combination vaccine is a multivalent RNA composition wherein each of the first and second (and optionally, third, fourth, fifth, sixth, seventh, eighth, ninth, tenth, eleventh, twelfth, thirteenth, fourteen or fifteenth) mRNA polynucleotides comprises one or more non-coding sequence in an untranslated region (UTR), optionally a 5′ UTR or 3′ UTR.
26. The vaccine of embodiment 25, wherein the non-coding sequence is positioned in a 3′ UTR of an mRNA, upstream of the polyA tail of the mRNA.
27. The vaccine of embodiment 25, wherein the non-coding sequence is positioned in a 3′ UTR of an mRNA, downstream of the polyA tail of the mRNA.
28. The vaccine of embodiment 25, wherein the non-coding sequence is positioned in a 3′ UTR of an mRNA between the last codon of the ORF of the mRNA and the first “A” of the polyA tail of the mRNA.
29. The vaccine of embodiment 25, wherein the non-coding sequence comprises between 1 and 10 nucleotides).
30. The vaccine of any one of embodiments 25-29, wherein the non-coding sequence comprises one or more RNAse cleavage sites.
31. The vaccine of embodiment 30, wherein the RNAse cleavage site is an RNase H cleavage site.
32. The vaccine of any one of embodiments 1-31, wherein each of the mRNA polynucleotides in the combination vaccine is complementary with and does not interfere with each other mRNA polynucleotide in the combination vaccine.
33. The vaccine of any one of embodiments 1-32, wherein at least one of the respiratory virus antigenic polypeptides is derived from a naturally occurring antigen.
34. The vaccine of any one of embodiments 1-32, wherein at least one of the respiratory virus antigenic polypeptides is a stabilized version of a naturally occurring antigen.
35. The vaccine of any one of embodiments 1-32, wherein at least one of the respiratory virus antigenic polypeptides is a non-naturally occurring antigen.
36. The vaccine of any one of embodiments 1-33, wherein the vaccine further comprises an mRNA polynucleotide encoding a variant respiratory virus antigenic polypeptide, wherein the variant is a variant of any one of the first or second respiratory virus antigenic polypeptides.
37. The vaccine of any one of embodiments 1-36, wherein the second respiratory virus antigenic polypeptide is selected from the group consisting of MERS-CoV, SARS-CoV, SARS-CoV-2, HCoV-OC43, HCoV-229E, HCoV-NL63, HCoV-NL, HCoV-NH and HCoV-HKU1.
38. The vaccine of any one of embodiments 1-37, wherein the first respiratory virus antigenic polypeptide is influenza HA and/or influenza NA.
39. The vaccine of any one of embodiments 1-38, wherein the antigenic polypeptides include a Fusion (F) protein, a spike (S) protein, and a hemagglutinin antigen (HA).
40. The vaccine of any one of embodiments 1-39, further comprising at least one lipid nanoparticle (LNP).
41. The vaccine of embodiment 40, wherein the LNP comprises a molar ratio of 20-60% ionizable amino lipid, 5-25% non-cationic lipid, 25-55% sterol, and 0.5-15% PEG-modified lipid.
42. A method for vaccinating a subject, comprising:
   administering to the subject a combination vaccine, wherein the combination vaccine comprises a first messenger ribonucleic acid (mRNA) polynucleotide having an open reading frame (ORF) encoding a first respiratory virus antigenic polypeptide from an influenza virus; and a second mRNA polynucleotide having an ORF encoding a second respiratory virus antigenic polypeptide from a coronavirus.
43. The method of embodiment 42, wherein the subject is 65 years of age or older.
44. The method of embodiment 42, wherein the subject is under 18 years of age.
45. The method of embodiment 42, wherein the method prevents a respiratory infection in the subject.
46. The method of embodiment 42, wherein the method reduces the severity of a respiratory infection in the subject.
47. The method of embodiment 42, wherein the subject is seronegative for at least one of the antigenic polypeptides.
48. The method of embodiment 42, wherein the subject is seronegative for all of the antigenic polypeptides.
49. The method of embodiment 42, wherein the subject is seropositive for at least one of the antigenic polypeptides.
50. The method of embodiment 42, wherein the subject is seropositive for all of the antigenic polypeptides.
51. The method of any one of embodiments 42-50, further comprising administering a booster vaccine.
52. The method of embodiment 51, wherein the booster vaccine is administered between 3 weeks and 1 year after the combination vaccine.
53. The method of embodiment 51 or 52, wherein the booster vaccine comprises at least one mRNA polynucleotide having an ORF encoding the first or second respiratory virus antigenic polypeptides.
54. The method of embodiment 51 or 52, wherein the booster vaccine comprises at least one mRNA polynucleotide having an ORF encoding each of the first and second respiratory virus antigenic polypeptides.
55. The method of embodiment 51 or 52, wherein the booster vaccine comprises at least one mRNA polynucleotide having an ORF encoding a variant of the first or second respiratory virus antigenic polypeptides.
56. The method of any one of embodiments 42-55, wherein the combination vaccine is a seasonal booster vaccine.
57. The method of any one of embodiments 42-56, wherein the combination vaccine is a vaccine of any one of embodiments 1-41.
58. A method of preventing or reducing the severity of a respiratory infection by administering the vaccine of any one of embodiments 1-41 to a subject in an effective amount to prevent infection or reduce the severity of a respiratory infection in the subject based on a single dose or single dose with a booster.
59. The method of any one of embodiments 42-58, wherein the combination vaccine is administered to the subject in a dose of 20 μg or 50 μg.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the embodiments and/or claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A combination vaccine, comprising
a first messenger ribonucleic acid (mRNA) polynucleotide comprising an open reading frame (ORF) encoding a first respiratory virus antigenic polypeptide, wherein the first respiratory virus antigenic polypeptide is an influenza virus antigen; and
a second mRNA polynucleotide comprising an ORF encoding a second respiratory virus antigenic polypeptide from a coronavirus;
and a lipid nanoparticle.

2. A combination vaccine, comprising a first messenger ribonucleic acid (mRNA) polynucleotide comprising an open reading frame (ORF) encoding a first respiratory virus antigenic polypeptide, wherein the first respiratory virus antigenic polypeptide is an influenza virus antigen;
a second mRNA polynucleotide comprising an ORF encoding a second respiratory virus antigenic polypeptide from a second influenza virus;
a third mRNA polynucleotide comprising an ORF encoding a third respiratory virus antigenic polypeptide from a third influenza virus;
a fourth mRNA polynucleotide comprising an ORF encoding a fourth respiratory virus antigenic polypeptide from a fourth influenza virus;
a fifth mRNA polynucleotide comprising an ORF encoding a fifth respiratory virus antigenic polypeptide from a first coronavirus;
a sixth mRNA polynucleotide comprising an ORF encoding a sixth respiratory virus antigenic polypeptide from a second coronavirus;
and a lipid nanoparticle.

3. The combination vaccine of claim 2, wherein the first, second, third and fourth viruses are selected from influenza A viruses and influenza B viruses.

4. The combination vaccine of claim 1 or 2, wherein the coronavirus, first coronavirus, and/or second coronavirus is a betacoronavirus.

5. The combination vaccine of claim 1 or 2, wherein the coronavirus, first coronavirus, and/or second coronavirus is selected from the group consisting of MERS-CoV, SARS-CoV, SARS-CoV-2, HCoV-OC43, HCoV-229E, HCoV-NL63, HCoV-NL, HCoV-NH and HCoV-HKU1.

6. The combination vaccine of claim 1 or 2, wherein the first respiratory virus antigenic polypeptide is from an influenza virus B.

7. The combination vaccine of claim 1 or 2, wherein the first respiratory virus antigenic polypeptide is from an influenza virus A.

8. The combination vaccine of claim 1 or 2, wherein the first respiratory virus antigenic polypeptide is hemagglutinin antigen (HA) or a neuraminidase antigen (NA).

9. The combination vaccine of claim 1 or 2, wherein the second respiratory virus antigenic polypeptide is from a SARS-CoV.

10. The combination vaccine of claim 1 or 2, wherein the second respiratory virus antigenic polypeptide is from SARS-CoV-2.

11. The combination vaccine of claim 1 or 2, wherein the second respiratory virus antigenic polypeptide is from a non-SARS human coronavirus (HCoV).

12. The combination vaccine of any one of claims 1-11, wherein the vaccine comprises at least 2 mRNA polynucleotides comprising an ORF encoding an influenza virus antigen.

13. The combination vaccine of any one of claims 1-12, wherein the vaccine comprises 2-4 mRNA polynucleotides comprising an ORF encoding an influenza virus antigen.

14. The combination vaccine of any one of claims 1-12, wherein the vaccine comprises at least 2 mRNA polynucleotides comprising an ORF encoding a respiratory virus antigenic polypeptide from a coronavirus.

15. The combination vaccine of any one of claims 1-14, wherein the vaccine comprises less than 15 mRNA polynucleotides.

16. The combination vaccine of claim 15, wherein the vaccine comprises 3-10 mRNA polynucleotides.

17. The combination vaccine of claim 15, wherein the vaccine comprises 4-10 mRNA polynucleotides.

18. The combination vaccine of claim 15, wherein the vaccine comprises 5-10 mRNA polynucleotides.

19. The combination vaccine of claim 15, wherein the vaccine comprises 8-9 mRNA polynucleotides.

20. The combination vaccine of any one of claims 1-19, wherein the vaccine comprises at least three mRNA polynucleotides encoding influenza virus antigenic polypeptides.

21. The combination vaccine of claim 20, wherein the vaccine comprises at least eight mRNA polynucleotides encoding influenza virus antigenic polypeptides.

22. The combination vaccine of claim 20, wherein the vaccine comprises at least two mRNA polynucleotides encoding coronavirus antigenic polypeptides.

23. The combination vaccine of any one of claims 1-22, wherein the first and second mRNA polynucleotides are present in the combination vaccine in a ratio of 1:1.

24. The combination vaccine of any one of claims 1-22, wherein the combination vaccine comprises a ratio of mRNA polynucleotides encoding respiratory virus antigenic polypeptides of 4:1 from the influenza virus to the coronavirus.

25. The combination vaccine of any one of claims 1-22, wherein the combination vaccine comprises a ratio of mRNA polynucleotides encoding respiratory virus antigenic polypeptides of 3:1 from the influenza virus to the coronavirus.

26. The combination vaccine of any one of claims 1-22, wherein the combination vaccine comprises a ratio of mRNA polynucleotides encoding respiratory virus antigenic polypeptides of 2:1 from the influenza virus to the coronavirus.

27. The combination vaccine of any one of claims 1-22, wherein the combination vaccine comprises a ratio of mRNA polynucleotides encoding respiratory virus antigenic polypeptides of 5:1 from the influenza virus to the coronavirus.

28. The combination vaccine of any one of claims 1-22, wherein the combination vaccine comprises a ratio of mRNA polynucleotides encoding respiratory virus antigenic polypeptides of 4:2 from the influenza virus to the coronavirus.

29. The combination vaccine of any one of claims 1-22, wherein the combination vaccine comprises a ratio of mRNA polynucleotides encoding respiratory virus antigenic polypeptides of 1:2 from the influenza virus to the coronavirus.

30. The combination vaccine of any one of claims 1-22, wherein the combination vaccine comprises a ratio of mRNA polynucleotides encoding respiratory virus antigenic polypeptides of 8:2 from the first virus to the second virus.

31. The combination vaccine of any one of claims 1-22, wherein the combination vaccine comprises a ratio of mRNA polynucleotides encoding respiratory virus antigenic polypeptides of 8:1 from the first virus to the second virus.

32. The combination vaccine claim 31, wherein the respiratory virus antigenic polypeptides of the first virus comprise HAs and NAs, in a ratio of 4:4.

33. The combination vaccine of any one of claims 1-32, wherein each of the mRNA polynucleotides in the combination vaccine is complementary with and does not interfere with each other mRNA polynucleotide in the combination vaccine.

34. The combination vaccine of any one of claims 1-32, wherein at least one of the respiratory virus antigenic polypeptides is derived from a naturally occurring antigen.

35. The combination vaccine of any one of claims 1-32, wherein at least one of the respiratory virus antigenic polypeptides is a stabilized version of a naturally occurring antigen.

36. The combination vaccine of any one of claims 1-32, wherein at least one of the respiratory virus antigenic polypeptides is a non-naturally occurring antigen.

37. The combination vaccine of any one of claims 1-36, wherein the vaccine further comprises an mRNA polynucleotide encoding a structurally altered variant respiratory virus antigenic polypeptide, wherein the structurally altered variant is a structurally altered variant of any one of the first or second respiratory virus antigenic polypeptides.

38. The combination vaccine of any one of claims 1-36, wherein at least one of the first and second mRNA polynucleotides is polycistronic.

39. The combination vaccine of any one of claims 1-36, wherein each of the first and second mRNA polynucleotides is polycistronic.

40. A multivalent RNA composition, comprising a first messenger ribonucleic acid (mRNA) polynucleotide comprising an open reading frame (ORF) encoding a first respiratory virus antigenic polypeptide, from a first virus; and
a second mRNA polynucleotide comprising an ORF encoding a second respiratory virus antigenic polypeptide from a coronavirus;
wherein the multivalent RNA composition comprises greater than 40% polyA-tailed RNAs and/or the first and/or second mRNA polynucleotides is different in length from one another by at least 100 nucleotides.

41. The multivalent RNA composition of claim 40, wherein the composition is produced by a method comprising:
(a) combining a linearized first DNA molecule encoding the first mRNA polynucleotide and a linearized second DNA molecule encoding the second mRNA polynucleotide into a single reaction vessel, wherein the first DNA molecule and the second DNA molecule are obtained from different sources; and
(b) simultaneously in vitro transcribing the linearized first DNA molecule and the linearized second DNA molecule to obtain a multivalent RNA composition.

42. The multivalent RNA composition of claim 41, wherein the different sources are a first and second bacterial cell culture and wherein the first and second bacterial cell culture are not co-cultured.

43. The multivalent RNA composition of claim 42, wherein the amounts of the first and second DNA molecules present in the reaction mixture prior to the start of the IVT have been normalized.

44. The multivalent RNA composition of claim 40, wherein the coronavirus is selected from the group consisting of MERS-CoV, SARS-CoV, SARS-CoV-2, HCoV-OC43, HCoV-229E, HCoV-NL63, HCoV-NL, HCoV-NH and HCoV-HKU1.

45. A multivalent RNA composition, comprising 2-15 mRNA polynucleotides, each comprising a distinct open reading frame (ORF) encoding a respiratory virus antigenic polypeptide, wherein at least one respiratory virus antigenic polypeptide is an influenza virus and at least one respiratory virus antigenic polypeptide is a coronavirus, and wherein each mRNA polynucleotide comprises one or more non-coding sequence in an untranslated region (UTR), optionally a 5′ UTR or 3′ UTR.

46. The multivalent RNA composition of claim 45, wherein the non-coding sequence is positioned in a 3′ UTR of an mRNA, upstream of the polyA tail of the mRNA.

47. The multivalent RNA composition of claim 45, wherein the non-coding sequence is positioned in a 3′ UTR of an mRNA, downstream of the polyA tail of the mRNA.

48. The multivalent RNA composition of claim 45, wherein the non-coding sequence is positioned in a 3′ UTR of an mRNA between the last codon of the ORF of the mRNA and the first “A” of the polyA tail of the mRNA.

49. The multivalent RNA composition of claim 45, wherein the non-coding sequence comprises between 1 and 10 nucleotides.

50. The multivalent RNA composition of any one of claims 45-49, wherein the non-coding sequence comprises one or more RNAse cleavage sites.

51. The multivalent RNA composition of claim 50, wherein the RNAse cleavage site is an RNase H cleavage site.

52. The multivalent RNA composition of claim 45, wherein the coronavirus antigen is selected from the group consisting of MERS-CoV, SARS-CoV, SARS-CoV-2, HCoV-OC43, HCoV-229E, HCoV-NL63, HCoV-NL, HCoV-NH and HCoV-HKU1.

53. A multivalent RNA composition, comprising a first messenger ribonucleic acid (mRNA) polynucleotide comprising an open reading frame (ORF) encoding a first respiratory virus antigenic polypeptide, from an influenza virus;
a second mRNA polynucleotide comprising an ORF encoding a second respiratory virus antigenic polypeptide from a coronavirus; and
wherein at least one of the respiratory virus antigenic polypeptides is derived from a naturally occurring antigen or a stabilized version of a naturally occurring antigen and further comprising an mRNA polynucleotide encoding a structurally altered variant respiratory virus antigenic polypeptide, wherein the structurally altered variant is a structurally altered variant of any one of the first or second respiratory virus antigenic polypeptides.

54. The multivalent RNA composition of claim 53, wherein the coronavirus is selected from the group consisting of MERS-CoV, SARS-CoV, SARS-CoV-2, HCoV-OC43, HCoV-229E, HCoV-NL63, HCoV-NL, HCoV-NH and HCoV-HKU1.

55. The multivalent RNA composition of any one of claims 53-54, wherein the structurally altered variant is a structurally altered variant of the first respiratory virus antigenic polypeptide.

56. The multivalent RNA composition of any one of claims 53-55, wherein the structurally altered variant is a structurally altered variant of the second respiratory virus antigenic polypeptide.

57. A multivalent RNA composition, comprising 5 to 15 messenger ribonucleic acid (mRNA) polynucleotides, each comprising an open reading frame (ORF) encoding a distinct respiratory virus antigenic polypeptide, wherein the respiratory virus antigenic polypeptides are derived from two different viral families, wherein the two viral families comprise influenza viruses and coronaviruses; and a lipid nanoparticle.

58. The multivalent RNA composition of claim 57, wherein the composition has 3-6 mRNA polynucleotides comprising an ORF encoding an influenza antigen.

59. The multivalent RNA composition of any one of claim 57-58, wherein the composition has 1-5 mRNA polynucleotides comprising an ORF encoding a coronavirus antigen.

60. A multivalent RNA composition, comprising a set of at least 6 messenger ribonucleic acid (mRNA) polynucleotides, each comprising an open reading frame (ORF) encoding a respiratory virus antigenic polypeptide from a first or second virus; wherein the first virus is an influenza virus, wherein the second virus is a coronavirus, and wherein the composition comprises a ratio of mRNA polynucleotides encoding respiratory virus antigenic polypeptides of 4:1, 4:2, or 4:3 from the first virus to the second virus.

61. The multivalent RNA composition of any one of claims 40-60, wherein the first and second mRNA polynucleotides are present in the combination vaccine in a ratio of 1:1.

62. The multivalent RNA composition of any one of claims 40-60, wherein the multivalent RNA composition comprises a ratio of mRNA polynucleotides encoding respiratory virus antigenic polypeptides of 4:1 from the first virus to the second virus.

63. The multivalent RNA composition of any one of claims 40-60, wherein the multivalent RNA composition comprises a ratio of mRNA polynucleotides encoding respiratory virus antigenic polypeptides of 3:1 from the first virus to the second virus.

64. The multivalent RNA composition of any one of claims 40-60, wherein the multivalent RNA composition comprises a ratio of mRNA polynucleotides encoding respiratory virus antigenic polypeptides of 2:1 from the first virus to the second virus.

65. The multivalent RNA composition of any one of claims 40-60, wherein the multivalent RNA composition comprises a ratio of mRNA polynucleotides encoding respiratory virus antigenic polypeptides of 5:1 from the first virus to the second virus.

66. The multivalent RNA composition of any one of claims 40-60, wherein the multivalent RNA composition comprises a ratio of mRNA polynucleotides encoding respiratory virus antigenic polypeptides of 4:2 from the first virus to the second virus.

67. The multivalent RNA composition of any one of claims 40-60, wherein the multivalent RNA composition comprises a ratio of mRNA polynucleotides encoding respiratory virus antigenic polypeptides of 1:2 from the first virus to the second virus.

68. The multivalent RNA composition of any one of claims 40-60, wherein the multivalent RNA composition comprises a ratio of mRNA polynucleotides encoding respiratory virus antigenic polypeptides of 8:1 or 8:2 from the first virus to the second virus.

69. The multivalent RNA composition of any one of claims 40-68, wherein the antigenic polypeptides include a Fusion (F) protein, a spike (S) protein, and a hemagglutinin antigen (HA).

70. The multivalent RNA composition of claim 69, further comprising a neuraminidase (NA) antigen.

71. The multivalent RNA composition of any one of claims 40-70, further comprising at least one lipid nanoparticle (LNP).

72. The multivalent RNA composition 71, wherein the LNP comprises a molar ratio of 20-60% ionizable amino lipid, 5-25% non-cationic lipid, 25-55% sterol, and 0.5-15% PEG-modified lipid.

73. The multivalent RNA composition of claim 72, wherein the ionizable amino lipid comprises the structure of Compound 1:

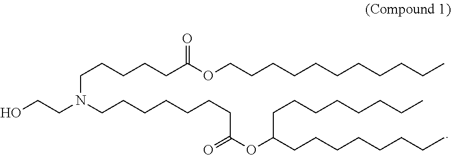 [SMILES: CCCCCCCCCCCOC(=O)CCCCCN(CCO)CCCCCCCC(=O)OC(CCCCCCCC)CCCCCCCC]

74. The combination vaccine of any one of claims 1-39 or the multivalent RNA composition of any one of claims 40-73, wherein the respiratory virus antigenic polypeptide comprises a cell surface glycoprotein.

75. A method for vaccinating a subject, comprising:
administering to the subject a combination vaccine, wherein the combination vaccine comprises a first messenger ribonucleic acid (mRNA) polynucleotide comprising an open reading frame (ORF) encoding a first respiratory virus antigenic polypeptide from an influenza virus; and a second mRNA polynucleotide comprising an ORF encoding a second respiratory virus antigenic polypeptide from a coronavirus.

76. The method of claim 75, wherein the subject is 65 years of age or older.

77. The method of claim 75, wherein the subject is under 18 years of age.

78. The method of claim 75, wherein the method prevents a respiratory infection in the subject.

79. The method of claim 75, wherein the method reduces the severity of a respiratory infection in the subject.

80. The method of claim 75, wherein the subject is seronegative for at least one of the antigenic polypeptides.

81. The method of claim 75, wherein the subject is seronegative for all of the antigenic polypeptides.

82. The method of claim 75, wherein the subject is seropositive for at least one of the antigenic polypeptides.

83. The method of claim 75, wherein the subject is seropositive for all of the antigenic polypeptides.

84. The method of any one of claims 75-83, further comprising administering a booster vaccine.

85. The method of claim 84, wherein the booster vaccine is administered between 3 weeks and 1 year after the combination vaccine.

86. The method of claim 84 or 85, wherein the booster vaccine comprises at least one mRNA polynucleotide comprising an ORF encoding the first or second respiratory virus antigenic polypeptides.

87. The method of claim 84 or 85, wherein the booster vaccine comprises at least one mRNA polynucleotide comprising an ORF encoding the first and second respiratory virus antigenic polypeptides.

88. The method of claim 84 or 85, wherein the booster vaccine comprises at least one mRNA polynucleotide comprising an ORF encoding a structurally altered variant of the first or second respiratory virus antigenic polypeptides.

89. The method of any one of claims 84-88, wherein the combination vaccine is a seasonal booster vaccine.

90. The method of any one of claims 75-89, wherein the combination vaccine is a vaccine of any one of claims 1-74.

91. A method of preventing or reducing the severity of a respiratory infection by administering the combination/multivalent vaccine of any one of claims 1-74 to a subject in an effective amount to prevent infection or reduce the severity of a respiratory infection in the subject based on a single dose or single dose with a booster.

92. The method of any one of claims 75-91, wherein the combination vaccine is administered to the subject in a dose of 25 μg, 50 μg, or 100 μg.

93. The method of any one of claims 75-92, wherein each RNA polynucleotide of the vaccine is formulated in a separate LNP.

94. The method of any one of claims 75-93, wherein the RNA polynucleotides of the vaccine are co-formulated in an LNP.

95. The combination vaccine of any one of claims 1-39 or the multivalent RNA composition of any one of claims 40-74, comprising mRNA polynucleotides encoding four HA antigens.

96. The combination vaccine or multivalent RNA composition of claim 95, wherein the four HA antigens are present in a 1:1:1:1 ratio.

97. The combination vaccine or multivalent RNA composition of claim 95 or 96, further comprising mRNA polynucleotides encoding four NA antigens.

98. The combination vaccine or the multivalent RNA composition of claim 97, wherein the four NA antigens are present in a 1:1:1:1 ratio.

99. The combination vaccine or the multivalent RNA composition of claim 98, wherein the ratio of HA antigens to NA antigens is 1:1.

100. The combination vaccine or the multivalent RNA composition of claim 98, wherein the ratio of HA antigens to NA antigens is 3:1.

101. The method of any one of claims 75-94, wherein the combination vaccine comprises mRNA polynucleotides encoding four HA antigens.

102. The method of claim 101, wherein the four HA antigens are present in a 1:1:1:1 ratio.

103. The method of claim 101 or 102, further comprising mRNA polynucleotides encoding four NA antigens.

104. The method of claim 103, wherein the four NA antigens are present in a 1:1:1:1 ratio.

105. The method of claim 104, wherein the ratio of HA antigens to NA antigens is 1:1.

106. The method of claim 104, wherein the ratio of HA antigens to NA antigens is 3:1.

107. The combination vaccine of any one of claims 1-39, the multivalent RNA composition of any one of claims 40-74 and 95-100, and the method of any one of claims 75-94 and 101-106, wherein the coronavirus is a betacoronavirus.


SEQUENCE LISTING (68 sequences)

SEQ ID NO: 1 - RNA, 3819 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 2 - RNA, 3810 nt, Artificial sequence
  Synthetic
auguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccagggcgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcguggagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 3 - RNA, 1572 nt, Artificial sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 4 - RNA, 1563 nt, Artificial sequence
  Synthetic
auguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 5 - RNA, 1698 nt, Artificial sequence
  Synthetic
augaagaccaucaucgcccugagcuacauccugugccugggcuucacccagaagauccccggcaacgauaacagcaccgccacccugugucugggacaccacgccgugcccaacggcaccaucgugaagacuaucaccaacgaccggaucgaggugaccaacgccaccgagcuggugcagaacagcagcaucggcgagaucugcgacagcccucaccagauccuggacggcggcaacugcacccugaucgacgcacugcugggcgacccucagugcgacggcuuucagaacaagaagugggaccuguucguggagagaucgcgggccuacagcaacugcuaccccuacgacguccccgacuacgcaagccugagaagccucguggccucaagcggcacccuggaguucaagaacgagagcuucaacugggccggcgugacccagaacggcaagucauucagcugcauccggggcuccagcagcagcuucuucucacggcugaacuggcugacccaccugaacuacaccuaccccgcccugaacgugaccaugcccaacaaggagcaguucgacaagcuguacaucuggggagugcaccaucccggcaccgacaaggaccagauuagccuguacgcccagucuagcggccggaucaccgugagcaccaagcggagccagcaggccgugauccccaacaucggcucucggcccagaauccgggacauccccagccggaucagcaucuacuggaccauugugaagcccggcgacauccugcugaucaacuccaccggcaaccugaucgccccucggggcuauuucaagauccggagcggcaagagcagcaucaugcggagcgacgccccuaucggcaagugcaagagcgagugcaucacacccaacggaagcauccccaacgacaagcccuuccagaacgugaaccggauaaccuacggcgccugcccuagauacgugaagcagaacacccugaagcuggccaccggcaugcggaacgugcccgagaagcagacucggggcaucuucggcgccaucgccggcuucaucgagaacggcugggagggcaugguggacggcugguacggcuuccggcaccagaacucugagggcagaggacaggccgcagaccugaagagcacccaggccgccaucgaccagaucaacggcaagcugaaccggcugaucggcaagaccaacgagaaguuccaccagaucgagaaggaguucagcgagguggagggcaggguacaggaccuggagaaguacguggaggacaccaagaucgaccuguggagcuacaacgccgagcugcugguagcccuggagaaccagcacaccaucgaccugaccgacagcgagaugaacaagcuguucgagaagaccaagaagcagcugcgggagaacgccgaggacaugggcaacggcugcuucaagaucuaccacaagugcgacaacgccugcaucggcagcauccggaacgagaccuacgaccacaacguguaccgggacgaggcccugaacaaccgguuccagaucaagggcguggagcugaagagcggcuacaaggacuggauccuguggaucagcuucgccaucuccugcuuccugcugugcguggcccugcuggguuucaucaugugggccugccagaagggcaacauccggugcaacaucugcauc

SEQ ID NO: 6 - RNA, 1745 nt, Artificial sequence
  Synthetic
augaaggccaucaucgugcuguuaaugguggugaccagcaacgccgaccggaucugcaccggcaucaccucuagcaacagcccucacguggugaagaccgccacacagggcgaggugaacgugaccggcgugauuccccugaccaccaccccuaccaagagccacuucgccaaccugaagggaaccgagacccggggcaagcugugucccaagugccugaacugcaccgaccuggacguggcccugggcagacccaagugcaccggcaagauccccagcgcccgggugucuauccugcacgaagugcggcccgugacuagcggcugcuuccccaucaugcacgaccggaccaagauccggcagcugcccaaccugcugcggggcuacgagcacgugcggcugagcacccacaacgugaucaacgccgaagacgcacccgggagaccauacgagaucggcaccagcggcucuugccccaacaucaccaacggcaacggcuucuucgcuaccauggccugggccgugccaaagaacaagacugccaccaacccucugaccaucgaggugcccuacaucugcaccgagggcgaggaccagaucaccguguggggcuuccacagcgacagcgagacccagauggccaagcuguacggcgacagcaagccccagaaguucaccagcagcgccaacggcgugaccacccacuacgugagccagaucggcggcuuccccaaccagaccgaggacggcggcuuaccccagagcggccggaucgugguggacuacauggugcagaagagcggcaagaccggcaccaucaccuaccagcggggcauccugcugccacagaagguguggugcgccucagggcggucaaaggugaucaagggcagccugccacugauuggcgaggccgacugccugcacgagaaguacggcggccugaacaagagcaagcccuacuacaccggcgagcacgccaaggcaaucggcaacugccccaucugggugaagacaccccugaagcuggccaacggcaccaaguaccggccacccgccaaacugcugaaggagcggggcuucuucggcgccauugccggcuuccucgaaggcgguugggagggcaugaucgccggcuggcacggcuacacuagccacggcgcacacggaguagcaguggccgccgaccugaagagcacccaggaggccaucaacaagaucaccaagaaccugaacagccugagcgagcuggaggugaagaaucugcagcggcugucuggcgcuauggacgagcugcacaacgagauccuggagcuggacgagaagguggacgacuuacgggccgacaccaucagcagccagaucgagcuggccgugcugcugagcaacgagggcaucaucaacagcgaggacgagcaccugcuggcccuggagggaagcugaagaagaugcugggcccuucugccguggagaucgguaacggcugcuucgagaccaagcacaagugcaaccagaccugccuggaucggaucgcagccggcaccuuugacgccggggaguucagccugcccaccuucgacagccugaacaucaccgccgccagccugaacgacgacggccuggacaaccacaccauccugcuguacuacucuacagccgcuagcagccuggccgugacccugaugaucgccaucuucgugguguacauggugagccgggacaacgugagcugcagcaucugccug

SEQ ID NO: 7 - RNA, 1752 nt, Artificial sequence
  Synthetic
augaaggccaucaucgugcuacugaugguggugaccagcaacgccgaccggaucugcaccggcaucaccagcagcaacagcccgcacguggugaagaccgccacccaaggcgaggugaacgugaccggcgugaucccacugaccaccacucccaccaagagcuacuucgccaaccugaagggcacacggacucggggcaagcugugccccgacugccugaacugcaccgaccuggacguggcccugggcagacccaugugcgugggcaccaccccuucugccaaggccagcauccugcacgaggugagacccgugaccagcgggugcuuccccaucaugcacgaccggaccaagauccggcagcugcccaaccugcugcggggcuacgagaagauccggcugagcacccagaacgugaucgacgccgagaaggccccuggaggucccuaccggcugggcaccagcggaagcugccccaacgccaccagcaagaucggcuucuucgccaccauggccugggcugugcccaaggacaacuacaagaacgccaccaauccccugaccguggaggugcccuacaucugcaccgagggcgaggaccagaucaccguguggggcuuccacagcgacaacaagacccagaugaagagccuguacggcgacagcaauccccagaaguucacaagcagcgccaacggcgugaccacccacuacgugagccagaucggcgacuuccccgaccagaccgaggacggagggcugccucagaguggccggaucgugguggacuacaugaugcagaagcccggcaagaccggcaccaucguguaccagcggggcgugcuguugccucagaaaguuuggugugccagcggcaggagcaaggugaucaagggcagccugccccugaucggcgaggcagacugccuccacgaggaguacggcggccugaacaagagcaagcccuacuacaccggcaagcacgccaaggccaucggcaacugccccaucugggugaagaccccucugaagcuggccaacggcaccaaguaccggccaccagccaagcugcugaaggagcggggcuucuuuggcgccauugccggcuuccucgagggaggcugggagggcaugaucgccggcuggcacggcuacacaagccacggcgcacacggaguggcuguggcugccgaccugaagagcacccaggaggccaucaacaagaucaccaagaaccugaacagccugagcgagcuggaggugaagaaccugcagcggcugucaggcgccauggacgagcugcacaacgagauccuggagcuggacgagaagguggacgaccugcgugccgacaccaucagcagccagaucgagcuggccgugcugcugagcaacgagggcaucaucaacagcgaggacgagcaccugcuggcccuggagcggaaacugaagaagaugcugggacccucugccguggacaucggcaacggcugcuucgagaccaagcacaagugcaaccagaccugccuggaucggaucgccgccggaaccuucaacgccggcgaguucagccugcccaccuucgacagccugaacaucaccgccgccagccugaacgacgacggccuggacaaccacaccauccugcuguacuacagcacugccgccucaagccuggccgugacccugaugcuggccaucuucaucguguacauggugagccgggacaacgugagcugcagcaucugccug

SEQ ID NO: 8 - RNA, 1698 nt, Artificial sequence
  Synthetic
augaaggccauccuggucgugaugcuguacaccuucaccaccgccaacgccgacacccugugcaucggcuaccacgccaacaacagcaccgacaccguggacaccgugcuggagaagaacgugaccgugacccacagcgugaaccugcuggaggacaagcacaacggcaagcugugcaagcugaggggaguggcaccccugcaccugggcaagugcaacaucgccggcuggauccugggcaaccccgagugcgagagccugagcacagcccggagcuggagcuacaucguggagaccagcaacagcgacaacggcaccuguuaccccggcgacuucaucaacuacgaggagcugcgggagcagcugagcagcgugagcagcuucgagcgguucgagaucuuccccaagaccagcagcuggcccaaccacgacagcgacaacggcgugacagcagccuguccacacgccggagccaagagcuucuacaagaaccugaucuggcuggugaagaagggcaagagcuaccccaagaucaaccagaccuacaucaacgacaagggcaaggaggugcuggugcuguggggcauccaccacccaccuaccaucgccgaccagcagagccuguaccagaacgccgacgccuacguguucgugggcaccagccgguacagcaagaaguucaagccagagaucgccacccggcccaaggugagagaccaggagggccggaugaacuacuacuggacccugguggagcccggagacaagauuaccuucgaggccaccggcaaccugguggccccucgguacgccuucaccauggaacgggacgcuggcagcggcaucaucaucagcgacacucccgugcacgacugcaacaccaccugccagacucccgagggcgcuaucaacaccagccugcccuuccagaacgugcaccccaucaccaucggcaagugccccaaguacguaaagagcaccaaauugcggcuggccaccggacucaggaacgugcccagcauccaaagccggggccuguuuggcgcaaucgccggcuucaucgagggcggcuggacuggcaugguggacggcugguacggcuaccaccaccagaacgaacaggggagcggcuacgcagcugaccugaagagcacccagaacgccaucgacaagaucaccaacaaggugaacagcgugaucgagaagaugaacacccaguucaccgccgugggcaaggaguucaaccaccuggagaagcggaucgagaaccugaacaagaagguggacgacggcuuccuggacaucuggaccuacaacgccgagcugcugguucugcuggagaacgagcggacccuggacuaucacgacagcaacgugaagaaccuguacgagaaggugcggaaccagcugaagaacaacgccaaggagaucggcaacggcugcuucgaguucuaccacaagugcgacaacaccugcauggagagcgugaagaacggcaccuacgacuaccccaaguacagcgaggaggccaagcugaaccgggagaagaucgacggcgugaagcuggacagcacccggaucuaccagauccuggccaucuacagcaccguggccagcagccuggugcugguggugagccugggcgccaucagcuucuggaugugcagcaacggcagccugcagugccggaucugcauc

SEQ ID NO: 9 - RNA, 1407 nt, Artificial sequence
  Synthetic
augaacccgaaccagaagaucaucaccaucggcagcgugagccugaccaucagcaccaucugcuucuucaugcagaucgccauccugaucaccaccgugacccugcacuucaagcaguacgaguucaacagccugcccaacaaccaggugaugcugugcgagcccaccaucaucgagcggaacaucaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcgggaccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggagagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagcuacgugugcagcggucuggugggcgacacuccccggaagagcgacagcagcuccagcagccacugccugaaccccaacaacgaggaggguggucacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccgaucugaaccugaugcacauc

SEQ ID NO: 10 - RNA, 1398 nt, Artificial sequence
  Synthetic
augcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccggggugaucggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggagagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccug

SEQ ID NO: 11 - RNA, 1407 nt, Artificial sequence
  Synthetic
augaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaaggaccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggagagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaag

SEQ ID NO: 12 - RNA, 1398 nt, Artificial sequence
  Synthetic
augcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagaggaccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggagagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccug

SEQ ID NO: 13 - RNA, 1407 nt, Artificial sequence
  Synthetic
augaacccgaaccagaagaucaucaccaucggcagcgugagccugaccaucagcaccaucugcuucuucaugcagaucgccauccugaucaccaccgugacccugcacuucaagcaguacgaguucaacagcccucccaacaaccaggugaugcugugcgagcccaccaucaucgagcggaacaugaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcggggccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggagagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagauacgugugcagcggucuggugggcgacacuccccggaagagcgacagcagcuccagcagccacugccugaaccccaacaacgagaagggugaccacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccaaccugagccugaugcacauc

SEQ ID NO: 14 - RNA, 1407 nt, Artificial sequence
  Synthetic
augaacccgaaccagaagaucaucaccaucggcagcgugagccugaccaucagcaccaucugcuucuucaugcagaucgccauccugaucaccaccgugacccugcacuucaagcaguacgaguucaacagcccucccaacaaccaggugaugcugugcgagcccaccaucaucgagcggaacaugaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcgggaccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggacagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagauacgugugcagcggucuggugggcgacacuccccggaagagcgacagcagcuccagcagccacugccugaaccccaacaacgagaagggugaccacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccaaccugagccugaugcacauc

SEQ ID NO: 15 - protein, 1273 aa, Artificial sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1270 aa, Artificial sequence
  Synthetic
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 524 aa, Artificial sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISF

SEQ ID NO: 18 - protein, 521 aa, Artificial sequence
  Synthetic
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKSGGGSILAIYSTVASSLVLLVSLGAISF

SEQ ID NO: 19 - protein, 566 aa, Influenza virus
MKTIIALSYILCLGFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 20 - protein, 582 aa, Influenza virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDSETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 21 - protein, 584 aa, Influenza virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 22 - protein, 566 aa, Influenza virus
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 23 - protein, 469 aa, Influenza virus
MNPNQKIITIGSVSLTISTICFFMQIAILITTVTLHFKQYEFNSLPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 24 - protein, 466 aa, Influenza virus
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALTFSGVMVSMKEPGWYSFGFEIKDKKCDVPCIGIEMVHDGGKETWHSAATAIYCLMGSGQLLWDTVTGVDMAL

SEQ ID NO: 25 - protein, 469 aa, Influenza virus
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 26 - protein, 466 aa, Influenza virus
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 27 - protein, 469 aa, Artificial sequence
  Synthetic
MNPNQKIITIGSVSLTISTICFFMQIAILITTVTLHFKQYEFNSPPNNQVMLCEPTIIERNMTEIVYLTNTTIEKEICPKPAEYRNWSKPQCGITGFAPFSKDNSIRLSAGGDIWVTREPYVSCDLDKCYQFALGQGTTLNNVHSNNTVRGRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 28 - protein, 469 aa, Artificial sequence
  Synthetic
MNPNQKIITIGSVSLTISTICFFMQIAILITTVTLHFKQYEFNSPPNNQVMLCEPTIIERNMTEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQDSECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 29 - RNA, 47 nt, Artificial sequence
  Synthetic
gggaaauaagagagaaaagaagaguaagaagaaauauaagagccacc

SEQ ID NO: 30 - RNA, 57 nt, Artificial sequence
  Synthetic
gggaaauaagagagaaaagaagaguaagaagaaauauaagaccccggcgccgccacc

SEQ ID NO: 31 - RNA, 119 nt, Artificial sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 32 - RNA, 119 nt, Artificial sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 33 - RNA, 3995 nt, Artificial sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 34 - RNA, 1748 nt, Artificial sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 35 - RNA, 1739 nt, Artificial sequence
  Synthetic
gggaaauaagagagaaaagaagaguaagaagaaauauaagaccccggcgccgccaccauguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 36 - RNA, 3986 nt, Artificial sequence
  Synthetic
gggaaauaagagagaaaagaagaguaagaagaaauauaagaccccggcgccgccaccauguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccagggcgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcguggagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 37 - RNA, 1874 nt, Artificial sequence
  Synthetic
gggaaauaagagagaaaagaagaguaagaagaaauauaagaccccggcgccgccaccaugaagaccaucaucgcccugagcuacauccugugccugggcuucacccagaagauccccggcaacgauaacagcaccgccacccugugucugggacaccacgccgugcccaacggcaccaucgugaagacuaucaccaacgaccggaucgaggugaccaacgccaccgagcuggugcagaacagcagcaucggcgagaucugcgacagcccucaccagauccuggacggcggcaacugcacccugaucgacgcacugcugggcgacccucagugcgacggcuuucagaacaagaagugggaccuguucguggagagaucgcgggccuacagcaacugcuaccccuacgacguccccgacuacgcaagccugagaagccucguggccucaagcggcacccuggaguucaagaacgagagcuucaacugggccggcgugacccagaacggcaagucauucagcugcauccggggcuccagcagcagcuucuucucacggcugaacuggcugacccaccugaacuacaccuaccccgcccugaacgugaccaugcccaacaaggagcaguucgacaagcuguacaucuggggagugcaccaucccggcaccgacaaggaccagauuagccuguacgcccagucuagcggccggaucaccgugagcaccaagcggagccagcaggccgugauccccaacaucggcucucggcccagaauccgggacauccccagccggaucagcaucuacuggaccauugugaagcccggcgacauccugcugaucaacuccaccggcaaccugaucgccccucggggcuauuucaagauccggagcggcaagagcagcaucaugcggagcgacgccccuaucggcaagugcaagagcgagugcaucacacccaacggaagcauccccaacgacaagcccuuccagaacgugaaccggauaaccuacggcgccugcccuagauacgugaagcagaacacccugaagcuggccaccggcaugcggaacgugcccgagaagcagacucggggcaucuucggcgccaucgccggcuucaucgagaacggcugggagggcaugguggacggcugguacggcuuccggcaccagaacucugagggcagaggacaggccgcagaccugaagagcacccaggccgccaucgaccagaucaacggcaagcugaaccggcugaucggcaagaccaacgagaaguuccaccagaucgagaaggaguucagcgagguggagggcaggguacaggaccuggagaaguacguggaggacaccaagaucgaccuguggagcuacaacgccgagcugcugguagcccuggagaaccagcacaccaucgaccugaccgacagcgagaugaacaagcuguucgagaagaccaagaagcagcugcgggagaacgccgaggacaugggcaacggcugcuucaagaucuaccacaagugcgacaacgccugcaucggcagcauccggaacgagaccuacgaccacaacguguaccgggacgaggcccugaacaaccgguuccagaucaagggcguggagcugaagagcggcuacaaggacuggauccuguggaucagcuucgccaucuccugcuuccugcugugcguggcccugcuggguuucaucaugugggccugccagaagggcaacauccggugcaacaucugcaucugauaauaggcuggagccucgguggccuagcuucuugccccuugggccuccccccagccccuccuccccuuccugcacccguacccccguggucuuugaauaaagucugagugggcggc

SEQ ID NO: 38 - RNA, 1921 nt, Artificial sequence
  Synthetic
gggaaauaagagagaaaagaagaguaagaagaaauauaagaccccggcgccgccaccaugaaggccaucaucgugcuguuaaugguggugaccagcaacgccgaccggaucugcaccggcaucaccucuagcaacagcccucacguggugaagaccgccacacagggcgaggugaacgugaccggcgugauuccccugaccaccaccccuaccaagagccacuucgccaaccugaagggaaccgagacccggggcaagcugugucccaagugccugaacugcaccgaccuggacguggcccugggcagacccaagugcaccggcaagauccccagcgcccgggugucuauccugcacgaagugcggcccgugacuagcggcugcuuccccaucaugcacgaccggaccaagauccggcagcugcccaaccugcugcggggcuacgagcacgugcggcugagcacccacaacgugaucaacgccgaagacgcacccgggagaccauacgagaucggcaccagcggcucuugccccaacaucaccaacggcaacggcuucuucgcuaccauggccugggccgugccaaagaacaagacugccaccaacccucugaccaucgaggugcccuacaucugcaccgagggcgaggaccagaucaccguguggggcuuccacagcgacagcgagacccagauggccaagcuguacggcgacagcaagccccagaaguucaccagcagcgccaacggcgugaccacccacuacgugagccagaucggcggcuuccccaaccagaccgaggacggcggcuuaccccagagcggccggaucgugguggacuacauggugcagaagagcggcaagaccggcaccaucaccuaccagcggggcauccugcugccacagaagguguggugcgccucagggcggucaaaggugaucaagggcagccugccacugauuggcgaggccgacugccugcacgagaaguacggcggccugaacaagagcaagcccuacuacaccggcgagcacgccaaggcaaucggcaacugccccaucugggugaagacaccccugaagcuggccaacggcaccaaguaccggccacccgccaaacugcugaaggagcggggcuucuucggcgccauugccggcuuccucgaaggcgguugggagggcaugaucgccggcuggcacggcuacacuagccacggcgcacacggaguagcaguggccgccgaccugaagagcacccaggaggccaucaacaagaucaccaagaaccugaacagccugagcgagcuggaggugaagaaucugcagcggcugucuggcgcuauggacgagcugcacaacgagauccuggagcuggacgagaagguggacgacuuacgggccgacaccaucagcagccagaucgagcuggccgugcugcugagcaacgagggcaucaucaacagcgaggacgagcaccugcuggcccuggagggaagcugaagaagaugcugggcccuucugccguggagaucgguaacggcugcuucgagaccaagcacaagugcaaccagaccugccuggaucggaucgcagccggcaccuuugacgccggggaguucagccugcccaccuucgacagccugaacaucaccgccgccagccugaacgacgacggccuggacaaccacaccauccugcuguacuacucuacagccgcuagcagccuggccgugacccugaugaucgccaucuucgugguguacauggugagccgggacaacgugagcugcagcaucugccugugauaauaggcuggagccucgguggccuagcuucuugccccuugggccuccccccagccccuccuccccuuccugcacccguacccccguggucuuugaauaaagucugagugggcggc

SEQ ID NO: 39 - RNA, 1928 nt, Artificial sequence
  Synthetic
gggaaauaagagagaaaagaagaguaagaagaaauauaagaccccggcgccgccaccaugaaggccaucaucgugcuacugaugguggugaccagcaacgccgaccggaucugcaccggcaucaccagcagcaacagcccgcacguggugaagaccgccacccaaggcgaggugaacgugaccggcgugaucccacugaccaccacucccaccaagagcuacuucgccaaccugaagggcacacggacucggggcaagcugugccccgacugccugaacugcaccgaccuggacguggcccugggcagacccaugugcgugggcaccaccccuucugccaaggccagcauccugcacgaggugagacccgugaccagcgggugcuuccccaucaugcacgaccggaccaagauccggcagcugcccaaccugcugcggggcuacgagaagauccggcugagcacccagaacgugaucgacgccgagaaggccccuggaggucccuaccggcugggcaccagcggaagcugccccaacgccaccagcaagaucggcuucuucgccaccauggccugggcugugcccaaggacaacuacaagaacgccaccaauccccugaccguggaggugcccuacaucugcaccgagggcgaggaccagaucaccguguggggcuuccacagcgacaacaagacccagaugaagagccuguacggcgacagcaauccccagaaguucacaagcagcgccaacggcgugaccacccacuacgugagccagaucggcgacuuccccgaccagaccgaggacggagggcugccucagaguggccggaucgugguggacuacaugaugcagaagcccggcaagaccggcaccaucguguaccagcggggcgugcuguugccucagaaaguuuggugugccagcggcaggagcaaggugaucaagggcagccugccccugaucggcgaggcagacugccuccacgaggaguacggcggccugaacaagagcaagcccuacuacaccggcaagcacgccaaggccaucggcaacugccccaucugggugaagaccccucugaagcuggccaacggcaccaaguaccggccaccagccaagcugcugaaggagcggggcuucuuuggcgccauugccggcuuccucgagggaggcugggagggcaugaucgccggcuggcacggcuacacaagccacggcgcacacggaguggcuguggcugccgaccugaagagcacccaggaggccaucaacaagaucaccaagaaccugaacagccugagcgagcuggaggugaagaaccugcagcggcugucaggcgccauggacgagcugcacaacgagauccuggagcuggacgagaagguggacgaccugcgugccgacaccaucagcagccagaucgagcuggccgugcugcugagcaacgagggcaucaucaacagcgaggacgagcaccugcuggcccuggagcggaaacugaagaagaugcugggacccucugccguggacaucggcaacggcugcuucgagaccaagcacaagugcaaccagaccugccuggaucggaucgccgccggaaccuucaacgccggcgaguucagccugcccaccuucgacagccugaacaucaccgccgccagccugaacgacgacggccuggacaaccacaccauccugcuguacuacagcacugccgccucaagccuggccgugacccugaugcuggccaucuucaucguguacauggugagccgggacaacgugagcugcagcaucugccugugauaauaggcuggagccucgguggccuagcuucuugccccuugggccuccccccagccccuccuccccuuccugcacccguacccccguggucuuugaauaaagucugagugggcggc

SEQ ID NO: 40 - RNA, 1874 nt, Artificial sequence
  Synthetic
gggaaauaagagagaaaagaagaguaagaagaaauauaagaccccggcgccgccaccaugaaggccauccuggucgugaugcuguacaccuucaccaccgccaacgccgacacccugugcaucggcuaccacgccaacaacagcaccgacaccguggacaccgugcuggagaagaacgugaccgugacccacagcgugaaccugcuggaggacaagcacaacggcaagcugugcaagcugaggggaguggcaccccugcaccugggcaagugcaacaucgccggcuggauccugggcaaccccgagugcgagagccugagcacagcccggagcuggagcuacaucguggagaccagcaacagcgacaacggcaccuguuaccccggcgacuucaucaacuacgaggagcugcgggagcagcugagcagcgugagcagcuucgagcgguucgagaucuuccccaagaccagcagcuggcccaaccacgacagcgacaacggcgugacagcagccuguccacacgccggagccaagagcuucuacaagaaccugaucuggcuggugaagaagggcaagagcuaccccaagaucaaccagaccuacaucaacgacaagggcaaggaggugcuggugcuguggggcauccaccacccaccuaccaucgccgaccagcagagccuguaccagaacgccgacgccuacguguucgugggcaccagccgguacagcaagaaguucaagccagagaucgccacccggcccaaggugagagaccaggagggccggaugaacuacuacuggacccugguggagcccggagacaagauuaccuucgaggccaccggcaaccugguggccccucgguacgccuucaccauggaacgggacgcuggcagcggcaucaucaucagcgacacucccgugcacgacugcaacaccaccugccagacucccgagggcgcuaucaacaccagccugcccuuccagaacgugcaccccaucaccaucggcaagugccccaaguacguaaagagcaccaaauugcggcuggccaccggacucaggaacgugcccagcauccaaagccggggccuguuuggcgcaaucgccggcuucaucgagggcggcuggacuggcaugguggacggcugguacggcuaccaccaccagaacgaacaggggagcggcuacgcagcugaccugaagagcacccagaacgccaucgacaagaucaccaacaaggugaacagcgugaucgagaagaugaacacccaguucaccgccgugggcaaggaguucaaccaccuggagaagcggaucgagaaccugaacaagaagguggacgacggcuuccuggacaucuggaccuacaacgccgagcugcugguucugcuggagaacgagcggacccuggacuaucacgacagcaacgugaagaaccuguacgagaaggugcggaaccagcugaagaacaacgccaaggagaucggcaacggcugcuucgaguucuaccacaagugcgacaacaccugcauggagagcgugaagaacggcaccuacgacuaccccaaguacagcgaggaggccaagcugaaccgggagaagaucgacggcgugaagcuggacagcacccggaucuaccagauccuggccaucuacagcaccguggccagcagccuggugcugguggugagccugggcgccaucagcuucuggaugugcagcaacggcagccugcagugccggaucugcaucugauaauaggcuggagccucgguggccuagcuucuugccccuugggccuccccccagccccuccuccccuuccugcacccguacccccguggucuuugaauaaagucugagugggcggc

SEQ ID NO: 41 - RNA, 1583 nt, Artificial sequence
  Synthetic
gggaaauaagagagaaaagaagaguaagaagaaauauaagaccccggcgccgccaccaugaacccgaaccagaagaucaucaccaucggcagcgugagccugaccaucagcaccaucugcuucuucaugcagaucgccauccugaucaccaccgugacccugcacuucaagcaguacgaguucaacagccugcccaacaaccaggugaugcugugcgagcccaccaucaucgagcggaacaucaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcgggaccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggagagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagcuacgugugcagcggucuggugggcgacacuccccggaagagcgacagcagcuccagcagccacugccugaaccccaacaacgaggaggguggucacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccgaucugaaccugaugcacaucugauaauaggcuggagccucgguggccuagcuucuugccccuugggccuccccccagccccuccuccccuuccugcacccguacccccguggucuuugaauaaagucugagugggcggc

SEQ ID NO: 42 - RNA, 1574 nt, Artificial sequence
  Synthetic
gggaaauaagagagaaaagaagaguaagaagaaauauaagaccccggcgccgccaccaugcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccggggugaucggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggagagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccugugauaauaggcuggagccucgguggccuagcuucuugccccuugggccuccccccagccccuccuccccuuccugcacccguacccccguggucuuugaauaaagucugagugggcggc

SEQ ID NO: 43 - RNA, 1583 nt, Artificial sequence
  Synthetic
gggaaauaagagagaaaagaagaguaagaagaaauauaagaccccggcgccgccaccaugaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaaggaccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggagagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaagugauaauaggcuggagccucgguggccuagcuucuugccccuugggccuccccccagccccuccuccccuuccugcacccguacccccguggucuuugaauaaagucugagugggcggc

SEQ ID NO: 44 - RNA, 1574 nt, Artificial sequence
  Synthetic
gggaaauaagagagaaaagaagaguaagaagaaauauaagaccccggcgccgccaccaugcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagaggaccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggagagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccugugauaauaggcuggagccucgguggccuagcuucuugccccuugggccuccccccagccccuccuccccuuccugcacccguacccccguggucuuugaauaaagucugagugggcggc

SEQ ID NO: 45 - RNA, 1583 nt, Artificial sequence
  Synthetic
gggaaauaagagagaaaagaagaguaagaagaaauauaagaccccggcgccgccaccaugaacccgaaccagaagaucaucaccaucggcagcgugagccugaccaucagcaccaucugcuucuucaugcagaucgccauccugaucaccaccgugacccugcacuucaagcaguacgaguucaacagcccucccaacaaccaggugaugcugugcgagcccaccaucaucgagcggaacaugaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcggggccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggagagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagauacgugugcagcggucuggugggcgacacuccccggaagagcgacagcagcuccagcagccacugccugaaccccaacaacgagaagggugaccacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccaaccugagccugaugcacaucugauaauaggcuggagccucgguggccuagcuucuugccccuugggccuccccccagccccuccuccccuuccugcacccguacccccguggucuuugaauaaagucugagugggcggc

SEQ ID NO: 46 - RNA, 1583 nt, Artificial sequence
  Synthetic
gggaaauaagagagaaaagaagaguaagaagaaauauaagaccccggcgccgccaccaugaacccgaaccagaagaucaucaccaucggcagcgugagccugaccaucagcaccaucugcuucuucaugcagaucgccauccugaucaccaccgugacccugcacuucaagcaguacgaguucaacagcccucccaacaaccaggugaugcugugcgagcccaccaucaucgagcggaacaugaccgagaucguguaccugaccaacaccaccaucgagaaggagaucugccccaagcccgccgaguaccggaacuggagcaagccccagugcggcaucaccggcuucgccccauucagcaaggacaacagcaucagacugagugccggcggcgacaucugggugacccgggagcccuacgugagcugcgaccuggacaagugcuaccaguucgcccugggacagggcaccacccugaacaacgugcacagcaacaacacugugcgggaccggaccccauaccggacccugcugaugaacgagcugggcgugcccuuccaccugggcaccaagcaggugugcaucgccuggagcagcagcagcugccacgacggcaaggccuggcugcacgugugcauuaccggcgacgacaagaacgccaccgccagcuucaucuacaacggcaggcugguggacagcguggugagcuggagcaacgacauccugcggacccaggacagcgagugcgugugcaucaacggcaccugcaccguggugaugacugacggcaacgccaccggcaaggccgacaccaagauccuguucaucgaggaggggaagaucgugcacaccagcaagcugucuggcagcgcccagcacguggaggagugcagcugcuacccucgguaccccggcgugaggugcgugugccgggacaacuggaagggcagcaaccggcccaucaucgacaucaacaucaaggaccacagcauagugagcagauacgugugcagcggucuggugggcgacacuccccggaagagcgacagcagcuccagcagccacugccugaaccccaacaacgagaagggugaccacggcgugaagggcugggccuucgacgacggcaacgacguguggaugggccggaccaucaacgagaccagcagacugggcuacgagaccuucaaggugguggagggcuggagcaaucccaagagcaagcugcagaucaaccggcaggugaucgucgaucggggcgaucggagcggcuacagcggcaucuucagcguggagggcaagagcugcaucaaccggugcuucuacguggagcugauccggggccggaaggaggagaccgaggugcuguggaccagcaacagcaucgugguguucugcggcaccagcggcaccuacggcaccggauccuggccagacggcgccaaccugagccugaugcacaucugauaauaggcuggagccucgguggccuagcuucuugccccuugggccuccccccagccccuccuccccuuccugcacccguacccccguggucuuugaauaaagucugagugggcggc

SEQ ID NO: 47 - RNA, 1583 nt, Artificial sequence
  Synthetic
gggaaauaagagagaaaagaagaguaagaagaaauauaagaccccggcgccgccaccaugaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaagggccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggagagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaagugauaauaggcuggagccucgguggccuagcuucuugccccuugggccuccccccagccccuccuccccuuccugcacccguacccccguggucuuugaauaaagucugagugggcggc

SEQ ID NO: 48 - RNA, 11 nt, Artificial sequence
  Synthetic
gggauccuacc

SEQ ID NO: 49 - protein, 10 aa, Artificial sequence
  Synthetic
TQTNSPRRAR

SEQ ID NO: 50 - protein, 5 aa, Artificial sequence
  Synthetic
KPSKR

SEQ ID NO: 51 - RNA, 1583 nt, Artificial sequence
  Synthetic
gggaaauaagagagaaaagaagaguaagaagaaauauaagaccccggcgccgccaccaugaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaaggaccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggacagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaagugauaauaggcuggagccucgguggccuagcuucuugccccuugggccuccccccagccccuccuccccuuccugcacccguacccccguggucuuugaauaaagucugagugggcggc

SEQ ID NO: 52 - RNA, 1574 nt, Artificial sequence
  Synthetic
gggaaauaagagagaaaagaagaguaagaagaaauauaagaccccggcgccgccaccaugcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagagggccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggagagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccugugauaauaggcuggagccucgguggccuagcuucuugccccuugggccuccccccagccccuccuccccuuccugcacccguacccccguggucuuugaauaaagucugagugggcggc

SEQ ID NO: 53 - RNA, 1574 nt, Artificial sequence
  Synthetic
gggaaauaagagagaaaagaagaguaagaagaaauauaagaccccggcgccgccaccaugcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagaggaccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggacagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccugugauaauaggcuggagccucgguggccuagcuucuugccccuugggccuccccccagccccuccuccccuuccugcacccguacccccguggucuuugaauaaagucugagugggcggc

SEQ ID NO: 54 - RNA, 1574 nt, Artificial sequence
  Synthetic
gggaaauaagagagaaaagaagaguaagaagaaauauaagaccccggcgccgccaccaugcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccgggguggccggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggagagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccugugauaauaggcuggagccucgguggccuagcuucuugccccuugggccuccccccagccccuccuccccuuccugcacccguacccccguggucuuugaauaaagucugagugggcggc

SEQ ID NO: 55 - RNA, 1574 nt, Artificial sequence
  Synthetic
gggaaauaagagagaaaagaagaguaagaagaaauauaagaccccggcgccgccaccaugcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccggggugaucggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggacagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccugugauaauaggcuggagccucgguggccuagcuucuugccccuugggccuccccccagccccuccuccccuuccugcacccguacccccguggucuuugaauaaagucugagugggcggc

SEQ ID NO: 56 - RNA, 1407 nt, Artificial sequence
  Synthetic
augaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaagggccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggagagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaag

SEQ ID NO: 57 - RNA, 1407 nt, Artificial sequence
  Synthetic
augaaccccaaccagaagaucaucaccaucggcagcaucugcaugaccaucggcaccgccaaccugauccugcaaaucggcaacaucaucagcaucugggugagccacagcauccagaucggcaaccagagccagaucgagaccugcaacaagagcgugaucaccuacgagaacaacaccugggugaaccagaccuucgugaacaucagcaacaccaacagcgccgcucggcagucaguggccagcgugaagcuggccggcaacagcagccugugccccguuaguggcugggccaucuacagcaaggacaacagcgugcggaucggcagcaagggcgacguguucgugauccgggagcccuucaucagcugcagcccgcuugagugccgcaccuucuuccugacccagggcgcucugcugaacgacaagcacagcaacggcaccaucaaggaccggagccccuaucggacccugaugagcugccccauuggcgaggugcccagccccuacaacagccgguucgagucuguggccuggagcgccucugccugccacgacggcaccaacuggcugaccaucgggaucagcggacccgauagcggagcaguggccgugcugaaguacaacggcaucaucaccgacaccaucaagagcuggcggaacaagauccugcggacccaggacagcgagugcgccugcgugaacggcagcugcuucaccaucaugaccgacggcccuagcgacggacaggccagcuacaagaucuuccggaucgagaagggcaagaucaucaagagcguggagaugaaggcacccaacuaccacuacgaggagugcagcugcuaccccgacagcagcgagaucaccugcgugugccgggacaacuggcacgggagcaacaggcccugggugagcuucaaccagaaccuggaguaccagaugggcuacaucugcagcggcguguucggcgacaacccacggcccaacgacaagacuggcagcugcgguccggugagcagcaacggcgccaacggcgugaagggcuucagcuucaaguacggcaacggcguguggaucggccggaccaagagcaucagcagccggaagggcuucgagaugaucugggaccccaacggcuggaccggcaccgacaacaaguucagcaagaagcaggacaucgugggcaucaacgaguggagcggcuacagcggcagcuucgugcagcaccccgagcugacuggccugaacugcauccggcccugcuucuggguggaacugauacggggacggcccgaggagaacaccaucuggaccagcggcagcagcaucagcuucugcggcguggacagcgauaucgugggcuggagcuggccagacggagccgagcugcccuucaccaucgacaag

SEQ ID NO: 58 - RNA, 1398 nt, Artificial sequence
  Synthetic
augcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagagggccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggagagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccug

SEQ ID NO: 59 - RNA, 1398 nt, Artificial sequence
  Synthetic
augcugcccagcaccauccagacccugacccuguuccugaccagcggaggcgugcugcugagccuguacgucagcgccagccugagcuaccugcuguacagcgacauccugcugaaguucagccggaccgaggugaccgcucccaucaugccccuggacugcgccaacgccagcaacgugcaggccgugaaucggagcgccaccaagggcgugacuccccugcugcccgagccugaguggacuuauccucggcugagcugcccaggcagcaccuuccagaaggcccugcugaucagcccacaccgguucggcgagaccaagggcaacagcgcuccccugaucauccgggagcccuucaucgccugcggccccaaggagugcaagcacuucgcccugacccacuacgcugcccaacccggaggcuacuacaacggcaccagagaggaccggaacaagcugcggcaccugaucagcgugaagcugggcaagauccccaccguggagaacagcaucuuccacauggcugcuuggucuggaagugcuugucacgacggccgggaguggaccuacaucggcguggacggcccagacagcaacgcccugcugaagaucaaguacggcgaggccuacaccgacaccuaccacagcuacgccaagaacauccugcggacccaggacagcgccugcaacugcaucggcggcgacugcuaccugaugaucaccgacggcccagcaucuggcaucagcgagugccgguuccugaagauccgggagggccggaucaucaaggagaucuuccccaccgggagagugaagcacaccgaggagugcaccugcggcuucgccagcaacaagaccaucgagugcgccugccgggacaacagcuacaccgccaagcggcccuucgugaagcugaacguggagaccgacaccgccgagauccggcugaugugcaccaagaccuaccuggacaccccucggcccaacgacggaagcaucaccggacccugcgagagcgacggggacgaaggaagcggcggaaucaagggcggcuucgugcaccagcggauggccagcaagaucggccggugguacagccggaccaugagcaagaccaagcggaugggcaugggccuguacgugaaguacgacggcgaccccuggacagacagcgaagcccuggcccugucuggcgugauggugagcauggaggagcccggcugguacagcuucggcuucgagaucaaggacaagaagugcgacgugcccugcaucggcaucgagauggugcacgacggcggcaagaccaccuggcauagcgccgcaaccgcgaucuacugccugaugggcagcggccagcugcugugggacaccgugaccggcgugaacaugacccug

SEQ ID NO: 60 - RNA, 1398 nt, Artificial sequence
  Synthetic
augcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccgggguggccggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggagagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccug

SEQ ID NO: 61 - RNA, 1398 nt, Artificial sequence
  Synthetic
augcugcccagcaccauccagacccugacccuguuucugaccagcggaggcgugcugcugagccuguacgugagcgccagccugagcuaccugcuguacagcgacauccugcugaaguucagccccaccgagaucaccgcacccaccaugccccuggacugcgccaacgccagcaacgugcaggccgugaaccggagcgccacaaagggcgugacccugcugcugcccgagccagaguggacauauccucggcugagcugcccuggcagcaccuuccagaaggcccugcugaucagcccacaccgguucggcgagaccaagggcaacagcgcaccccugaucauccgggagcccuucguggccuguggccccaacgagugcaagcacuucgcccugacacacuacgcugcucagcccgguggcuacuacaacggcacccggggugaucggaacaagcugcggcaccugaucagcgugaagcugggcaagauccccaccguggagaacagcaucuuccacauggccgccuggucaggaagcgccugccacgacggcaaggaguggaccuacaucggcguggacggcccugacaacaacgcccugcugaaggugaaguacggcgaggccuacaccgacaccuaccacagcuacgccaacaacauccugcggacccaggacagcgccugcaacugcaucggcggcaacugcuaccugaugaucaccgacggcagcgcuucuggcgugagcgagugccgguuccugaagauccgggagggccggaucaucaaggagaucuuucccaccggccgggugaagcacaccgaggagugcaccugcggcuucgccagcaacaagaccaucgagugcgccugccgggacaaucgguacaccgccaagcggcccuucgugaagcugaacguggagaccgacaccgccgagauccggcugaugugcaccgacacuuaucuggacaccccucggccuaacgacggcagcaucaccggcccuugcgagagcgacggcgacaagggaagcggcggcaucaagggcgguuucgugcaccagcggaugaagagcaagaucggccggugguacagccggaccaugagcaagaccgagcggaugggcaugggccuguacguaaaguacggaggggaucccugggcugacagcgacgcccugaccuucagcggcgugauggugagcaugaaggagcccggcugguacagcuucggcuucgagaucaaggacaagaagugcgacgugcccugcaucggcaucgagauggugcacgacggcggcaaggagaccuggcacucugccgccacugccaucuacugccugaugggcagcggccagcugcugugggacaccgugaccggcguggacauggcccug

SEQ ID NO: 62 - protein, 469 aa, Artificial sequence
  Synthetic
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKGRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 63 - protein, 469 aa, Artificial sequence
  Synthetic
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQDSECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 64 - protein, 466 aa, Artificial sequence
  Synthetic
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREGRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 65 - protein, 466 aa, Artificial sequence
  Synthetic
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQDSACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 66 - protein, 466 aa, Artificial sequence
  Synthetic
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGGRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALTFSGVMVSMKEPGWYSFGFEIKDKKCDVPCIGIEMVHDGGKETWHSAATAIYCLMGSGQLLWDTVTGVDMAL

SEQ ID NO: 67 - protein, 466 aa, Artificial sequence
  Synthetic
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQDSACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALTFSGVMVSMKEPGWYSFGFEIKDKKCDVPCIGIEMVHDGGKETWHSAATAIYCLMGSGQLLWDTVTGVDMAL

SEQ ID NO: 68 - RNA, 9 nt, Artificial sequence
  Synthetic
ccrccaugg